SEQUENCE LISTING (2 sequences)

SEQ ID NO: 1 - protein, 21 aa, Artificial Sequence
  Modified Insulin A-chain
XIVEQCCTSCCSLEQLENYCN

SEQ ID NO: 2 - protein, 29 aa, Artificial Sequence
  Modified Insulin B-chain
FVCQHLCGSHLVEALYLVCGERGFHYTPX

(19) United States
(12) Patent Application Publication
(10) Pub. No.: US 2014/0357838 A1
(43) Pub. Date: Dec. 4, 2014

(54) N-Terminally Modified Insulin Derivatives

(71) Applicant: Novo Nordisk A/S, Bagsværd (DK)

(72) Inventors: Peter Madsen, Bagsvaerd (DK); Thomas B. Kjeldsen, Virum (DK); Tina M. Tagmose, Ballerup (DK)

(73) Assignee: NOVO NORDISK A/S, Bagsvaerd (DK)

(21) Appl. No.: 14/366,987

(22) PCT Filed: Dec. 21, 2012
(86) PCT No.: PCT/EP2012/076650

§ 371 Date: Jun. 19, 2014

Related U.S. Application Data
(60) Provisional application No. 61/593,636, filed on Feb. 1, 2012.

(30) Foreign Application Priority Data
Dec. 21, 2011 (EP) 11194898.0

(51) Int. Cl. C07K 14/62

(52) U.S. Cl. CPC C07K 14/62
USPC 530/303

(57) ABSTRACT

The invention is related to novel N-terminally modified insulin derivatives comprising extra disulphide bond(s), pharmaceutical compositions comprising such and methods of making such.

### FIELD OF THE INVENTION

The present invention is related to novel N-terminally modified insulins comprising one or more additional disulfide bonds, and methods of making such.

### BACKGROUND OF THE INVENTION

Diabetes mellitus is a metabolic disorder in which the ability to utilize glucose is partly or completely lost. The disorder may e.g. be treated by administering insulin.

The oral route is by far the most widely used route for drug administration. Administration of insulin is however often limited to parenteral routes rather than the preferred oral administration due to several barriers such as enzymatic degradation in the gastrointestinal (GI) tract and intestinal mucosa, drug efflux pumps, insufficient and variable absorption from the intestinal mucosa, as well as first pass metabolism in the liver.

Human insulin consists of two polypeptide chains, the A and B chains which contain 21 and 30 amino acid residues, respectively. The A and B chains are interconnected by two disulfide bridges. Human insulin is rapidly degraded in the lumen of gastrointestinal tract by the action of multiple proteases limiting its absorption into circulation. Insulin analogues that are hydrophilic and stabilized towards proteolytic degradation show higher bioavailability in animal models when compared to native insulin.

Incorporation of disulfide bonds into proteins is one of nature's ways of improving protein stability. There are also many examples of disulfide bonds being successfully engineered into proteins with concomitant increases in stability. To date, there are no reports of engineered disulfide bonds in insulin.

WO 2008/145721 is related to certain peptides which have been N-terminally modified to protect said peptides against degradation by aminopeptidases and dipeptidyl peptidases. WO 2010/033220 describes peptide conjugates coupled to polymers and optionally one or more moieties with up to ten carbon atoms.

Pharmaceutical compositions of therapeutic peptides are required to have a shelf life of several years in order to be suitable for common use. However, peptide compositions are inherently unstable due to sensitivity towards chemical and physical degradation.

WO 08/145728, WO 2010/060667 and WO 2011/086093 disclose examples of lipid pharmaceutical compositions for oral administration.

Pharmaceutical compositions often contain aldehyde and ketones in concentrations up to 200 ppm. Aldehyde and ketones may react with insulin and thus give rise to extensive chemical degradation of the insulin in the composition. As a result, the shelf life of the insulin composition may be below 3 months. Pharmaceutical drug development requires at least 2 years of shelf life.

It is known that aqueous pharmaceutical compositions can comprise compounds such as ethylenediamine for stability purposes. For example WO2006/125763 describes aqueous pharmaceutical polypeptide compositions comprising ethylenediamine as a buffer.

However, a method remains to be found for stabilising insulin in pharmaceutical compositions, especially non-aqueous lipid compositions, without adding ethylene diamine or other stabilizing compounds to the composition.

### SUMMARY OF THE INVENTION

The present invention is related to N-terminally modified insulins consisting of a peptide part, N-terminal modification groups and an albumin binding moiety, wherein the peptide part has at least one disulphide bond which is not present in human insulin.

In one aspect, the invention is related to N-terminally modified insulins consisting of a peptide part, N-terminal modification groups and an albumin binding moiety, wherein the peptide part has two or more cysteine substitutions and the three disulfide bonds of human insulin are retained.

In one aspect, the invention is related to N-terminally modified insulins comprising two or more cysteine substitutions, wherein the three disulfide bonds of human insulin are retained, wherein the sites of cysteine substitutions are chosen in such a way that the introduced cysteine residues are placed in the three dimensional structure of the folded N-terminally modified insulin to allow for the formation of one or more additional disulfide bonds not present in human insulin, and wherein the N-terminally modified insulin has at least 5% of the insulin receptor affinity of an insulin peptide having the same peptide part, the same N-terminal modification groups and the same albumin binding moiety but without any disulphide bonds which are not present in human insulin. In one aspect, the N-terminally modified insulin has at least 5% of the insulin receptor affinity of an insulin peptide having the same peptide part, the disulphide bonds which are present in human insulin and the same albumin binding moiety but without N-terminal modification groups.

In one aspect, N-terminally modified insulins according to the invention comprise modification groups which are one or two organic substituents which are each having a molecular weight (MW) below 200 g per mol and which are conjugated to the N-terminals of the parent insulin.

The present invention also relates to pharmaceutical compositions comprising an N-terminally modified insulin according to the invention.

### DESCRIPTION OF THE INVENTION

The present invention is related to novel N-terminally modified insulins, also herein named N-terminally protected insulins, wherein one or more disulfide bonds are engineered into the insulins. The novel N-terminally modified insulins according to the invention are particularly suitable for use in oral formulations. An aspect of the invention thus also contemplates oral pharmaceutical compositions comprising novel N-terminally modified insulins, wherein one or more disulfide bonds are engineered into the insulins.

In one aspect, the N-terminally modified insulins of the invention consist of a peptide part, an N-terminal modification group and an albumin binding moiety.

In one aspect an N-terminally modified insulin according to the invention has two or more cysteine substitutions and the three disulfide bonds of human insulin are retained.

In one aspect an N-terminally modified insulin of the invention has a side-chain. In one aspect the side-chain is attached to the epsilon amino group of a lysine residue. In one aspect the side-chain is attached to the epsilon amino group of a lysine residue in the B-chain.

In one aspect an N-terminally modified insulin according to the invention has two or more cysteine substitutions, the three disulfide bonds of human insulin retained and a side-chain which is attached to the epsilon amino group of a lysine residue such as in the B-chain.

In one aspect of the invention, the sites of cysteine substitutions are chosen in such a way that the introduced cysteine residues are placed in the three dimensional structure of the folded N-terminally modified insulin to allow for the formation of one or more additional disulfide bonds not present in human insulin.

It has surprisingly been found by the inventors that the N-terminally modified insulins according to the invention are stable in pharmaceutical compositions comprising aldehydes and/or ketones, such as trace amounts thereof, while the biological and pharmacological properties of the insulins are retained when compared to parent insulins, i.e. the similar insulins without N-terminal modification.

It has surprisingly been found by the inventors that the N-terminally modified insulins according to the invention are physically stable and are not prone towards fibrillation, while the biological and pharmacological properties of the insulins are retained when compared to parent insulins, i.e. the similar insulins without N-terminal modification.

In one aspect of the invention, N-terminally modified insulins according to the invention are used in aqueous formulations for subcutaneous injection insulin therapy.

In one aspect of the invention, N-terminally modified insulins according to the invention are useful as ultra-long acting insulins either as injection therapy in aqueous formulations or as oral therapy.

In one aspect the N-terminal modification of the N-terminally modified insulins according to the invention, in addition to conferring chemical stability towards aldehydes and/or ketones, may alter the insulin receptor affinity. For example, as described below, N-terminal modifications which at physiological pH render the N-terminals either neutral or negatively charged may confer a lower affinity for the insulin receptor.

A further aspect of this invention relates to furnishing of N-terminally modified insulins according to the invention, such as acylated N-terminally modified insulins according to the invention, which, when administered orally, have satisfactory bioavailabilities. Compared with the bioavailabilities of similar insulins without the N-terminal modification (parent insulins) given in similar doses, the bioavailability of preferred N-terminally modified insulins of this invention is similar. In one aspect the bioavailability is at least 10% higher than the bioavailability of similar acylated insulins with additional disulfide bridge(s) but without the N-terminal modification given in similar doses, in one aspect the bioavailability is at least 20% higher, in one aspect the bioavailability is at least 25% higher, in one aspect the bioavailability is at least 30% higher, in one aspect the bioavailability is at least 35% higher, in one aspect the bioavailability is at least 40% higher, in one aspect the bioavailability is at least 45% higher, in one aspect the bioavailability is at least 50% higher, in one aspect the bioavailability is at least 55% higher, in one aspect the bioavailability is at least 60% higher, in one aspect the bioavailability is at least 65% higher, in one aspect the bioavailability is at least 70% higher, in one aspect the bioavailability is at least 80% higher, in one aspect the bioavailability is at least 90% higher, in one aspect the bioavailability is at least 100% higher, in one aspect the bioavailability is more than 100% higher than that of the parent insulins.

When used herein the term “parent insulin” shall mean a similar insulin without the N-terminal modification. For example if the N-terminally modified insulin is an acylated N-terminally modified insulin, then the parent insulin is an acylated insulin with the same peptide part and the same albumin binding moiety but without the N-terminal modification, or for example if the N-terminally modified insulin is an acylated, protease stabilised N-terminally modified insulin, then the parent insulin is an acylated, protease stabilised insulin with the same peptide part and the same albumin binding moiety but without N-terminal modification.

In one aspect the bioavailability is at least 10% higher than the bioavailability of similar acylated and N-terminally modified insulins without any disulphide bonds which are not present in human insulin given in similar doses, in one aspect the bioavailability is at least 20% higher, in one aspect the bioavailability is at least 25% higher, in one aspect the bioavailability is at least 30% higher, in one aspect the bioavailability is at least 35% higher, in one aspect the bioavailability is at least 40% higher, in one aspect the bioavailability is at least 45% higher, in one aspect the bioavailability is at least 50% higher, in one aspect the bioavailability is at least 55% higher, in one aspect the bioavailability is at least 60% higher, in one aspect the bioavailability is at least 65% higher, in one aspect the bioavailability is at least 70% higher, in one aspect the bioavailability is at least 80% higher, in one aspect the bioavailability is at least 90% higher, in one aspect the bioavailability is at least 100% higher, in one aspect the bioavailability is more than 100% higher than that of the similar acylated and N-terminally modified insulin without any disulphide bonds which are not present in human insulin.

A further aspect of this invention relates to furnishing of N-terminally modified insulins according to the invention which, when administered orally, have satisfactory bioavailabilities relative to when administered as i.v. administration. Bioavailabilities of preferred compounds of this invention (relative to i.v. administration) are at least 0.3%, in one aspect at least 0.5%, in one aspect at least 1%, in one aspect at least 1.5%, in one aspect at least 2%, in one aspect at least 2.5%, in one aspect at least 3%, in one aspect at least 3.5%, in one aspect at least 4%, in one aspect at least 5%, in one aspect at least 6%, in one aspect at least 7%, in one aspect at least 8%, in one aspect at least 9%, in one aspect at least 10% relative to the bioavailability when the N-terminally modified insulin is administered i.v.

A further aspect of this invention relates to furnishing of N-terminally modified insulins according to the invention which, when administered orally, have satisfactory bioavailabilities relative to when administered as s.c. (subcutaneous) administration. Bioavailabilities of preferred compounds of this invention (relative to s.c. administration) are at least 0.3%, in one aspect at least 0.5%, in one aspect at least 1%, in one aspect at least 1.5%, in one aspect at least 2%, in one aspect at least 2.5%, in one aspect at least 3%, in one aspect at least 3.5%, in one aspect at least 4%, in one aspect at least 5%, in one aspect at least 6%, in one aspect at least 7%, in one aspect at least 8%, in one aspect at least 9%, in one aspect at least 10% relative to the bioavailability when the N-terminally modified insulin is administered s.c.

A further aspect of this invention relates to furnishing of N-terminally modified insulins according to the invention which, when administered subcutaneously, have satisfactory bioavailabilities relative to when administered as i.v. administration. Bioavailabilities of preferred compounds of this invention (relative to i.v. administration) are at least 10%, in one aspect at least 15%, in one aspect at least 20%, in one aspect at least 25%, in one aspect at least 30%, in one aspect at least 35%, in one aspect at least 40%, in one aspect at least 45%, in one aspect at least 50%, in one aspect at least 55%, in one aspect at least 60%, in one aspect at least 70%, in one aspect at least 80%, in one aspect at least 90%, relative to the bioavailability when the N-terminally modified insulin is administered i.v.

Standard assays for measuring insulin bioavailability are known to the person skilled in the art and include inter alia measurement of the relative areas under the curve (AUC) for the concentration of the insulin in question administered orally and intra venously (i.v.) in the same species. Quantitation of insulin concentrations in blood (plasma) samples can be done using for example antibody assays (ELISA) or by mass spectrometry.

A further aspect of this invention relates to furnishing of N-terminally modified insulins according to the invention which have satisfactory potencies. Compared with the potency of human insulin, potencies of preferred N-terminally modified insulins of the invention may be at least 5%, in one aspect at least 10%, in one aspect at least 20%, in one aspect at least 30%, in one aspect at least 40%, in one aspect at least 50%, in one aspect at least 75% and in one aspect at least 100% of the potency of human insulin.

Apparent in vivo potency can be measured by comparison of blood glucose versus time profiles of the insulin in question with the comparator insulin given in similar doses. Other means to measure in vivo potency are given in the examples.

Standard assays for measuring insulin in vitro potency are known to the person skilled in the art and include inter alia (1) insulin radioreceptor assays, in which the relative potency of an insulin is defined as the ratio of insulin to insulin analogue derivative required to displace 50% of ¹²⁵I-insulin specifically bound to insulin receptors e.g. present on cell membranes, e.g., a rat liver plasma membrane fraction; (2) lipogenesis assays, performed, e.g., with rat adipocytes, in which relative insulin potency is defined as the ratio of insulin to insulin analogue/derivative required to achieve 50% of the maximum conversion of [3-³H] glucose into organic-extractable material (i.e. lipids); (3) glucose oxidation assays in isolated fat cells in which the relative potency of the insulin analogue/derivative is defined as the ratio of insulin to insulin analogue/derivative to achieve 50% of the maximum conversion of glucose-1-[¹⁴C] into [¹⁴CO₂]; (4) insulin radioimmunoassays which can determine the immunogenicity of insulin analogues/derivatives by measuring the effectiveness by which insulin or an insulin analogue/derivative competes with ¹²⁵I-insulin in binding to specific anti-insulin antibodies; and (5) other assays which measure the binding of insulin or an insulin analogue or derivative to antibodies in animal blood plasma samples, such as ELISA assays possessing specific insulin antibodies.

N-terminally modified insulins according to the invention may have a prolonged time-action profile, i.e. provide an insulin effect in hyperglycemic, e.g., diabetic, patients that lasts longer than human insulin. In other words, an insulin with a prolonged time-action profile has prolonged lowering of the glucose level compared to human insulin. In one aspect, the N-terminally modified insulin according to the invention provides an insulin effect for from about 8 hours to about 2 weeks after a single administration of the insulin molecule. In one aspect, the insulin effect lasts from about 24 hours to about 2 weeks. In one aspect, the effect lasts from about 24 hours to about 1 week. In a further aspect, the effect lasts from about 1 week to about 2 weeks. In yet a further aspect, the effect lasts about 1 week. In yet a further aspect, the effect lasts about 2 weeks. In one aspect, the effect lasts from about 1 day to about 7 days. In one aspect, the effect lasts from about 1 day to about 3 days. In one aspect, the effect lasts from about 1 day to about 2 days. In one aspect, the effect lasts from about 2 days to about 3 days. In a further aspect, the effect lasts from about 7 days to about 14 days.

In yet a further aspect, the effect lasts about 7 days. In yet a further aspect, the effect lasts about 14 days. In one aspect, the effect lasts from about 2 days to about 7 days. In one aspect, the effect lasts from about 3 days to about 7 days. In yet a further aspect, the effect lasts about 3 days. In yet a further aspect, the effect lasts about 4 days. In yet a further aspect, the effect lasts about 5 days. In yet a further aspect, the effect lasts about 6 days. In yet a further aspect, the effect lasts about 7 days.

In one aspect, the N-terminally modified insulin according to the invention provides an insulin effect for from about 8 hours to about 24 hours after a single administration of the insulin molecule. In one aspect, the insulin effect lasts from about 10 hours to about 24 hours. In one aspect, the effect lasts from about 12 hours to about 24 hours. In a further aspect, the effect lasts from about 16 hours to about 24 hours. In yet a further aspect, the effect lasts from about 20 hours to about 24 hours. In yet a further aspect, the effect lasts about 24 hours.

In one aspect, the insulin effect lasts from about 24 hours to about 96 hours. In one aspect, the insulin effect lasts from about 24 hours to about 48 hours. In one aspect, the insulin effect lasts from about 24 hours to about 36 hours. In one aspect, the insulin effect lasts from about 1 hour to about 96 hours. In one aspect, the insulin effect lasts from about 1 hour to about 48 hours. In one aspect, the insulin effect lasts from about 1 hour to about 36 hours.

Duration of action (time-action profile) can be measured by the time that blood glucose is suppressed, or by measuring relevant pharmacokinetic properties, for example t_{1/2} or MRT (mean residence time).

A further aspect of this invention relates to the furnishing of N-terminally modified insulins according to the invention having a satisfactory prolonged action following oral administration relative to human insulin. Compared with human insulin, the duration of action of preferred N-terminally modified insulins of this invention is at least 10% longer. In one aspect the duration is at least 20% longer, in one aspect at least 25% longer, in one aspect at least 30% longer, in one aspect at least 35% longer, in one aspect at least 40% longer, in one aspect at least 45% longer, in one aspect at least 50% longer, in one aspect at least 55% longer, in one aspect at least 60% longer, in one aspect at least 65% longer, in one aspect at least 70% longer, in one aspect at least 80% longer, in one aspect at least 90% longer, in one aspect at least 100% longer, in one aspect more than 100% longer than that of human insulin.

In one aspect, compared with a once daily insulin such as LysB29(Nε-tetradecanoyl)desB30human insulin or A21Gly, B31Arg, B32Arg human insulin, the duration of action of preferred N-terminally modified insulins of this invention is at least 10% longer. In one aspect the duration is at least 20% longer, in one aspect at least 25% longer, in one aspect at least 30% longer, in one aspect at least 35% longer, in one aspect at least 40% longer, in one aspect at least 45% longer, in one aspect at least 50% longer, in one aspect at least 55% longer, in one aspect at least 60% longer, in one aspect at least 65% longer, in one aspect at least 70% longer, in one aspect at least 80% longer, in one aspect at least 90% longer, in one aspect at least 100% longer, in one aspect more than 100% longer than that of a once daily insulin such as LysB29(Nε-tetradecanoyl)desB30 human insulin or A21Gly, B31Arg, B32Arg human insulin.

In one aspect, compared with a once daily insulin such as LysB29(Nε-tetradecanoyl)desB30 human insulin or A21Gly, B31Arg, B32Arg human insulin, the duration of action of preferred N-terminally modified insulins of this invention is at least 100% longer. In one aspect the duration is at least 200% longer, in one aspect at least 250% longer, in one aspect at least 300% longer, in one aspect at least 350% longer, in one aspect at least 400% longer, in one aspect at least 450% longer, in one aspect at least 500% longer, in one aspect at least 550% longer, in one aspect at least 600% longer, in one aspect at least 650% longer, in one aspect at least 700% longer, in one aspect at least 800% longer, in one aspect at least 900% longer, in one aspect at least 1000% longer, in one aspect more than 1000% longer than that of a once daily insulin such as LysB29(Nε-tetradecanoyl)desB30 human insulin or A21Gly, B31Arg, B32Arg human insulin.

In one aspect the N-terminally modified insulins of the invention are stabilized against proteolytic degradation, i.e. against rapid degradation in the gastro intestinal (GI) tract or elsewhere in the body. In one aspect the N-terminally modified insulins of the invention are stabilized against proteolytic degradation relative to the N-terminally modified insulin without one or more additional disulfide bonds. In one aspect the N-terminally modified insulins of the invention are stabilized against proteolytic degradation relative to similar acylated insulins with additional disulfide bridge(s) but without the N-terminal modification given in similar doses.

An N-terminally modified insulin which is stabilized against proteolytic degradation is herein to be understood as an N-terminally modified insulin of the invention, which is subjected to slower degradation by one or more proteases relative to human insulin. In one embodiment an N-terminally modified insulin according to the invention is subjected to slower degradation by one or more proteases relative to human insulin. In a further embodiment of the invention an N-terminally modified insulin according to the invention is stabilized against degradation by one or more enzymes selected from the group consisting of: pepsin (such as e.g. the isoforms pepsin A, pepsin B, pepsin C and/or pepsin F), chymotrypsin (such as e.g. the isoforms chymotrypsin A, chymotrypsin B and/or chymotrypsin C), trypsin, Insulin-Degrading Enzyme (IDE), elastase (such as e.g. the isoforms pancreatic elastase I and/or II), carboxypeptidase (e.g. the isoforms carboxypeptidase A, carboxypeptidase A2 and/or carboxypeptidase B), aminopeptidase, cathepsin D and other enzymes present in intestinal extracts derived from rat, pig or human.

In one embodiment an N-terminally modified insulin according to the invention is stabilized against degradation by one or more enzymes selected from the group consisting of: chymotrypsin, trypsin, Insulin-Degrading Enzyme (IDE), elastase, carboxypeptidases, aminopeptidases and cathepsin D. In a further embodiment an N-terminally modified insulin according to the invention is stabilized against degradation by one or more enzymes selected from the group consisting of: chymotrypsin, carboxypeptidases and IDE. In a yet further embodiment an N-terminally modified insulin according to the invention is stabilized against degradation by one or more enzymes selected from: chymotrypsin and IDE. In a yet further embodiment an N-terminally modified insulin according to the invention is stabilized against degradation by one or more enzymes selected from: chymotrypsin and carboxypeptidases.

A “protease” or a “protease enzyme” is a digestive enzyme which degrades proteins and peptides and which is found in various tissues of the human body such as e.g. the stomach (pepsin), the intestinal lumen (chymotrypsin, trypsin, elastase, carboxypeptidases, etc.) or mucosal surfaces of the GI tract (aminopeptidases, carboxypeptidases, enteropeptidases, dipeptidyl peptidases, endopeptidases, etc.), the liver (Insulin degrading enzyme, cathepsin D etc), and in other tissues.

T½ may be determined as a measure of the proteolytical stability of an N-terminally modified insulin according to the invention towards protease enzymes such as chymotrypsin, pepsin and/or carboxypeptidase A or towards a mixture of enzymes such as tissue extracts (from liver, kidney, duodenum, jejunum, ileum, colon, stomach, etc.). In one embodiment of the invention T½ is increased relative to human insulin. In a further embodiment T½ is increased relative to the N-terminally modified insulin without one or more additional disulfide bonds. In a yet further embodiment T½ is increased at least 2-fold relative to human insulin. In a yet further embodiment T½ is increased at least 2-fold relative to the N-terminally modified insulin without one or more additional disulfide bonds. In a yet further embodiment T½ is increased at least 3-fold relative to human insulin. In a yet further embodiment T½ is increased at least 3-fold relative to the N-terminally modified insulin without one or more additional disulfide bonds. In a yet further embodiment T½ is increased at least 4-fold relative to human insulin. In a yet further embodiment T½ is increased at least 4-fold relative to the N-terminally modified insulin without one or more additional disulfide bonds. In a yet further embodiment T½ is increased at least 5-fold relative to human insulin. In a yet further embodiment T½ is increased at least 5-fold relative to the N-terminally modified insulin without one or more additional disulfide bonds. In a yet further embodiment T½ is increased at least 10-fold relative to human insulin. In a yet further embodiment T½ is increased at least 10-fold relative to the N-terminally modified insulin without one or more additional disulfide bonds.

The term “stability” is herein used for a pharmaceutical composition comprising an N-terminally modified insulin to describe the shelf life of the composition. The term “stabilized” or “stable” when referring to an N-terminally modified insulin of the invention thus refers to a composition with increased chemical stability, increased physical stability or increased physical and chemical stability relative to a composition comprising an insulin which is not N-terminally modified or relative to an insulin without one or more additional disulfide bonds.

In one aspect, an N-terminally modified insulin according to the invention has improved chemical stability. In one aspect, an N-terminally modified insulin according to the invention has improved physical stability. In one aspect, an N-terminally modified insulin according to the invention has improved chemical and physical stability.

In one aspect, an N-terminally modified insulin according to the invention has improved chemical and/or physical stability relative to human insulin. In one aspect, an N-terminally modified insulin according to the invention has improved chemical and/or physical stability relative to the N-terminally modified insulin without one or more additional disulfide bonds. In one aspect, an N-terminally modified insulin according to the invention has improved chemical and/or physical stability relative to a similar acylated insulin with additional disulfide bridge(s) but without the N-terminal modification.

The term “physical stability” as used herein refers to the tendency of the N-terminally modified insulin to form biologically inactive and/or insoluble aggregates of the insulin as a result of exposure of the insulin to thermo-mechanical stresses and/or interaction with interfaces and surfaces that are destabilizing, such as hydrophobic surfaces and interfaces. Physical instability thus involves conformational changes relative to human insulin, which includes loss of higher order structure, aggregation, fibrillation, precipitation and/or adsorption to surfaces. Peptides such as insulin are known to be prone to instability due to e.g. fibrillation. Physical stability of a solution comprising the N-terminally modified insulin may be evaluated by conventional means of e.g. visual inspection, nephelometry and/or turbidity measurements after exposing the solution filled in suitable containers (e.g. cartridges or vials) to mechanical/physical stress (e.g. agitation) at different temperatures for various time periods. Visual inspection of the solution is performed in a sharp focused light with a dark background. The turbidity of the solution is characterized by a visual score ranking the degree of turbidity for instance on a scale from 0 to 3 (a solution showing no turbidity corresponds to a visual score 0, and a solution showing visual turbidity in daylight corresponds to visual score 3). A solution is classified physical unstable with respect to protein aggregation, when it shows visual turbidity in daylight. Alternatively, the turbidity of the solution can be evaluated by simple turbidity measurements well-known to the skilled person. Physical stability of the N-terminally modified insulins of the invention can also be evaluated by using a spectroscopic agent or probe of the conformational status of the N-terminally modified insulin. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths. Physical stability of the N-terminally modified insulins of the invention may e.g. be determined as described in example 109.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of an N-terminally modified insulin as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the pharmaceutical composition as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation can be mentioned as another variant of chemical degradation. The chemical stability of the N-terminally modified insulin can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size, hydrophilicity, hydrophobicity, and/or charge using various chromatography techniques (e.g. SEC-HPLC and/or RP-HPLC).

In one embodiment, an N-terminally modified insulin of the invention has little or no tendency to aggregate. The aggregation tendency is preferably significantly improved relatively to the aggregation tendency of human insulin and/or an N-terminally modified insulin without one or more additional disulfide bonds when tested in a thioflavin assay.

In one aspect, an N-terminally modified insulin according to the invention has improved thermodynamic stability such as e.g. folding stability, conformational stability and/or higher melting temperature.

When used herein an N-terminally modified insulin is said to have improved “thermodynamic stability” if denaturation of said derivative requires higher stress level such as higher temperature and/or higher concentration of denaturation agent in comparison to human insulin or an N-terminally modified insulin without one or more additional disulfide bonds.

Conformational stability may be evaluated by circular dichroism and NMR as e.g. described by Hudson and Andersen, Peptide Science, vol 76 (4), pp. 298-308 (2004). Melting temperature is understood as the temperature at which an insulin structure is reversibly or irreversibly changed. Higher melting temperature corresponds to more stable structures. Melting temperature can be determined e.g. by evaluating conformational stability by circular dichroism and/or NMR as a function of temperature or by differential scanning calorimetry. Thermodynamic stability can also be determined by CD spectroscopy and or NMR in the presence of increasing concentration of denaturation agent, such as for example guanidinium hydrochloride. Free energy of unfolding as described previously (Kaarsholm, N. C., et al, 1993, Biochemistry, 32, 10773-8) can be determined from such experiments. Upon protein denaturation, negative CD in the far UV range (240-218-nm) gradually diminishes, consistent with the loss of ordered secondary structure that accompanies protein unfolding (Holladay et al., 1977, Biochim. Biophys. Acta, 494, 245-254; Melberg and Johnson, 1990, Biochim. Biophys. Acta, 494, 245-254). The insulin CD spectrum in the near UV range (330-250-nm) reflects the environment of the tyrosine chromophore with contributions from the disulfide bonds (Morris et al., 1968, Biochim. Biophys. Acta., 160, 145-155; Wood et al., 1975, Biochim. Biophys. Acta, 160, 145-155; Strickland & Mercola, 1976, Biochemistry, 15, 3875-3884). The free energy of unfolding of insulin was previously calculated from such studies to be 4.5 kcal/mol (Kaarsholm, N. C., et al, 1993, Biochemistry, 32, 10773-8).

Insulin CD spectrum in the near UV range (330-250-nm) reflects the environment of the tyrosine chromophore with contributions from the disulfide bonds. Since tyrosine residues are part of the insulin's dimer surface, changes in molar ellipticity at this region (especially at 276 nm) reflect on insulin's association state. Another way to measure insulin's association state is by application of size-exclusion chromatography under non-dissociating conditions as known in the art and described in the examples.

The charge of the N-terminal modification group of the N-terminally modified insulin may be chosen so that the N-terminally modified insulin has retained or altered affinity for the insulin receptor (IR) compared to the insulin receptor affinity of the parent insulin.

For example, an N-terminal modification group which at physiological pH (i.e. pH 7.4) is neutral or negatively charged may result in reduced IR affinity compared to the parent insulin without N-terminal modification. As another example, an N-terminal modification group which at physiological pH is positively charged may result in retained or only slightly reduced IR affinity compared to the parent insulin without N-terminal modification.

According to an aspect of the invention, N-terminal modification groups for use in the invention may be neutral or positively charged or negatively charged at physiological pH.

In one aspect, the N-terminally modified insulin of the invention consists of a peptide part, an N-terminal modification group and an albumin binding moiety.

By the term “positively charged at physiological pH” when used about the N-terminal modification group as herein described is meant, that in a solution comprising the N-terminally modified polypeptide at least 10% of the N-terminal modification groups have a charge of +1 at physiological pH. In one aspect at least 30% of the N-terminal modification groups in a solution of the N-terminally modified polypeptide have a charge of +1 at physiological pH. In a further aspect at least 50% of the N-terminal modification groups in a solution of the N-terminally modified polypeptide have a charge of +1 at physiological pH. In yet a further aspect at least 70% of the N-terminal modification groups in a solution of the N-terminally modified polypeptide have a charge of +1 at physiological pH. In still a further aspect at least 90% of the N-terminal modification groups in a solution of the N-terminally modified polypeptide have a charge of +1 at physiological pH.

Examples of positively charged N-terminal modification groups at physiological pH include but is not limited to: N,N-di-C1-4alkyl such as N,N-dimethyl and N,N-diethyl, N-amidinyl, 4-(N,N-dimethylamino)butanoyl, 3-(1-piperidinyl)propionyl, 3-(N,N-dimethylamino)propionyl, and

### N,N-dimethyl-glycyl:

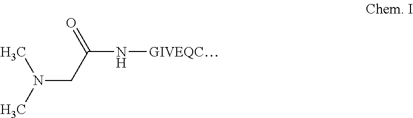

When used herein the term “neutral at physiological pH” when used about the N-terminal modification group as herein described is meant, that in a solution comprising the N-terminally modified insulin at least 10% of the N-terminal modification groups have a neutral charge (i.e. the charge is 0) at physiological pH. In one aspect at least 30% of the N-terminal modification groups in a solution of the N-terminally modified polypeptide have a neutral charge at physiological pH. In a further aspect at least 50% of the N-terminal modification groups in a solution of the N-terminally modified polypeptide have a neutral charge at physiological pH. In yet a further aspect at least 70% of the N-terminal modification groups in a solution of the N-terminally modified polypeptide have a neutral charge at physiological pH. In still a further aspect at least 90% of the N-terminal modification groups in a solution of the N-terminally modified polypeptide have a neutral charge at physiological pH.

Examples of neutral N-terminal modification groups at physiological pH include but is not limited to: Carbamoyl, thiocarbamoyl, and C1-4 chain acyl groups, such as formyl, acetyl, propionyl, butyryl, and

pyroglutamyl:

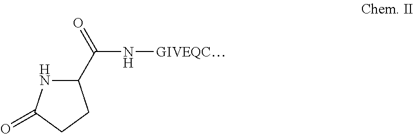

When used herein the term “negatively charged at physiological pH” when used about the N-terminal modification group as herein described is meant, that in a solution comprising the N-terminally modified insulin at least 10% of the N-terminal modification groups have a charge of −1 (i.e. minus 1) at physiological pH. In one aspect at least 30% of the N-terminal modification groups in a solution of the N-terminally modified polypeptide have a charge of −1 at physiological pH. In a further aspect at least 50% of the N-terminal modification groups in a solution of the N-terminally modified polypeptide have a charge of −1 at physiological pH. In yet a further aspect at least 70% of the N-terminal modification groups in a solution of the N-terminally modified polypeptide have a charge of −1 at physiological pH. In still a further aspect at least 90% of the N-terminal modification groups in a solution of the N-terminally modified polypeptide have a charge of −1 at physiological pH.

Examples of negatively charged N-terminal modification groups at physiological pH include but is not limited to: oxalyl, glutaryl, diglycolyl (other names: 3-oxoglutaryl and carboxymethoxyacetyl).

In one aspect, a negatively charged N-terminal modification group at physiological pH according to the invention is not malonyl or succinyl. In one aspect, a negatively charged N-terminal modification group at physiological pH according to the invention is not malonyl. In one aspect, a negatively charged N-terminal modification group at physiological pH according to the invention is not succinyl.

In one aspect of the invention, an N-terminally modified insulin is obtained, wherein the N-terminal modification is with one or more positively charged N-terminal modification groups.

In one aspect of the invention, an N-terminally modified insulin is obtained, wherein the N-terminal modification is with one or more negatively charged N-terminal modification groups.

In one aspect of the invention, an N-terminally modified insulin is obtained, wherein the N-terminal modification is with one or more neutral N-terminal modification groups.

In one aspect an N-terminally modified insulin according to the invention is obtained, wherein the peptide part is substituted with an albumin binding moiety in a position other than one of the N-terminals of the insulin. In one aspect the albumin binding moiety consists of a fatty acid or a difatty acid attached to the insulin optionally via a linker. The linker may be any suitable portion inbetween the fatty acid or the fatty diacid and the point of attachment to the insulin, which portion may also be referred to as a linker moiety, spacer, or the like.

In one aspect, a linker is present and comprises one or more entities selected from the group consisting of: Gly, D-Ala, L-Ala, D-αGlu, L-αGlu, D-γGlu, L-γGlu, D-αAsp, L-αAsp, D-βAsp, L-βAsp, βAla, 4-aminobutyric acid, 5-aminovaleric acid, 6-aminohexanoic acid, D-Glu-α-amide, L-Glu-α-amide, D-Glu-γ-amide, L-Glu-γ-amide, D-Asp-α-amide, L-Asp-α-amide, D-Asp-β-amide, L-Asp-β-amide, or:

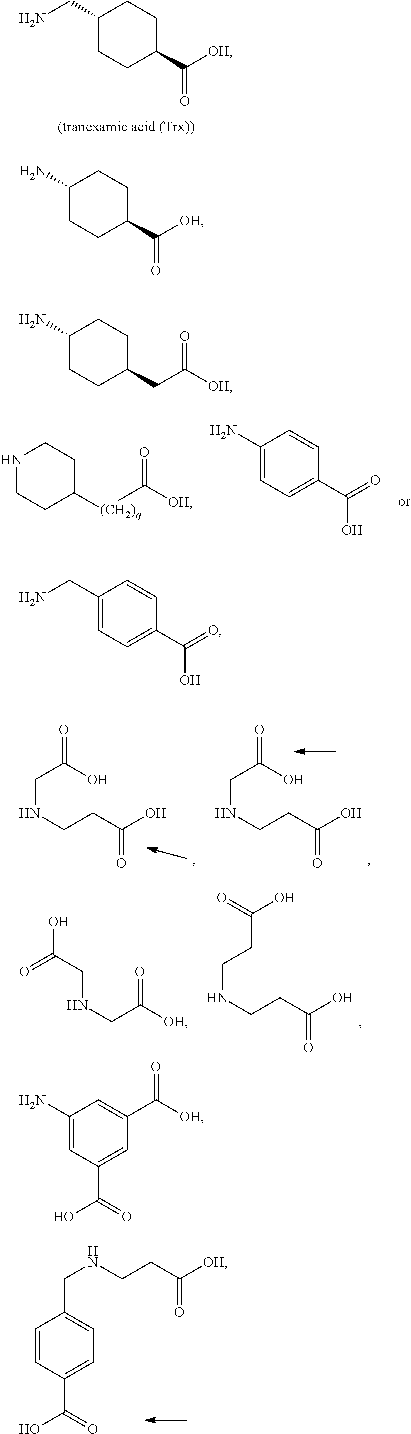

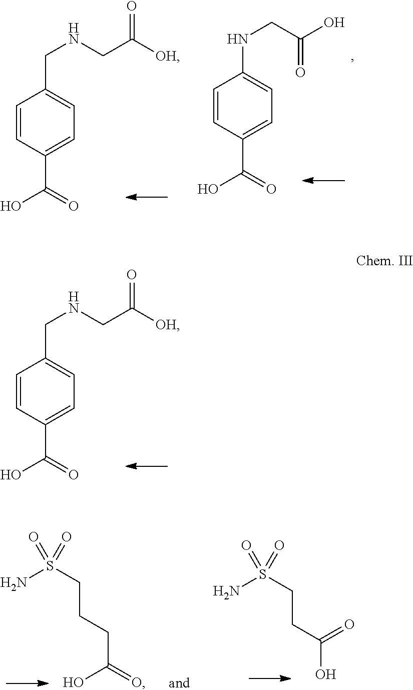

from which a hydrogen atom and/or a hydroxyl group has been removed, wherein q is 0, 1, 2, 3 or 4 and, in this embodiment may, alternatively, be 7-aminoheptanoic acid or 8-aminooctanoic acid and wherein the arrows indicate the attachment point to, or if more linkers are present, towards the amino group of the protease stabilised insulin.

In one aspect, a linker is present and comprises gamma-Glu (γGlu) entities, one or more OEG entities or a combination thereof.

Herein, the term “fatty acid” covers a linear or branched, aliphatic carboxylic acids having at least 14 carbon atoms and being saturated or unsaturated. Non limiting examples of fatty acids are myristic acid, palmitic acid, and stearic acid.

Herein, the term “fatty diacid” covers a linear or branched, aliphatic dicarboxylic acids having at least 14 carbon atoms and being saturated or unsaturated. Non limiting examples of fatty diacids are tetradecanedioic acid, hexadecanedioic acid, heptadecanedioic acid, octadecanedioic acid, and eicosanedioic acid.

Oral pharmaceutical compositions comprising N-terminally modified insulins as herein described are also contemplated by the invention. In one aspect an oral pharmaceutical composition is a composition comprising one or more lipids and an N-terminally modified insulin of the invention.

N-terminally modified insulins of the invention are surprisingly chemically stable when used in lipid pharmaceutical formulations. In one aspect, a lipid pharmaceutical formulation comprising an N-terminal modified insulin according to the invention is chemically stable for at least 2 weeks of usage and 1 year of storage. In one aspect, a lipid pharmaceutical formulation comprising an N-terminal modified insulin according to the invention is chemically stable for at least 4 weeks of usage and 1 year of storage. In one aspect, a lipid pharmaceutical formulation comprising an N-terminal modified insulin according to the invention is chemically stable for at least 4 weeks of usage and 2 years of storage. In one aspect, a lipid pharmaceutical formulation comprising an N-terminal modified insulin according to the invention is chemically stable for at least 6 weeks of usage and 2 years of storage.

It is known to the person skilled in the art that a common method for stabilizing insulins in aqueous pharmaceutical formulations is to add zinc to the pharmaceutical formulation and thereby form insulin hexamers with the zinc. In one aspect according to the invention, a pharmaceutical lipid composition comprising an N-terminally modified insulin of the invention and no zinc or only trace amounts of zinc is chemically stable similar to an aqueous pharmaceutical formulation comprising the N-terminal modified insulin and zinc.

It has surprisingly been found that non-aqueous liquid insulin pharmaceutical compositions comprising an N-terminally modified insulin, one or more lipids and optionally one or more surfactants are chemically stable. In one aspect the pharmaceutical composition of the invention comprises an N-terminally modified insulin, one or more lipids, one or more surfactants and a cosolvent. In one aspect of the invention the cosolvent is propylene glycol.

In one aspect of the invention, the N-terminally modified insulin is present in the pharmaceutical composition in a concentration between from 0.1 to 30% (w/w) of the total amount of ingredients in the composition. In another aspect the N-terminally modified insulin is present in a concentration between from 0.5 to 20% (w/w). In another aspect the N-terminally modified insulin is present in a concentration between from 1 to 10% (w/w).

In one aspect of the invention, the N-terminally modified insulin is present in the pharmaceutical composition in a concentration between from 0.2 mM to 100 mM. In another aspect the N-terminally modified insulin is present in a concentration between from 0.5 to 70 mM. In another aspect the N-terminally modified insulin is present in a concentration between from 0.5 to 35 mM. In another aspect the N-terminally modified insulin is present in a concentration between from 1 to 30 mM.

The term “lipid” is herein used for a substance, material or ingredient that is more mixable with oil than with water. A lipid is insoluble or almost insoluble in water but is easily soluble in oil or other nonpolar solvents.

A lipid, used for a pharmaceutical composition comprising an N-terminally modified insulin of the invention, may comprise one or more lipophilic substances, i.e. substances that form homogeneous mixtures with oils and not with water. Multiple lipids may constitute the lipophilic phase of the non-aqueous liquid pharmaceutical composition and form the oil aspect. At room temperature, the lipid can be solid, semisolid or liquid. For example, a solid lipid can exist as a paste, granular form, powder or flake. If more than one excipient comprises the lipid, the lipid can be a mixture of liquids, solids, or both.

Examples of solid lipids i.e., lipids which are solid or semisolid at room temperature, include, but are not limited to, the following:

1. Mixtures of mono-, di- and triglycerides, such as hydrogenated coco-glycerides (melting point (m.p.) of about 33.5° C. to about 37° C.], commercially-available as WITEPSOL HI5 from Sasol Germany (Witten, Germany); Examples of fatty acid triglycerides e.g., C10-C22 fatty acid triglycerides include natural and hydrogenated oils, such as vegetable oils;

2. Esters, such as propylene glycol (PG) stearate, commercially available as MONOSTEOL (m.p. of about 33° C. to about 36° C.) from Gattefosse Corp. (Paramus, N.J.); diethylene glycol palmito stearate, commercially available as HYDRINE (m.p. of about 44.5° C. to about 48.5° C.) from Gattefosse Corp.;

3. Polyglycosylated saturated glycerides, such as hydrogenated palm/palm kernel oil PEG-6 esters (m.p. of about 30.5° C. to about 38° C.), commercially-available as LABRAFIL M2130 CS from Gattefosse Corp. or Gelucire 33/01;

4. Fatty alcohols, such as myristyl alcohol (m.p. of about 39° C.), commercially available as LANETTE 14 from Cognis Corp. (Cincinnati, Ohio); esters of fatty acids with fatty alcohols, e.g., cetyl palmitate (m.p. of about 50° C.); isosorbid monolaurate, e.g. commercially available under the trade name ARLAMOL ISML from Uniqema (New Castle, Del.), e.g. having a melting point of about 43° C.;

5. PEG-fatty alcohol ether, including polyoxyethylene (2) cetyl ether, e.g. commercially available as BRIJ 52 from Uniqema, having a melting point of about 33° C., or polyoxyethylene (2) stearyl ether, e.g. commercially available as BRIJ 72 from Uniqema having a melting point of about 43° C.;

6. Sorbitan esters, e.g. sorbitan fatty acid esters, e.g. sorbitan monopalmitate or sorbitan monostearate, e.g, commercially available as SPAN 40 or SPAN 60 from Uniqema and having melting points of about 43° C. to 48° C. or about 53° C. to 57° C. and 41° C. to 54° C., respectively; and

7. Glyceryl mono-C6-C14-fatty acid esters. These are obtained by esterifying glycerol with vegetable oil followed by molecular distillation. Monoglycerides include, but are not limited to, both symmetric (i.e. β-monoglycerides) as well as asymmetric monoglycerides (α-monoglycerides). They also include both uniform glycerides (in which the fatty acid constituent is composed primarily of a single fatty acid) as well as mixed glycerides (i.e. in which the fatty acid constituent is composed of various fatty acids). The fatty acid constituent may include both saturated and unsaturated fatty acids having a chain length of from e.g. C8-C14. Particularly suitable are glyceryl mono laurate e.g. commercially available as IMWITOR 312 from Sasol North America (Houston, Tex.), (m.p. of about 56° C.-60° C.); glyceryl mono dicocoate, commercially available as IMWITOR 928 from Sasol (m.p. of about 33° C.-37° C.); monoglyceryl citrate, commercially available as IMWITOR 370, (m.p. of about 59 to about 63° C.); or glyceryl mono stearate, e.g., commercially available as IMWITOR 900 from Sasol (rn.p. of about 56° C.-61° C.); or self-emulsifying glycerol mono stearate, e.g., commercially available as IMWITOR 960 from Sasol (m.p. of about 56° C.-61° C.).

Examples of liquid and semisolid lipids, i.e., lipids which are liquid or semisolid at room temperature include, but are not limited to, the following:

1. Mixtures of mono-, di- and triglycerides, such as medium chain mono- and diglycerides, glyceryl caprylate/caprate, commercially-available as CAPMUL MCM from Abitec Corp. (Columbus, Ohio); and glycerol monocaprylate, commercially available as RYLO MG08 Pharma and glycerol monocaprate, commercially available as RYLO MG10 Pharma from DANISCO.

2. Glyceryl mono- or di fatty acid ester, e.g. of C6-C18, e.g. C6-C16 e.g. C8-C10, e.g. C8, fatty acids, or acetylated derivatives thereof, e.g. MYVACET 9-45 or 9-08 from Eastman Chemicals (Kingsport, Tenn.) or IMWITOR 308 or 312 from Sasol;

3. Propylene glycol mono- or di-fatty acid ester, e.g. of C8-C20, e.g. C8-C12, fatty acids, e.g. LAUROGLYCOL 90, SEFSOL 218, or CAPRYOL 90 or CAPMUL PG-8 (same as propylene glycol caprylate) from Abitec Corp. or Gattefosse;

4. Oils, such as safflower oil, sesame oil, almond oil, peanut oil, palm oil, wheat germ oil, corn oil, castor oil, coconut oil, cotton seed oil, soybean oil, olive oil and mineral oil;

5. Fatty acids or alcohols, e.g. C8-C20, saturated or mono- or di-unsaturated, e.g. oleic acid, oleyl alcohol, linoleic acid, capric acid, caprylic acid, caproic acid, tetradecanol, dodecanol, decanol;

6. Medium chain fatty acid triglycerides, e.g. C8-C12, e.g. MIGLYOL 812, or long chain fatty acid triglycerides, e.g. vegetable oils;

7. Transesterified ethoxylated vegetable oils, e.g. commercially available as LABRAFIL M2125 CS from Gattefosse Corp;

8. Esterified compounds of fatty acid and primary alcohol, e.g. C8-C20, fatty acids and C2-C3 alcohols, e.g. ethyl linoleate, e.g. commercially available as NIKKOL VF-E from Nikko Chemicals (Tokyo, Japan), ethyl butyrate, ethyl caprylate oleic acid, ethyl oleate, isopropyl myristate and ethyl caprylate;

9. Essential oils, or any of a class of volatile oils that give plants their characteristic odours, such as spearmint oil, clove oil, lemon oil and peppermint oil;

10. Fractions or constituents of essential oils, such as menthol, carvacrol and thymol;

11. Synthetic oils, such as triacetin, tributyrin;

12. Triethyl citrate, acetyl triethyl citrate, tributyl citrate, acetyl tributyl citrate;

13. Polyglycerol fatty acid esters, e.g. diglyceryl monooleate, e.g. DGMO-C, DGMO-90, DGDO from Nikko Chemicals; and

14. Sorbitan esters, e.g. sorbitan fatty acid esters, e.g. sorbitan monolaurate, e.g. commercially available as SPAN 20 from Uniqema.

15. Phospholipids, e.g. Alkyl-O-Phospholipids, Diacyl Phosphatidic Acids, Diacyl Phosphatidyl Cholines, Diacyl Phosphatidyl Ethanolamines, Diacyl Phosphatidyl Glycerols, Di-O-Alkyl Phosphatidic Acids, L-alpha-Lysophosphatidylcholines (LPC), L-alpha-Lysophosphatidylethanolamines (LPE), L-alpha-Lysophosphatidylglycerol (LPG), L-alpha-Lysophosphatidylinositols (LPI), L-alpha-Phosphatidic acids (PA), L-alpha-Phosphatidylcholines (PC), L-alpha-Phosphatidylethanolamines (PE), L-alpha-Phosphatidylglycerols (PG), Cardiolipin (CL), L-alpha-Phosphatidylinositols (PI), L-alpha-Phosphatidylserines (PS), Lyso-Phosphatidylcholines, Lyso-Phosphatidylglycerols, sn-Glycerophosphorylcholines commercially available from LARODAN, or soybean phospholipid (Lipoid S100) commercially available from Lipoid GmbH.

16. Polyglycerol fatty acid esters, such as polyglycerol oleate (Plurol Oleique from Gattefosse).

In one aspect of the invention, the lipid is one or more selected from the group consisting of mono-, di-, and triglycerides. In a further aspect, the lipid is one or more selected from the group consisting of mono- and diglycerides. In yet a further aspect, the lipid is Capmul MCM or Capmul PG-8. In a still further aspect, the lipid is Capmul PG-8. In a further aspect the lipid is Glycerol monocaprylate (Rylo MG08 Pharma from Danisco).

In one aspect the lipid, used for a pharmaceutical composition comprising an N-terminally modified insulin of the invention, is selected from the group consisting of: Glycerol mono-caprylate (such as e.g. Rylo MG08 Pharma) and Glycerol mono-caprate (such as e.g. Rylo MG10 Pharma from Danisco). In another aspect the lipid is selected from the group consisting of: propyleneglycol caprylate (such as e.g. Capmul PG8 from Abitec or Capryol PGMC, or Capryol 90 from Gattefosse).

In one aspect of the invention, the lipid is present in the pharmaceutical composition in a concentration between from 10% to 90% (w/w) of the total amount of ingredients including insulin in the composition. In another aspect the lipid is present in a concentration between from 10 to 80% (w/w). In another aspect the lipid is present in a concentration between from 10 to 60% (w/w). In another aspect the lipid is present in a concentration between from 15 to 50% (w/w). In another aspect the lipid is present in a concentration between from 15 to 40% (w/w). In another aspect the lipid is present in a concentration between from 20 to 30% (w/w). In another aspect the lipid is present in a concentration of about 25% (w/w).

In one aspect of the invention, the lipid is present in the pharmaceutical composition in a concentration between from 100 mg/g to 900 mg/g of the total amount of ingredients including insulin in the composition. In another aspect the lipid is present in a concentration between from 100 to 800 mg/g. In another aspect the lipid is present in a concentration between from 100 to 600 mg/g. In another aspect the lipid is present in a concentration between from 150 to 500 mg/g. In another aspect the lipid is present in a concentration between from 150 to 400 mg/g. In another aspect the lipid is present in a concentration between from 200 to 300 mg/g. In another aspect the lipid is present in a concentration of about 250 mg/g.

In one aspect of the invention, the cosolvent is present in the pharmaceutical composition in a concentration between from 0% to 30% (w/w) of the total amount of ingredients including insulin in the composition. In another aspect the cosolvent is present in a concentration between from 5% to 30% (w/w). In another aspect the cosolvent is present in a concentration between from 10 to 20% (w/w).

In one aspect of the invention, the cosolvent is present in the pharmaceutical composition in a concentration between from 0 mg/g to 300 mg/g of the total amount of ingredients including insulin in the composition. In another aspect the cosolvent is present in a concentration between from 50 mg/g to 300 mg/g. In another aspect the cosolvent is present in a concentration between from 100 to 200 mg/g.

In one aspect of the invention the oral pharmaceutical composition does not contain oil or any other lipid component or surfactant with an HLB below 7. In a further aspect the composition does not contain oil or any other lipid component or surfactant with an HLB below 8. In a yet further aspect the composition does not contain oil or any other lipid component or surfactant with an HLB below 9. In a yet further aspect the composition does not contain oil or any other lipid component or surfactant with an HLB below 10.

The hydrophilic-lipophilic balance (HLB) of each of the non-ionic surfactants of the liquid non-aqueous pharmaceutical composition of the invention is above 10 whereby high insulin peptide (such as N-terminally modified insulin) drug loading capacity and high oral bioavailability are achieved. In one aspect the non-ionic surfactants according to the invention are non-ionic surfactants with HLB above 11. In one aspect the non-ionic surfactants according to the invention are non-ionic surfactants with HLB above 12.

The term “about” as used herein means in reasonable vicinity of the stated numerical value, such as plus or minus 10%.

A non-limiting example of lipid pharmaceutical compositions, for use as pharmaceutical compositions comprising an N-terminally modified insulin of the invention, may e.g. be found in the patent applications WO 08/145728, WO 2010/060667 and WO 2011/086093.

The peptide part of an N-terminally modified insulin according to the invention has at least one disulphide bond which is not present in human insulin.

Disulfide bonds are derived by the coupling of two thiol groups and are herein to be understood as the linkage between two sulfur atoms, i.e. a structure having the overall connectivity R-S-S-R. Disulfide bonds may also be called connecting disulfide bonds, SS-bonds or disulfide bridges. A disulfide bond is created by the introduction of two cysteine amino acid residues to a peptide with subsequent oxidation of the two thiol groups to a disulfide bond. Such oxidation can be performed chemically (as known by persons skilled in the art) or can happen during insulin expression in e.g. yeast.

When introducing new cysteine residues into the peptide part of the N-terminally modified insulin, the cysteine residues are placed in the three dimensional structure of the folded insulin analogue to allow for the formation of one or more additional disulfide bonds not present in human insulin. For example, if placing two new cysteine residues, the proximity of the new cysteine residues in the three dimensional structure is such that a disulfide bond can be formed between the two new cysteine residues.

The number of disulfide bonds in a protein (such as insulin) can be readily determined by accurate intact mass measurements as described, for example in example 115. The disulfide bonds connectivity can be verified (determined) by standard techniques known in the art, such as peptide mapping. The general strategy for disulfide bond mapping in an insulin peptide includes the following steps: 1) Fragmentation of the non-reduced insulin into disulfide bonded peptides containing, if possible, only a single disulfide bond per peptide. The chosen conditions is also such that rearrangement of disulfide bonds is avoided, 2) separation of disulfide bonded peptides from each other, and 3) identification of the cysteine residues involved in the individual disulfide bonds.

Human insulin is typically digested by Glu-C protease yielding peptide I containing two disulfide bonds (A6-A11 and A7-B7) and peptide II containing a single disulfide bond (A20-B19). To unambiguously assign the disulfide bonds in peptide I, further fragmentation is necessary. Acid hydrolysis (Ryle at al., 1955 Biochem J. 60, 541-56), manual Edman degradation (Kumazaki T, Ishii, S. 1990 J. Biochem (Tokyo) 17, 414-9). or prolonged digestion with thermolysin (Ota M, Ariyoshi, Y., 1995, Biosci. Biotech. Biochem. 59, 1956-7) were previously employed to hydrolyze CysCys bonds in proteins. An alternative way to assign the disulfide bonds in peptide I is a partial reduction with triscarboxyethyl phosphine (reduction of A7-B7 disulfide bond), alkylation of the reduced cysteine residues followed by complete reduction and cysteine alkylation using a different alkyl group (Yen, T.-Y., Yan, H., Macher, B., 2001 J Mass Spectrom. 37, 15-30). The strategy for disulfide mapping of insulins containing extra disulfide bonds is in principle the same as outline above for human insulin adjusted for each analogue in such a way that accommodates the new disulfide bond. Determination of insulin structure by NMR or X-ray crystallography is an alternative approach for verifying the disulfide bond connectivity. Conditions for solving NMR and/or X-ray structures of insulin have been described previously and are known in the art.

In one aspect of the invention an N-terminally modified insulin which has an N-terminal modification, an albumin binding moiety and at least two cysteine substitutions is provided, where the three disulfide bonds of human insulin are retained.

With the term “cysteine substitution” is herein meant replacing an amino acid which is present in human insulin with a cysteine. For example, isoleucine in position 10 in the A chain (IleA10) and glutamine in position 4 of the B chain of human insulin (GlnB4) may each be replaced by a cysteine residue. With the term “other amino acid residue substitution” is herein meant replacing an amino acid which is present in human insulin with an amino acid which is not cysteine.

An additional disulfide bond obtained by the invention may be connecting two cysteines of the same chain, i.e. two cysteines in the A-chain or two cysteines in the B-chain of the insulin, or connecting a cysteine in the A-chain with a cysteine in the B-chain of the insulin. In one aspect, an N-terminally modified insulin according to the invention is obtained, wherein at least one additional disulfide bond is connecting two cysteines in the A-chain or connecting two cysteines in the B-chain. In one aspect, an N-terminally modified insulin according to the invention is obtained, wherein at least one additional disulfide bond is connecting a cysteine in the A-chain with a cysteine in the B-chain.

When used herein the term “additional disulfide bonds” means one or more disulfide bonds which are not present in human insulin.

In one embodiment, an N-terminally modified insulin according to the invention has a peptide part which is selected from the group consisting of the following insulin peptides (where “peptide part” means the part of the N-terminally modified insulin of the invention which does not include the N-terminal modifications and/or the “albumin binding moiety” or acyl moiety):

A10C, A14E, B1C, B16H, B25H, desB30 human insulin,

A10C, A14E, B1C, B25H, desB30 human insulin,

A10C, A14E, B2C, B16H, B25H, desB30 human insulin,

A10C, A14E, B2C, B25H, desB30 human insulin,

A10C, A14E, B3C, B16H, B25H, desB30 human insulin,

A10C, A14E, B3C, B25H, desB27, desB30 human insulin,

A10C, A14E, B3C, B25H, desB30 human insulin,

A10C, A14E, B3C, desB27, desB30 human insulin,

A10C, A14E, B3C, B16H, B25H, desB27, desB30 human insulin,

A10C, A14E, B3C, B16H, desB27, desB30 human insulin,

A10C, A14E, B4C, B16H, desB27, desB30 human insulin

A10C, A14E, B4C, B16H, B25H, desB30 human insulin,

A10C, A14E, B4C, B25A, desB30 human insulin,

A10C, A14E, B4C, B25H, B28E, desB30 human insulin,

A10C, A14E, B4C, B25H, desB27, desB30 human insulin,

A10C, A14E, B4C, B25H, desB30 human insulin,

A10C, A14E, B4C, B25N, B27E, desB30 human insulin,

A10C, A14E, B4C, B25N, desB27, desB30 human insulin,

A10C, A14E, desB1, B4C, B25H, desB30 human insulin,

A10C, A14H, B4C, B25H, desB30 human insulin,

A10C, A14E, B1C, B16H, B25H, desB30 human insulin,

A10C, A14E, B2C, B16H, B25H, desB30 human insulin,

A10C, A14E, B3C, B16H, B25H, desB30 human insulin,

A10C, A14E, B4C, B16H, B25H, desB30 human insulin

In one embodiment, an N-terminally modified insulin according to the invention has a peptide part which is selected from the group consisting of the following insulin peptides:

A10C, A14E, B1C, B16H, B25H, desB30 human insulin,

A10C, A14E, B1C, B25H, desB30 human insulin,

A10C, A14E, B2C, B16H, B25H, desB30 human insulin,

A10C, A14E, B2C, B25H, desB30 human insulin,

A10C, A14E, B3C, B16H, B25H, desB30 human insulin,

A10C, A14E, B3C, B25H, desB27, desB30 human insulin,

A10C, A14E, B3C, B25H, desB30 human insulin,

A10C, A14E, B3C, desB27, desB30 human insulin,

A10C, A14E, B3C, B16H, B25H, desB27, desB30 human insulin,

A10C, A14E, B3C, B16H, desB27, desB30 human insulin,

A10C, A14E, B4C, B16H, desB27, desB30 human insulin,

A10C, A14E, B4C, B16H, B25H, desB30 human insulin,

A10C, A14E, B4C, B25A, desB30 human insulin,

A10C, A14E, B4C, B25H, B28E, desB30 human insulin,

A10C, A14E, B4C, B25H, desB27, desB30 human insulin,

A10C, A14E, B4C, B25H, desB30 human insulin,

A10C, A14E, B4C, B25N, B27E, desB30 human insulin,

A10C, A14E, B4C, B25N, desB27, desB30 human insulin,

A10C, A14E, desB1, B4C, B25H, desB30 human insulin,

A10C, A14H, B4C, B25H, desB30 human insulin,

A10C, A14E, B1C, B16H, B25H, desB30 human insulin,

A10C, A14E, B2C, B16H, B25H, desB30 human insulin,

A10C, A14E, B3C, B16H, B25H, desB30 human insulin,

A10C, A14E, B4C, B16H, B25H, desB30 human insulin

The term “human insulin” as used herein means the human insulin hormone whose two dimensional and three dimensional structures and properties are well-known. The three dimensional structure of human insulin has been e.g. determined by NMR and X-ray crystallography under many different conditions and many of these structures are deposited in the Protein data bank (http://www.rcsb.org). Non-limiting examples of a human insulin structure is the T6 structure (http://www.rcsb.org/pdb/explore.do?structureId=1MSO) and the R6 structure (http:www.rcsb.org/pdb/explore.do?structureId=1EV3). Human insulin has two polypeptide chains, named the A-chain and the B-chain. The A-chain is a 21 amino acid peptide and the B-chain is a 30 amino acid peptide, the two chains being connected by disulfide bonds: a first bridge between the cysteine in position 7 of the A-chain and the cysteine in position 7 of the B-chain, and a second bridge between the cysteine in position 20 of the A-chain and the cysteine in position 19 of the B-chain. A third bridge is present between the cysteines in position 6 and 11 of the A-chain. Thus “a N-terminally modified insulin where the three disulfide bonds of human insulin are retained” is herein understood as a N-terminally modified insulin comprising the three disulfide bonds of human insulin, i.e. a disulfide bond between the cysteine in position 7 of the A-chain and the cysteine in position 7 of the B-chain, a disulfide bond between the cysteine in position 20 of the A-chain and the cysteine in position 19 of the B-chain and a disulfide bond between the cysteines in position 6 and 11 of the A-chain.

In the human body, the insulin hormone is synthesized as a single-chain precursor proinsulin (preproinsulin) consisting of a prepeptide of 24 amino acids followed by proinsulin containing 86 amino acids in the configuration: prepeptide-B-Arg Arg-C-Lys Arg-A, in which C is a connecting peptide of 31 amino acids. Arg-Arg and Lys-Arg are cleavage sites for cleavage of the connecting peptide from the A and B chains.

In one aspect of the invention an N-terminally modified insulin which has two or more cysteine substitutions is provided, where the three disulfide bonds of human insulin are retained, and wherein at least one amino acid residue in a position selected from the group consisting of A9, A10 and A12 of the A-chain is substituted with a cysteine, at least one amino acid residue in a position selected from the group consisting of B1, B2, B3, B4, B5 and B6 of the B-chain is substituted with a cysteine, a side chain is attached to the epsilon amino group of a lysine residue in the B-chain and optionally the amino acid in position B30 is deleted. In one aspect of the invention the amino acid residue in position A10 of the A-chain is substituted with a cysteine, at least one amino acid residue in a position selected from the group consisting of B1, B2, B3, and B4 of the B-chain is substituted with a cysteine, a side chain is attached to the epsilon amino group of a lysine residue in the B-chain and optionally the amino acid in position B30 is deleted. In one aspect of the invention at least one amino acid residue in a position selected from the group consisting of A9, A10 and A12 of the A-chain is substituted with a cysteine, at least one amino acid residue in a position selected from the group consisting of B1, B2, B3, B4, B5 and B6 of the B-chain is substituted with a cysteine, at least one amino acid residue in a position selected from the group consisting of A14, A21, B1, B3, B10, B16, B22, B25, B26, B27, B28, B29, B30, B31, B32 is substituted with an amino acid which is not a cysteine, a side chain is attached to the epsilon amino group of a lysine residue in the B-chain and optionally the amino acid in position B30 is deleted.

It is understood that when B1 or B3 is cysteine, the same amino acid can not be an amino acid which is not cysteine, whereas if e.g. B1 is cysteine B3 may according to the aspect of the invention be substituted with an amino acid which is not a cysteine and vice versa.

In one aspect of the invention, the amino acid residue in position A10 of the A-chain is substituted with a cysteine, at least one amino acid residue in a position selected from the group consisting of B1, B2, B3, and B4 of the B-chain is substituted with a cysteine, optionally at least one amino acid residue is substituted with an amino acid which is not a cysteine, a side chain is attached to the epsilon amino group of a lysine residue in the B-chain and optionally the amino acid in position B30 is deleted. In one aspect of the invention, the amino acid residue in position A10 of the A-chain is substituted with a cysteine, at least one amino acid residue in a position selected from the group consisting of B3 and B4 of the B-chain is substituted with a cysteine, optionally at least one amino acid residue is substituted with an amino acid which is not a cysteine, a side chain is attached to the epsilon amino group of a lysine residue in the B-chain and optionally the amino acid in position B30 is deleted. In one aspect of the invention, the amino acid residue in position A10 of the A-chain is substituted with a cysteine, the amino acid residue in position B3 of the B-chain is substituted with a cysteine, optionally at least one amino acid residue is substituted with an amino acid which is not a cysteine, a side chain is attached to the epsilon amino group of a lysine residue in the B-chain and optionally the amino acid in position B30 is deleted. In one aspect of the invention, the amino acid residue in position A10 of the A-chain is substituted with a cysteine, the amino acid residue in B4 of the B-chain is substituted with a cysteine, optionally at least one amino acid residue is substituted with an amino acid which is not a cysteine, a side chain is attached to the epsilon amino group of a lysine residue in the B-chain and optionally the amino acid in position B30 is deleted.

In one aspect, N-terminally modified insulins according to the invention are obtained, wherein position B30 is deleted.

In one aspect of the invention, cysteines are substituted into two positions of the N-terminally modified insulin, where the positions are selected from the group consisting of:

A10C, B1C;

A10C, B2C;

A10C, B3C;

A10C, B4C;

A10C, B5C; and

B1C, B4C.

In one aspect of the invention, cysteines are substituted into two positions of the insulin analogue, where the positions are selected from the group consisting of:

A10C, B1C;

A10C, B2C;

A10C, B3C;

A10C, B4C; and

B1C, B4C.

In one aspect of the invention, cysteines are substituted into two positions of the N-terminally modified insulin, where the positions are selected from the group consisting of:

A10C, B1C;

A10C, B2C;

A10C, B3C; and

A10C, B4C.

In one aspect of the invention, cysteines are substituted into two positions of the insulin analogue, where the positions are selected from the group consisting of:

A10C, B3C; and

A10C, B4C.

In one aspect of the invention, cysteines are substituted into two positions of the insulin analogue, where the positions are A10C and B3C.

In one aspect of the invention, cysteines are substituted into two positions of the insulin analogue, where the positions are A10C and B4C.

In one aspect of the invention, N-terminally modified insulins of the invention comprise in addition to the cysteine substitutions one or more amino acids selected from the group consisting of: A8H, A14E, A14H, A21G, B1G, B3Q, B3E, B3T, B3V, B3L, B16H, B16E, B25A, B25H, B25N, B27E, B27P, B28E, desB1, desB27 and desB30.

In one aspect of the invention, N-terminally modified insulins of the invention comprise in addition to the cysteine substitutions one or more amino acids selected from the group consisting of: A14E, A14H, A21G, desB1, B1G, B3Q, B3E, B16H, B16E, B25H, desB27, and desB30.

In one aspect of the invention, N-terminally modified insulins of the invention comprise in addition to the cysteine substitutions one or more amino acids selected from the group consisting of: A14E, desB1, B1G, B16H, B16E, B25H, desB27, and desB30.

In one aspect of the invention, N-terminally modified insulins of the invention comprise in addition to the cysteine substitutions one or more amino acids selected from the group consisting of: A14E, B16H, B25H, desB27, and desB30.

Herein, the term “acylated insulin” covers modification of insulin by attachment of one or more albumin binding moietys optionally via a linker to the insulin peptide.

An “albumin binding moiety” is herein understood as a side chain consisting of a fatty acid or a fatty diacid attached to the insulin, optionally via a linker, in an amino acid position such as LysB29, or equivalent.

In one embodiment, the “albumin binding moiety” attached to the N-terminally modified insulin has the general formula:

Acy-AA1ₙ-AA2ₘ-AA3ₚ- (Chem. IV),

wherein n is 0 or an integer in the range from 1 to 3; m is 0 or an integer in the range from 1 to 10; p is 0 or an integer in the range from 1 to 10; Acy is a fatty acid or a fatty diacid comprising from about 14 to about 20 carbon atoms; AA1 is a neutral linear or cyclic amino acid residue; AA2 is an acidic amino acid residue; AA3 is a neutral, alkyleneglycol-containing amino acid residue; the order by which AA1, AA2 and AA3 appears in the formula can be interchanged independently; AA2 can occur several times along the formula (e.g., Acy-AA2-AA3₂-AA2-); AA2 can occur independently (=being different) several times along the formula (e.g., Acy-AA2-AA3₂-AA2-); the connections between Acy, AA1, AA2 and/or AA3 are amide (peptide) bonds which, formally, can be obtained by removal of a hydrogen atom or a hydroxyl group (water) from each of Acy, AA1, AA2 and AA3; and attachment to the peptide part can be from the C-terminal end of a AA1, AA2, or AA3 residue in the acyl moiety of the Chem. IV or from one of the side chain(s) of an AA2 residue present in the moiety of Chem. IV.

A non-limiting example of albumin binding moietys which may be used according to the invention may e.g. be found in the patent application WO 2009/115469, including as the albumin binding moietys of the acylated polypeptides as described in the passage beginning on page 25, line 3 of WO 2009/115469.

In one aspect of the invention, albumin binding moiety is selected from the group consisting of:

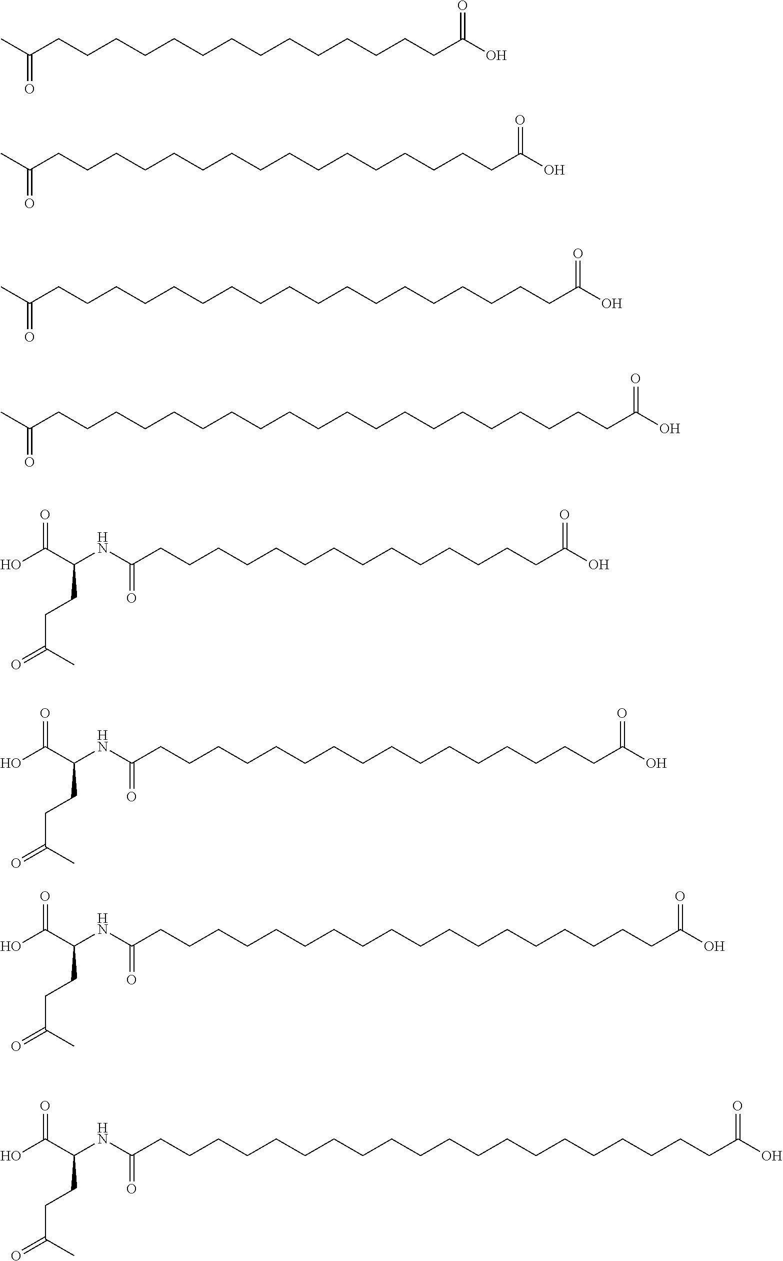

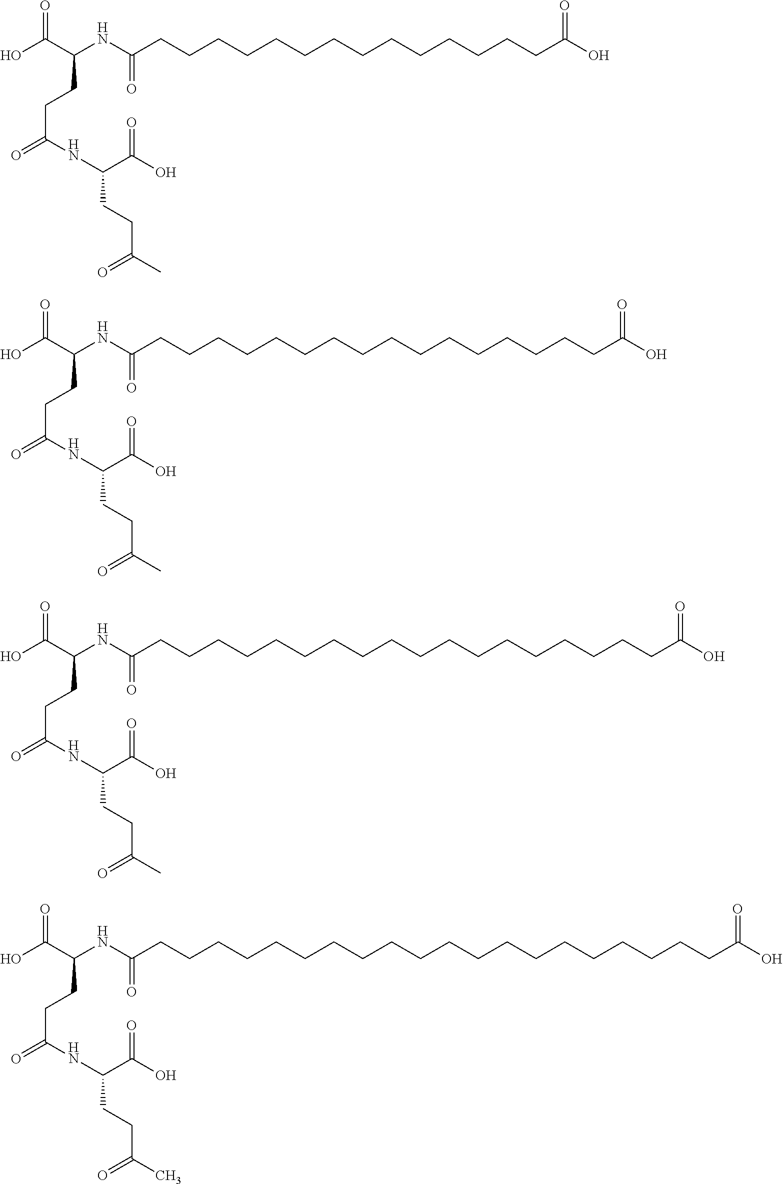 [SMILES: CC(=O)CC[C@H](NC(=O)CC[C@H](NC(=O)CCCCCCCCCCCCCCC(=O)O)C(=O)O)C(=O)O ; CC(=O)CC[C@H](NC(=O)CC[C@H](NC(=O)CCCCCCCCCCCCCCCCC(=O)O)C(=O)O)C(=O)O ; CC(=O)CC[C@H](NC(=O)CC[C@H](NC(=O)CCCCCCCCCCCCCCCCCCC(=O)O)C(=O)O)C(=O)O ; CC(=O)CC[C@H](NC(=O)CC[C@H](NC(=O)CCCCCCCCCCCCCCCCCCCCC(=O)O)C(=O)O)C(=O)O]

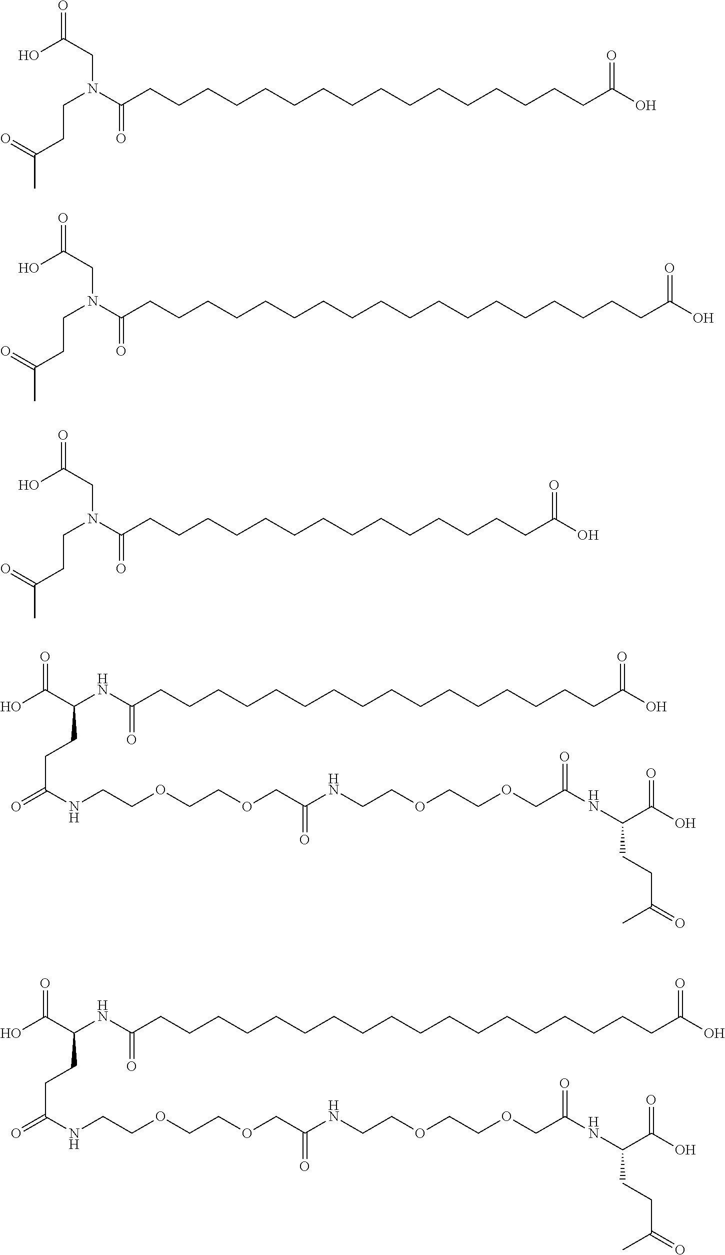

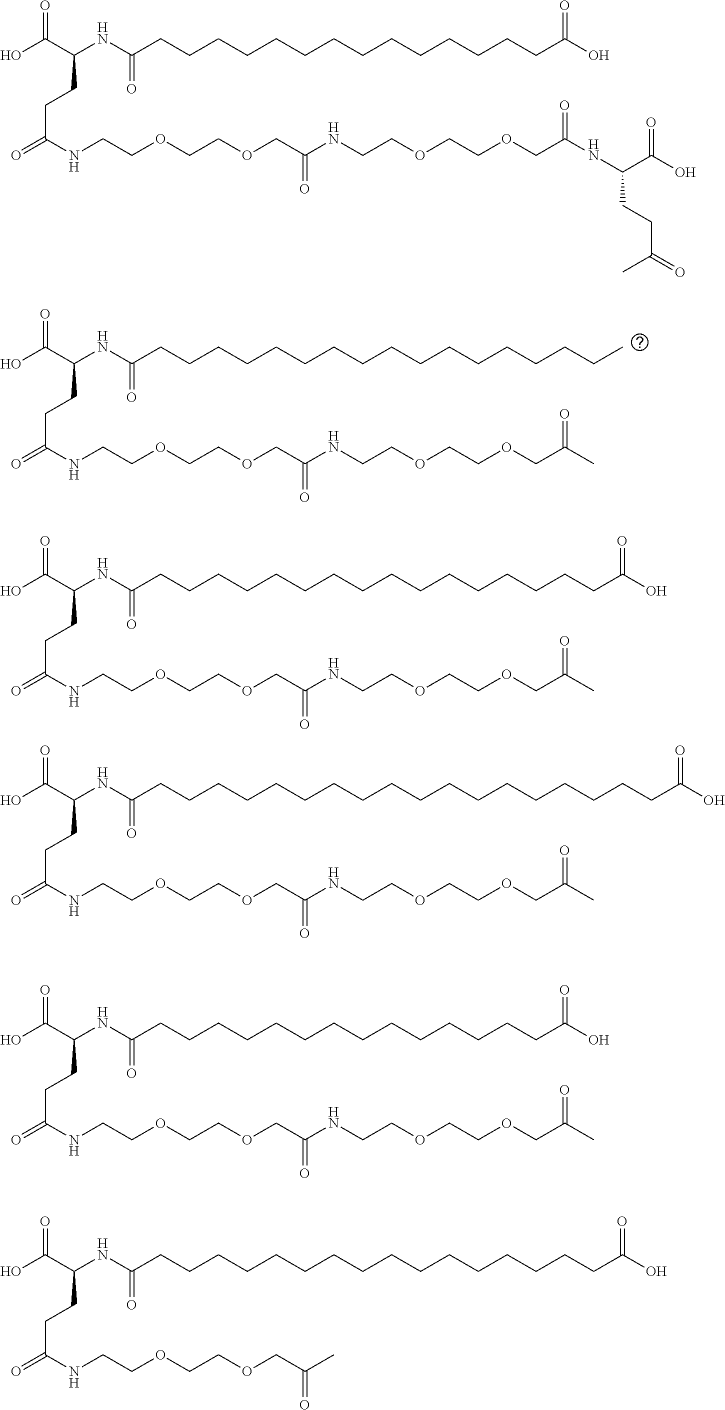 [SMILES: CC(=O)CC[C@H](NC(=O)COCCOCCNC(=O)COCCOCCNC(=O)CC[C@H](NC(=O)CCCCCCCCCCCCCCC(=O)O)C(=O)O)C(=O)O ; CCCCCCCCCCCCCCCCCC(=O)N[C@@H](CCC(=O)NCCOCCOCC(=O)NCCOCCOCC(C)=O)C(=O)O ; CC(=O)COCCOCCNC(=O)COCCOCCNC(=O)CC[C@H](NC(=O)CCCCCCCCCCCCCCCCC(=O)O)C(=O)O ; CC(=O)COCCOCCNC(=O)COCCOCCNC(=O)CC[C@H](NC(=O)CCCCCCCCCCCCCCCCCCC(=O)O)C(=O)O ; CC(=O)COCCOCCNC(=O)COCCOCCNC(=O)CC[C@H](NC(=O)CCCCCCCCCCCCCCC(=O)O)C(=O)O ; CC(=O)COCCOCCNC(=O)CC[C@H](NC(=O)CCCCCCCCCCCCCCCCC(=O)O)C(=O)O]

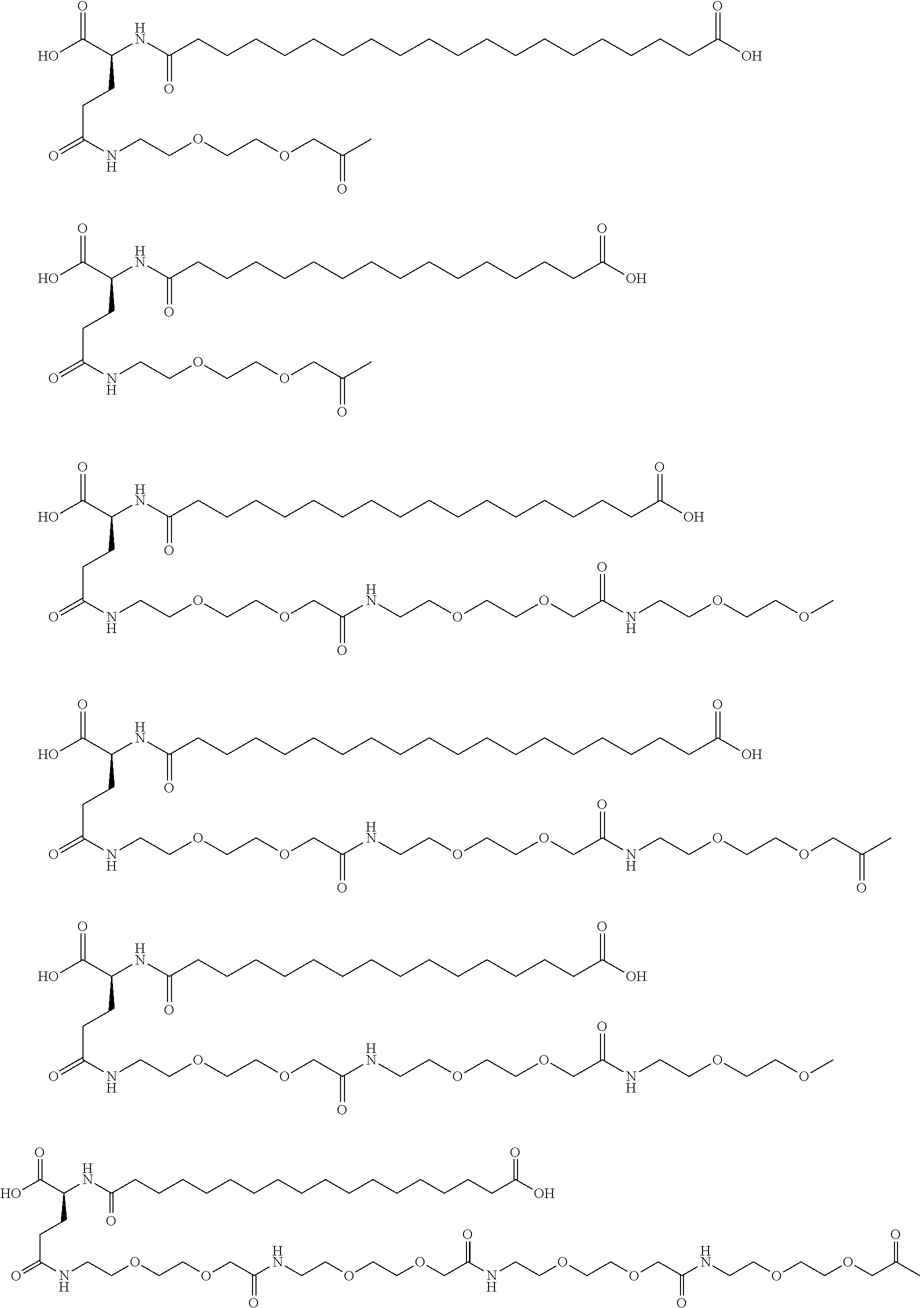 [SMILES: CC(=O)COCCOCCNC(=O)CC[C@H](NC(=O)CCCCCCCCCCCCCCCCCCC(=O)O)C(=O)O ; CC(=O)COCCOCCNC(=O)CC[C@H](NC(=O)CCCCCCCCCCCCCCC(=O)O)C(=O)O ; COCCOCCNC(=O)COCCOCCNC(=O)COCCOCCNC(=O)CC[C@H](NC(=O)CCCCCCCCCCCCCCCCC(=O)O)C(=O)O ; CC(=O)COCCOCCNC(=O)COCCOCCNC(=O)COCCOCCNC(=O)CC[C@H](NC(=O)CCCCCCCCCCCCCCCCCCC(=O)O)C(=O)O ; COCCOCCNC(=O)COCCOCCNC(=O)COCCOCCNC(=O)CC[C@H](NC(=O)CCCCCCCCCCCCCCC(=O)O)C(=O)O ; CC(=O)COCCOCCNC(=O)COCCOCCNC(=O)COCCOCCNC(=O)COCCOCCNC(=O)CC[C@H](NC(=O)CCCCCCCCCCCCCCCCC(=O)O)C(=O)O]

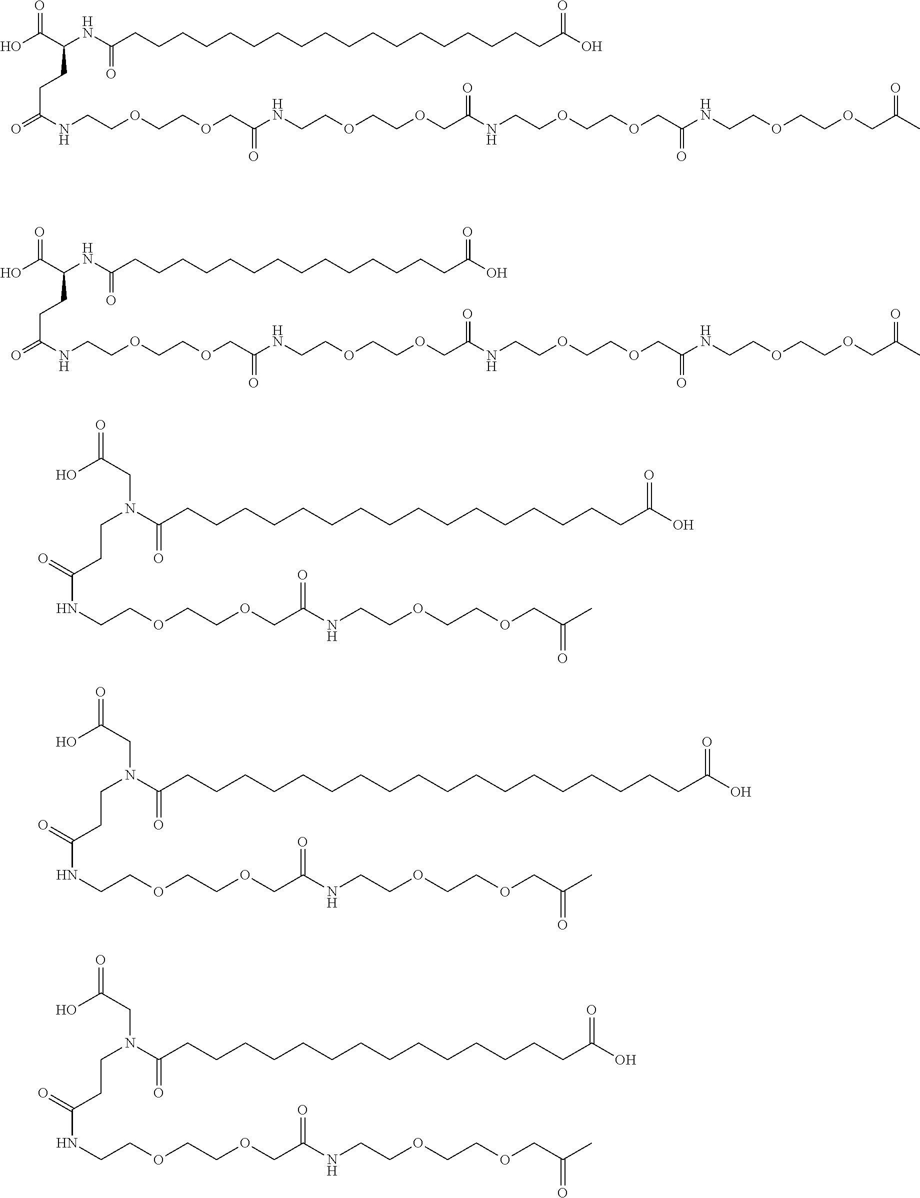 [SMILES: CC(=O)COCCOCCNC(=O)COCCOCCNC(=O)COCCOCCNC(=O)COCCOCCNC(=O)CC[C@H](NC(=O)CCCCCCCCCCCCCCCCCCC(=O)O)C(=O)O ; CC(=O)COCCOCCNC(=O)COCCOCCNC(=O)COCCOCCNC(=O)COCCOCCNC(=O)CC[C@H](NC(=O)CCCCCCCCCCCCCCC(=O)O)C(=O)O ; CC(=O)COCCOCCNC(=O)COCCOCCNC(=O)CCN(CC(=O)O)C(=O)CCCCCCCCCCCCCCCCC(=O)O ; CC(=O)COCCOCCNC(=O)COCCOCCNC(=O)CCN(CC(=O)O)C(=O)CCCCCCCCCCCCCCCCCCC(=O)O ; CC(=O)COCCOCCNC(=O)COCCOCCNC(=O)CCN(CC(=O)O)C(=O)CCCCCCCCCCCCCCC(=O)O]

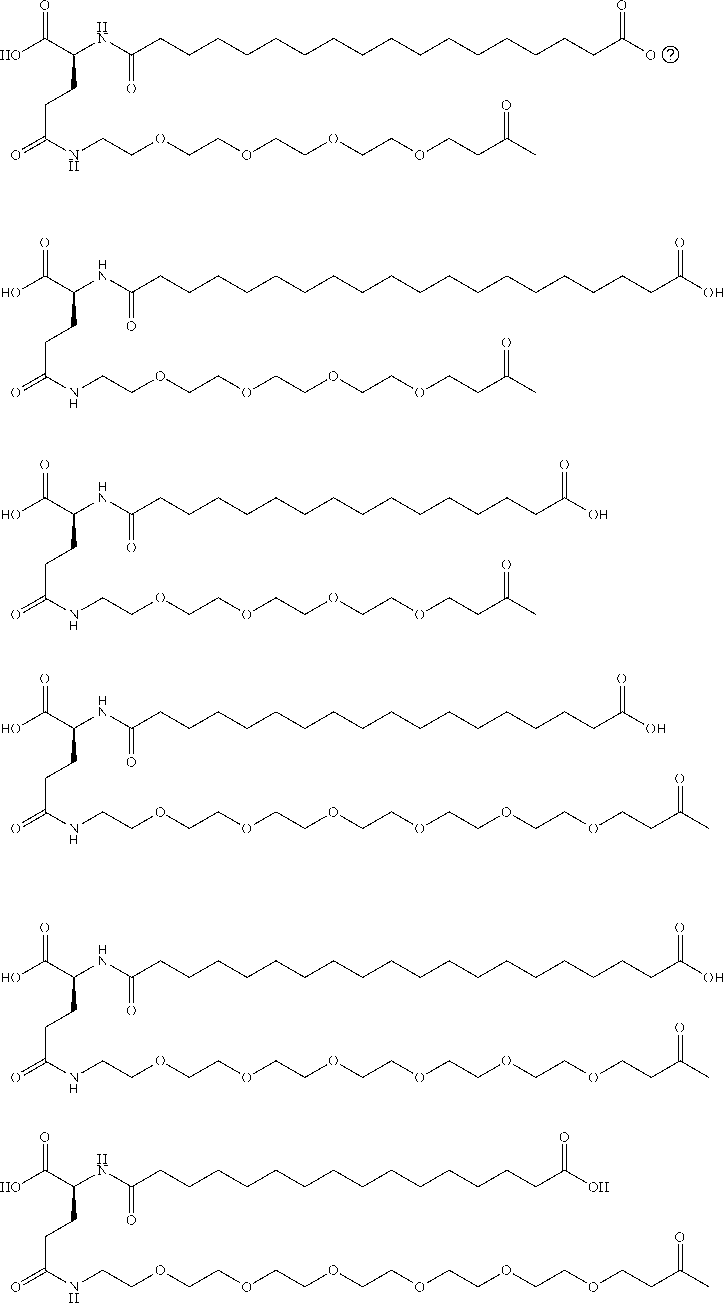 [SMILES: CC(=O)CCOCCOCCOCCOCCNC(=O)CC[C@H](NC(=O)CCCCCCCCCCCCCCCCC(=O)O)C(=O)O ; CC(=O)CCOCCOCCOCCOCCNC(=O)CC[C@H](NC(=O)CCCCCCCCCCCCCCCCCCC(=O)O)C(=O)O ; CC(=O)CCOCCOCCOCCOCCNC(=O)CC[C@H](NC(=O)CCCCCCCCCCCCCCC(=O)O)C(=O)O ; CC(=O)CCOCCOCCOCCOCCOCCOCCNC(=O)CC[C@H](NC(=O)CCCCCCCCCCCCCCCCC(=O)O)C(=O)O ; CC(=O)CCOCCOCCOCCOCCOCCOCCNC(=O)CC[C@H](NC(=O)CCCCCCCCCCCCCCCCCCC(=O)O)C(=O)O ; CC(=O)CCOCCOCCOCCOCCOCCOCCNC(=O)CC[C@H](NC(=O)CCCCCCCCCCCCCCC(=O)O)C(=O)O]

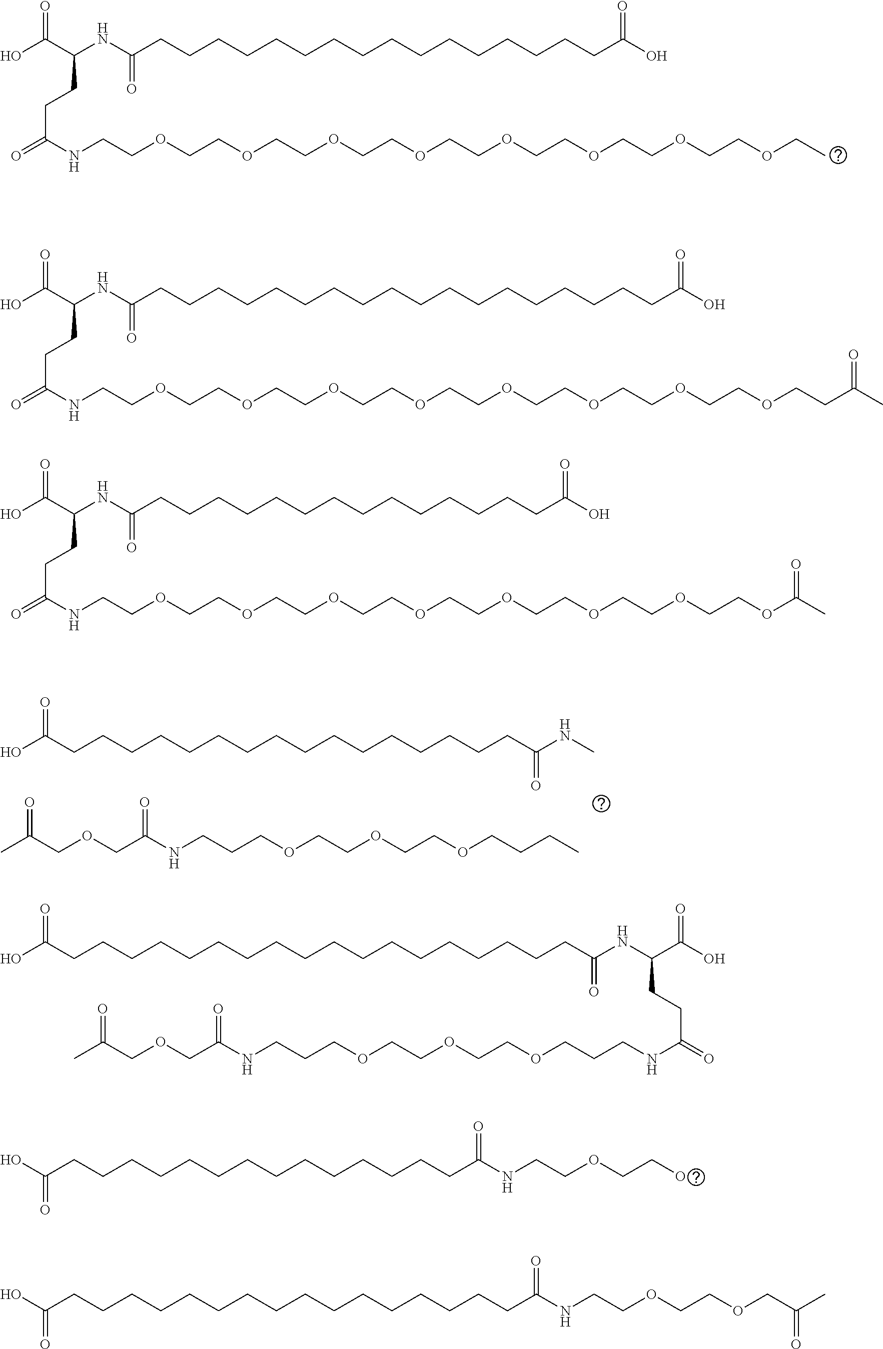

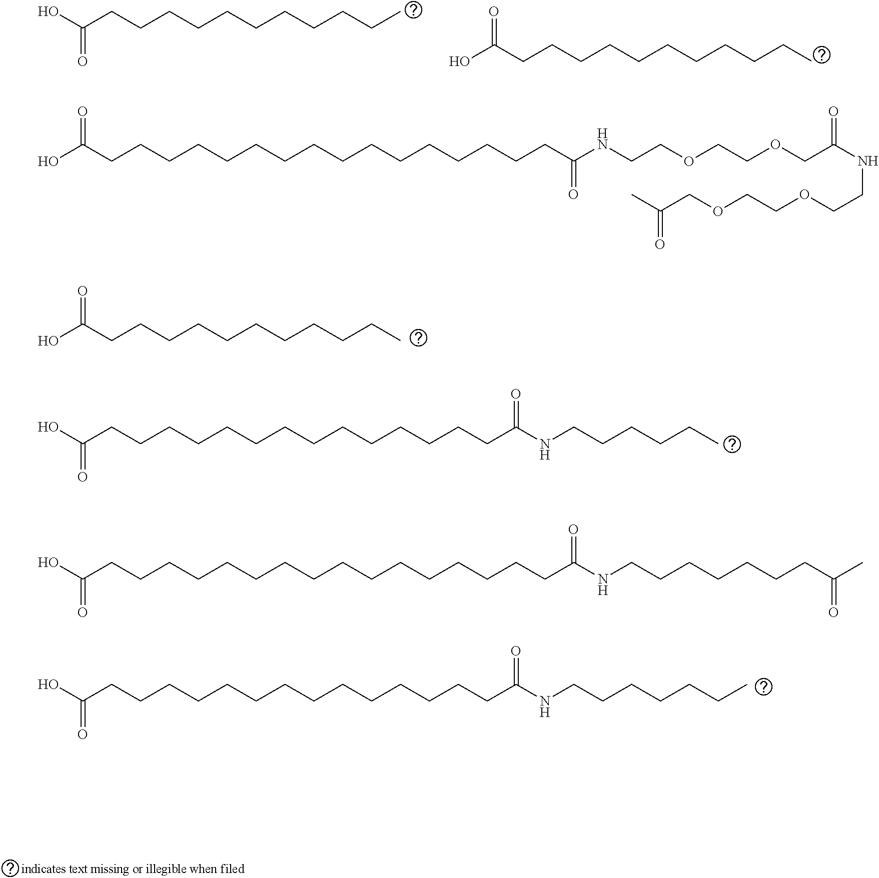

In one aspect of the invention, albumin binding moiety is selected from the group consisting of:

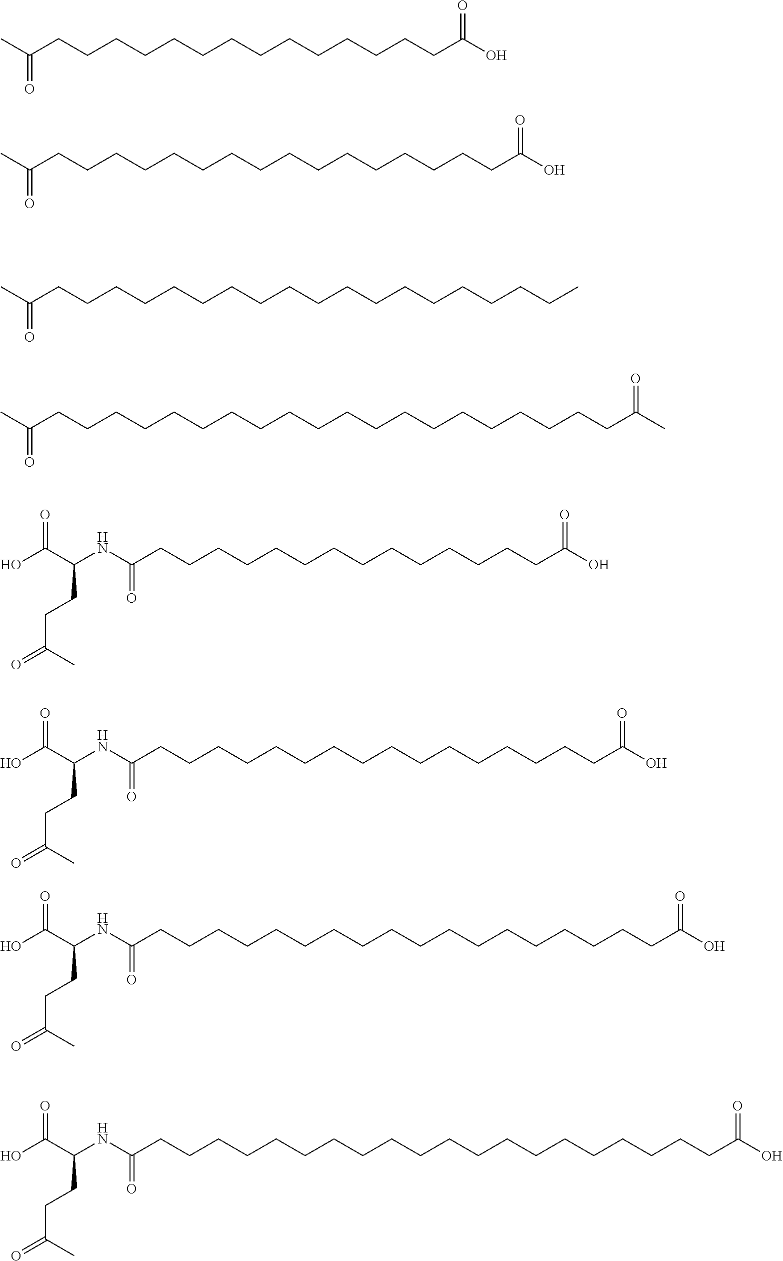

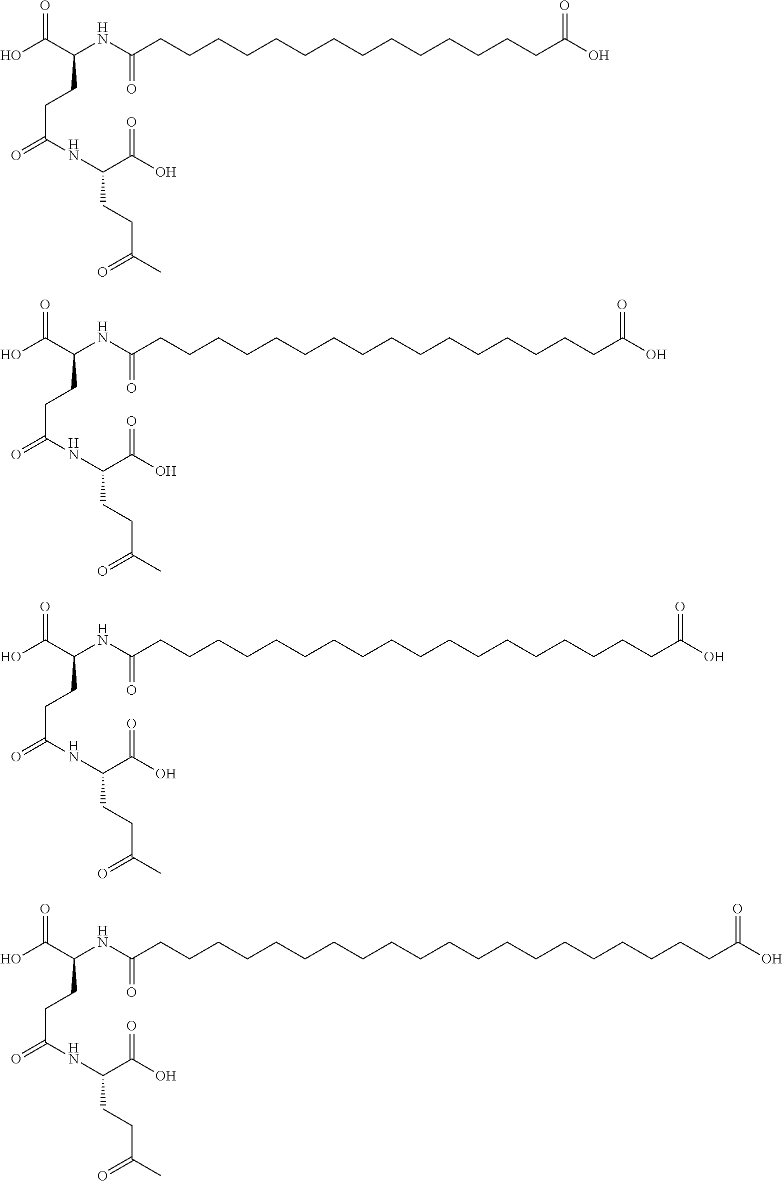 [SMILES: CC(=O)CC[C@H](NC(=O)CC[C@H](NC(=O)CCCCCCCCCCCCCCC(=O)O)C(=O)O)C(=O)O ; CC(=O)CC[C@H](NC(=O)CC[C@H](NC(=O)CCCCCCCCCCCCCCCCC(=O)O)C(=O)O)C(=O)O ; CC(=O)CC[C@H](NC(=O)CC[C@H](NC(=O)CCCCCCCCCCCCCCCCCCC(=O)O)C(=O)O)C(=O)O ; CC(=O)CC[C@H](NC(=O)CC[C@H](NC(=O)CCCCCCCCCCCCCCCCCCCCC(=O)O)C(=O)O)C(=O)O]

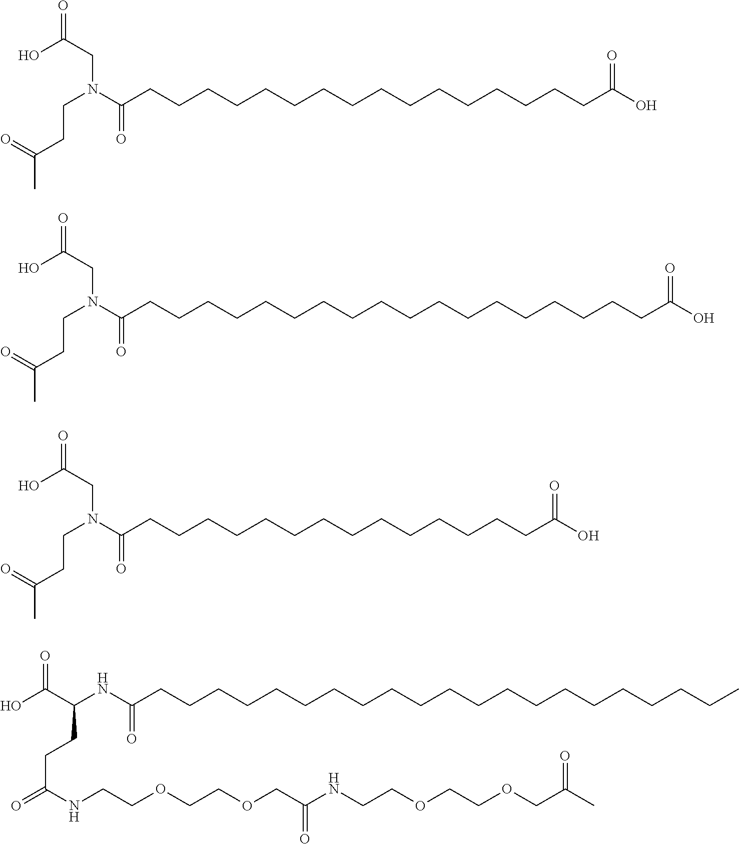

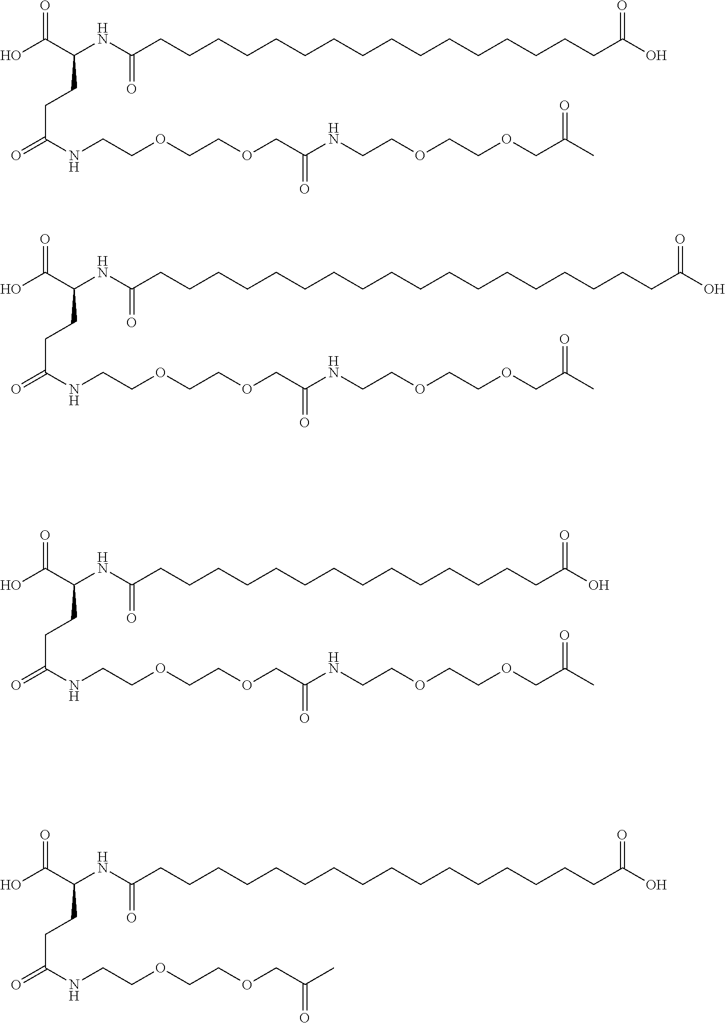 [SMILES: CC(=O)COCCOCCNC(=O)COCCOCCNC(=O)CC[C@H](NC(=O)CCCCCCCCCCCCCCCCC(=O)O)C(=O)O ; CC(=O)COCCOCCNC(=O)COCCOCCNC(=O)CC[C@H](NC(=O)CCCCCCCCCCCCCCCCCCC(=O)O)C(=O)O ; CC(=O)COCCOCCNC(=O)COCCOCCNC(=O)CC[C@H](NC(=O)CCCCCCCCCCCCCCC(=O)O)C(=O)O ; CC(=O)COCCOCCNC(=O)CC[C@H](NC(=O)CCCCCCCCCCCCCCCCC(=O)O)C(=O)O]

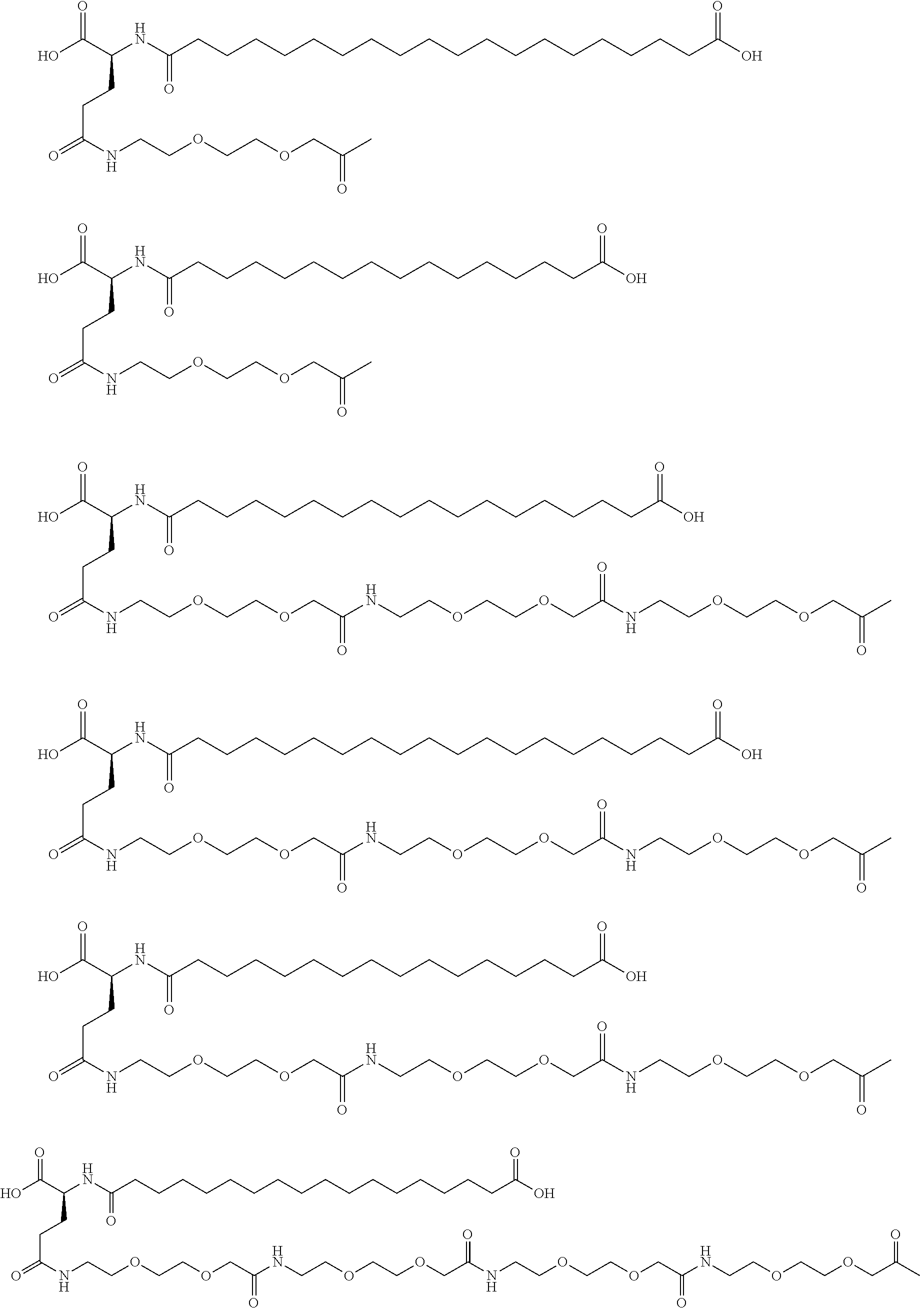

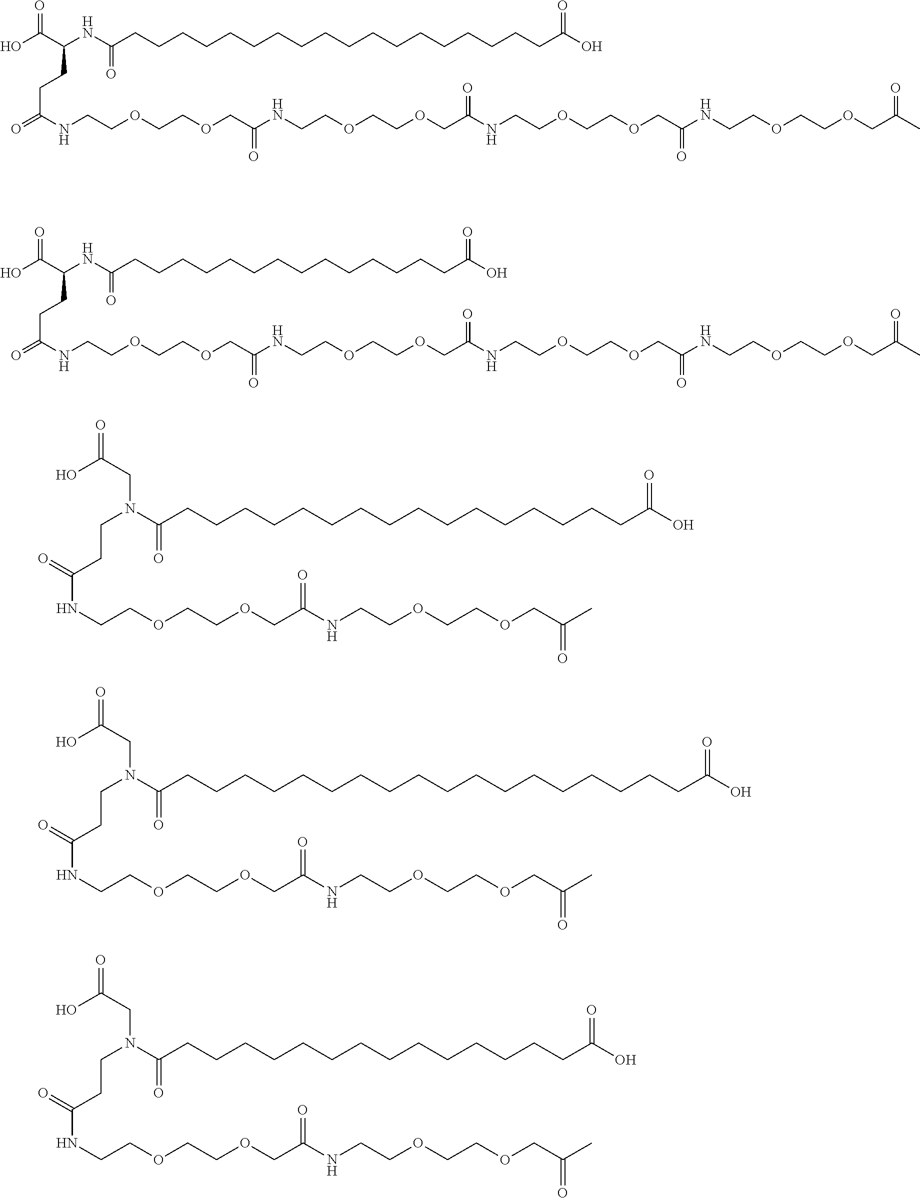 [SMILES: CC(=O)COCCOCCNC(=O)COCCOCCNC(=O)COCCOCCNC(=O)COCCOCCNC(=O)CC[C@H](NC(=O)CCCCCCCCCCCCCCCCCCC(=O)O)C(=O)O ; CC(=O)COCCOCCNC(=O)COCCOCCNC(=O)COCCOCCNC(=O)COCCOCCNC(=O)CC[C@H](NC(=O)CCCCCCCCCCCCCCC(=O)O)C(=O)O ; CC(=O)COCCOCCNC(=O)COCCOCCNC(=O)CCN(CC(=O)O)C(=O)CCCCCCCCCCCCCCCCC(=O)O ; CC(=O)COCCOCCNC(=O)COCCOCCNC(=O)CCN(CC(=O)O)C(=O)CCCCCCCCCCCCCCCCCCC(=O)O ; CC(=O)COCCOCCNC(=O)COCCOCCNC(=O)CCN(CC(=O)O)C(=O)CCCCCCCCCCCCCCC(=O)O]

In one aspect of the invention, albumin binding moiety is selected from the group consisting of:

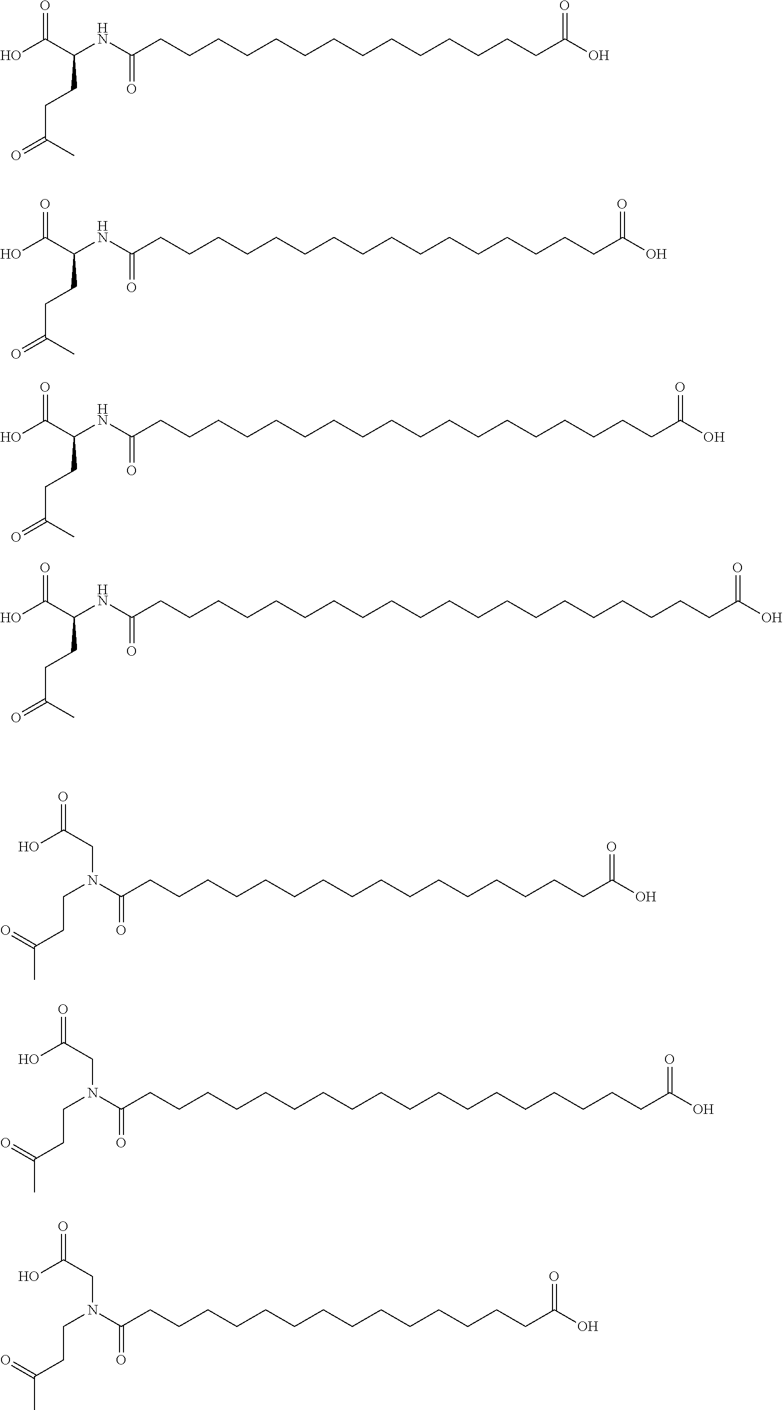

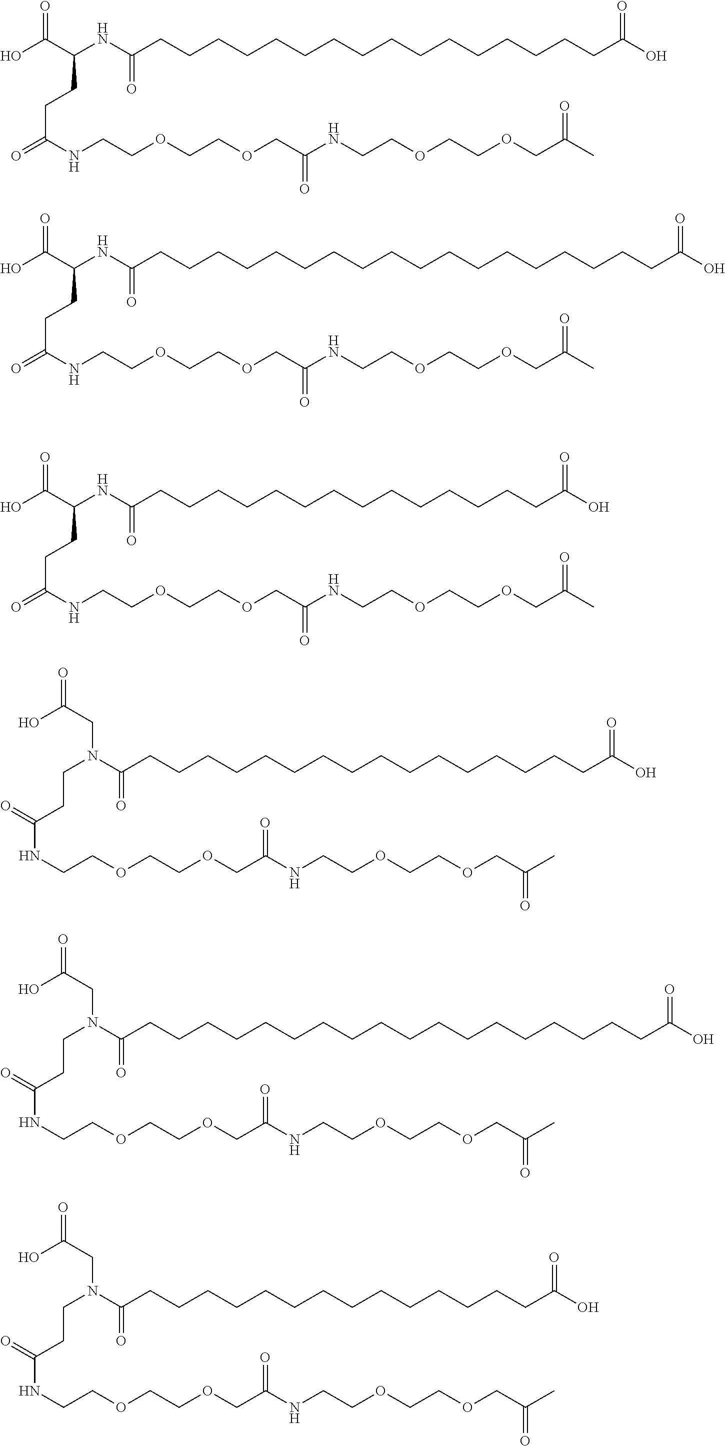 [SMILES: CC(=O)COCCOCCNC(=O)COCCOCCNC(=O)CC[C@H](NC(=O)CCCCCCCCCCCCCCCCC(=O)O)C(=O)O ; CC(=O)COCCOCCNC(=O)COCCOCCNC(=O)CC[C@H](NC(=O)CCCCCCCCCCCCCCCCCCC(=O)O)C(=O)O ; CC(=O)COCCOCCNC(=O)COCCOCCNC(=O)CC[C@H](NC(=O)CCCCCCCCCCCCCCC(=O)O)C(=O)O ; CC(=O)COCCOCCNC(=O)COCCOCCNC(=O)CCN(CC(=O)O)C(=O)CCCCCCCCCCCCCCCCC(=O)O ; CC(=O)COCCOCCNC(=O)COCCOCCNC(=O)CCN(CC(=O)O)C(=O)CCCCCCCCCCCCCCCCCCC(=O)O ; CC(=O)COCCOCCNC(=O)COCCOCCNC(=O)CCN(CC(=O)O)C(=O)CCCCCCCCCCCCCCC(=O)O]

An“N-terminally modified insulin” is herein the same as an “N-terminally protected insulin” and is herein defined as an insulin comprising one or more N-terminal modification groups also herein named N-terminal protecting groups.

“N-terminal modification groups” are herein the same as “N-terminal protecting groups” and according to the invention are groups that, when conjugated to the N-terminal amino groups of the A- and/or B-chain of the insulin, protect said amino groups of the N-terminal amino acids of the insulin (typically, but not always), glycine and phenylalanine of the A- and the B-chain, respectively, from reacting with e.g. aldehyde impurities of one or more of the excipients in a pharmaceutical formulation. In one aspect of the invention the N-terminal modification is one or two organic substituents having a MW below 200 g per mol conjugated to an N-terminal of the parent insulin”.

In one aspect the N-terminally modified insulin of the invention comprises the N-terminal modification groups Y′ and Z attached to at least one, preferably two N-terminal amino acid(s) as illustrated in Chem. V with the first four residues of the insulin A-chain N-terminal shown (GIVE . . . ).

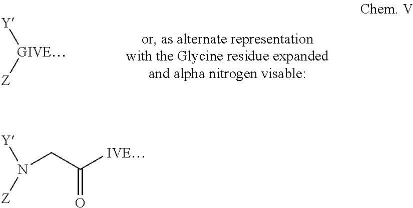

In one aspect of the invention, Y′ and Z are different and:
Y′ is R—C(═X)—,
Z is H,
R is H, NH₂, straight chain or branched C1-C4 alkyl, (optionally substituted with dimethylamino, diethylamino, dipropylamino, trimethylammonium, triethylammonium, or tripropylammonium), C5-C6 cycloalkyl (optionally substituted), 5- or 6 membered saturated heterocyclyl (optionally substituted), and
X is O or S.

In one aspect of the invention, when Y′ is R—C(═X)— and Z is H, the insulin can contain the desA1 and desB1 mutations.

In another aspect of the invention, Y′=Z is C1-C4 alkyl.

In one aspect of the invention, each of the N-terminal protecting groups of the A- and the B-chain N-terminal amino groups are the same.

In one aspect of the invention, each of the two N-terminal protecting groups of the invention is having a molecular weight below 150 Da.

In one aspect of the invention, each of the N-terminal protecting groups of the invention is positively charged at physiological pH, i.e. when the N-terminal modification group is attached/conjugated to the N-terminal amino group, the amino group, or the substituent on the amino group, has a positive charge. In one aspect of the invention, the N-terminal protecting groups are selected from the group consisting of: Dimethyl, diethyl, di-n-propyl, di-sec-propyl, di-n-butyl, di-i-butyl or the like. In another aspect of the invention, the N-terminal protecting groups are selected from dimethyl and diethyl. In another aspect of the invention, the N-terminal protecting group is dimethyl.

In one aspect of the invention, the N-terminal protecting groups are selected from the group consisting of: N,N-Dimethylglycyl, N,N-dimethylaminobutanoyl, N,N-dimethylaminopropionyl and 3-(1-piperidinyl)propionyl.

In one aspect of the invention, each of the N-terminal protecting groups of the invention removes the normal positive (or partly positive) charge of the N-terminal amino groups at physiological pH. In one aspect of the invention, each of the N-terminal protecting groups of the invention is selected from small acyl residues. In one aspect of the invention, each of the N-terminal protecting groups of the invention is selected from formyl, acetyl, propanoyl, and butanoyl groups. In one aspect of the invention, each of the N-terminal protecting groups of the invention is selected from cyclic acyl residues, e.g. the pyroglutaminyl (=5-oxo-pyrrolidine-2-oyl) group.

In one aspect of the invention, each of the N-terminal protecting groups of the invention removes the normal positive (or partly positive) charge of the N-terminal amino groups at physiological pH. In one aspect of the invention, each of the N-terminal protecting groups of the invention is selected from carbamoyl and thiocarbamoyl. In one aspect of the invention, each of the N-terminal protecting groups of the invention is carbamoyl.

In one aspect of the invention, each of the N-terminal protecting groups of the invention removes the normal positive (or partly positive) charge of the N-terminal amino groups at physiological pH. In one aspect of the invention, each of the N-terminal protecting groups of the invention is selected from oxalyl, glutaryl, or diglycolyl (other names: 3-oxaglutaryl, carboxymethoxyacetyl). In one aspect of the invention, each of the N-terminal protecting groups of the invention is selected from glutaryl and diglycolyl (other names: 3-oxaglutaryl, carboxymethoxyacetyl). In one aspect of the invention, each of the N-terminal protecting groups of the invention is glutaryl. In one aspect of the invention, each of the N-terminal protecting groups of the invention is diglycolyl (other names: 3-oxaglutaryl, carboxymethoxyacetyl).

When used herein, the term “conjugate” is intended to indicate the process of bonding a substituent to a polypeptide to modify the properties of said polypeptide. “Conjugation” or a “conjugation product” of a molecule and a polypeptide is thus a term for said substituent bonded to an amino acid of the polypeptide and a “substituent” as described herein thus means the substituent which is attached to the polypeptide.

“Monoalkylation” is herein to be understood as conjugation of one alkyl substituent to a free amino group of a polypeptide and “dialkylation” is to be understood as conjugation of two alkyl substituents to a free amino group of a polypeptide as illustrated below, where a “free amino group” is to be understood as a primary amine, R—NH2, or a secondary amine, R1-NH—R2, where R, R1 and R2 represents a substituent.

“Guadinylation” is herein to be understood as conjugation of an amidinyl substituent (which may also be referred to as carboxamidine, i.e. a substitutent of the form: RₙC(═NR)NR₂, where Rₙ is the polypeptide) to a free amino group of the polypeptide resulting in transformation of the amino group to a guadinyl group as illustrated below.

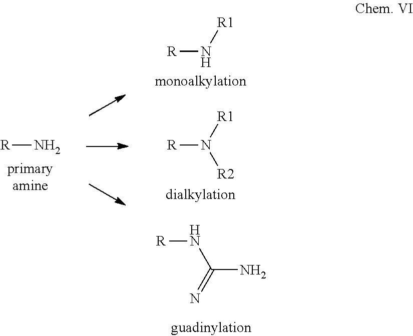

An “insulin analogue” as used herein is a polypeptide which has a molecular structure which formally can be derived from the structure of a naturally occurring insulin, for example that of human insulin, by deleting and/or substituting at least one amino acid residue occurring in the natural insulin and/or by adding at least one amino acid residue.

In one aspect an insulin analogue according to the invention comprises less than 8 modifications (substitutions, deletions, additions) relative to human insulin. In one aspect an insulin analogue comprises less than 7 modifications (substitutions, deletions, additions) relative to human insulin. In one aspect an insulin analogue comprises less than 6 modifications (substitutions, deletions, additions) relative to human insulin. In another aspect an insulin analogue comprises less than 5 modifications (substitutions, deletions, additions) relative to human insulin. In another aspect an insulin analogue comprises less than 4 modifications (substitutions, deletions, additions) relative to human insulin. In another aspect an insulin analogue comprises less than 3 modifications (substitutions, deletions, additions) relative to human insulin. In another aspect an insulin analogue comprises less than 2 modifications (substitutions, deletions, additions) relative to human insulin.

A “derivative of insulin” is a naturally occurring human insulin or an insulin analogue which has been chemically modified, e.g. by introducing a side chain in one or more positions of the insulin backbone or by oxidizing or reducing groups of the amino acid residues in the insulin or by converting a free carboxylic group to an ester group or to an amide group. Other derivatives are obtained by acylating a free amino group or a hydroxy group, such as in the B29 position of human insulin or desB30 human insulin. An insulin molecule comprising an albumin binding moiety is thus an insulin derivative according to this definition.

A derivative of insulin is thus human insulin or an insulin analogue which comprises at least one covalent modification such as a side-chain attached to one or more amino acids of the insulin peptide.

Herein, the naming of the insulins is done according to the following principles: The names are given as mutations and modifications (acylations) relative to human insulin. For the naming of the acyl moiety (albumin binding moiety), the naming is done as peptide nomenclature. For example, naming the acyl moiety:

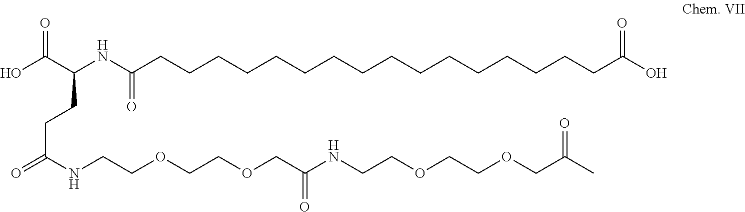 [SMILES: CC(=O)COCCOCCNC(=O)COCCOCCNC(=O)CC[C@H](NC(=O)CCCCCCCCCCCCCCCCC(=O)O)C(=O)O]

can be e.g. “octadecanedioyl-γ-L-Glu-OEG-OEG”, “octadecanedioyl-γ-Glu-2×OEG”, “octadecanedioyl-gGlu-2×OEG”, “17-carboxyheptadecanoyl-γ-L-Glu-OEG-OEG”, or “17-carboxyheptadecanoyl-γ-L-Glu-2×OEG”, wherein

OEG is short hand notation for the amino acid residue —NH(CH₂)₂O(CH₂)₂OCH₂CO—,

γ-L-Glu (alternatively notated g-L-Glu, gGlu, γGlu or gamma-L-Glu) is short hand notation for the L-form of the amino acid gamma glutamic acid moiety.

If the enantiomer form of the gamma glutamic acid moiety is not specified, the moiety may be in the form of a pure enantiomer wherein the stereo configuration of the chiral amino acid moiety is either D or L (or if using the R/S terminology: either R or S) or it may be in the form of a mixture of enantiomers (D and L/R and S).

The acyl moiety of the modified peptides or proteins may be in the form of a pure enantiomer wherein the stereo configuration of the chiral amino acid moiety is either D or L (or if using the R/S terminology: either R or S) or it may be in the form of a mixture of enantiomers (D and L/R and S). In one aspect of the invention the acyl moiety is in the form of a mixture of enantiomers. In one aspect the acyl moiety is in the form of a pure enantiomer. In one aspect the chiral amino acid moiety of the acyl moiety is in the L form. In one aspect the chiral amino acid moiety of the acyl moiety is in the D form.

With “desB30 human insulin” is meant an analogue of human insulin lacking the B30 amino acid residue. With “B1”, “A1” etc. is meant the amino acid residue at position 1 in the B-chain of insulin (counted from the N-terminal end) and the amino acid residue at position 1 in the A-chain of insulin (counted from the N-terminal end), respectively. The amino acid residue in a specific position may also be denoted as e.g. PheB1 or B1F which means that the amino acid residue at position B1 is a phenylalanine residue.

For example, the insulin of example 1 (with the sequence/structure given below) is named “A1(N^{α},N^{α}-Dimethyl), A10C, A14E, B1(N^{α},N^{α}-dimethyl), B3C, B25H, desB27, B29K(N^{ε}octadecanedioyl-gGlu), desB30 human insulin” to indicate that the amino acid in position A10, which is I in human insulin, has been mutated to C, the amino acid in position A14, Y in human insulin, has been mutated to E, the amino acid in position B3, N in human insulin, has been mutated to C, the amino acid in position B25, F in human insulin, has been mutated to H, the amino acids in position A1 and B1 (glycine and phenylalanine, respectively) have been modified by (formally) dimethylation of the N-terminal (alpha) amino groups, and the amino acid in position B27, T in human insulin, has been deleted, the amino acid in position B29, K as in human insulin, has been modified by acylation on the epsilon nitrogen in the lysine residue of B29, denoted N^{ε}, by the residue octadecanedioyl-γGlu, and the amino acid in position B30, T in human insulin, has been deleted. Asterisks in the formula below indicate that the residue in question is different (i.e. mutated) as compared to human insulin. Alternatively, the insulin of example 1 (with the sequence structure given below) can also be named “A1G(N^{α},N^{α}-Dimethyl), A10C, A14E, B1F(N^{α},N^{α}-dimethyl), B3C, B25H, desB27, B29K(N^{ε}octadecanedioyl-gGlu), desB30 human insulin” to further indicate the amino acid residues in position A1 and B1 are G (Gly) and F (Phe), respectively. Furthermore, the notations “N^{α}” and “N^{ε}” can also be written as “N(alpha)” or “N(a)”, and as “N(epsilon)” or “N(eps)”, respectively.

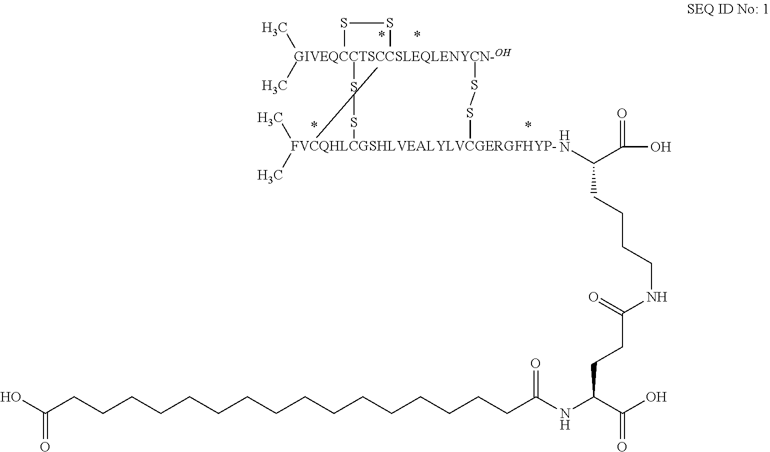

The same insulin may also be illustrated in an alternative representation:

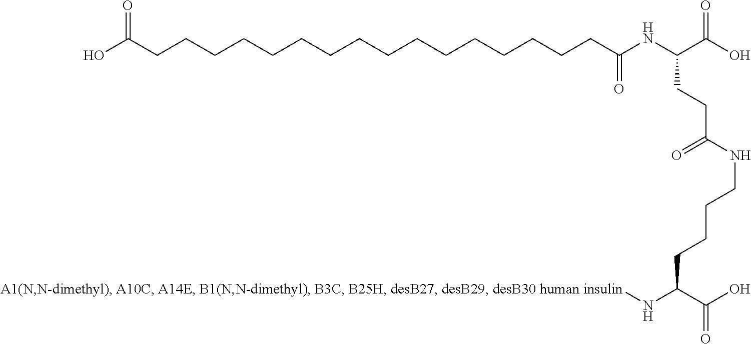

In addition, the insulins of the invention are also named according to IUPAC nomenclature (Open Eye, IUPAC style). According to this nomenclature, the above acylated N-terminally modified insulin is assigned the following name:

N{A1},N{A1}-dimethyl, N{B1},N{B1}-dimethyl, N{Epsilon-B29}-[(4S)-4-carboxy-4-(17-carboxyheptadecanoylamino)butanoyl]-[CysA10,GluA14,CysB3,HisB25], des-ThrB27,ThrB30-Insulin (human)

Notation of N-terminal modifications:

The N-terminal modifications are drawn without the alpha amino group and is to be understood as indicated in the examples below.

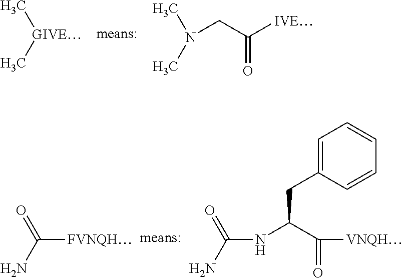

The production of polypeptides is well known in the art. Polypeptides, such as the peptide part of an N-terminal modified insulin according to the invention, may for instance be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. The polypeptides may also be produced by a method which comprises culturing a host cell containing a DNA sequence encoding the polypeptide and capable of expressing the polypeptide in a suitable nutrient medium under conditions permitting the expression of the peptide. For polypeptides comprising non-natural amino acid residues, the recombinant cell should be modified such that the non-natural amino acids are incorporated into the polypeptide, for instance by use of tRNA mutants.

In general, a pharmaceutical composition must be stable during use and storage (in compliance with recommended use and storage conditions) until the expiration date is reached.

In one aspect of the invention a pharmaceutical composition, such as a lipid pharmaceutical composition, comprising an N-terminally modified insulin of the invention is stable for more than 6 weeks of usage and for more than 2 years of storage.

In another aspect of the invention a pharmaceutical composition, such as a lipid pharmaceutical composition, comprising an N-terminally modified insulin of the invention is stable for more than 4 weeks of usage and for more than two years of storage.

In a further aspect of the invention a pharmaceutical composition, such as a lipid pharmaceutical composition, comprising an N-terminally modified insulin of the invention is stable for more than 4 weeks of usage and for more than 3 years of storage.

In an even further aspect of the invention a pharmaceutical composition, such as a lipid pharmaceutical composition, comprising an N-terminally modified insulin of the invention is stable for more than 2 weeks of usage and for more than two years of storage.

In one embodiment, an N-terminally modified insulin of the invention has little or no tendency to aggregate. The aggregation tendency is preferably significantly improved relatively to the aggregation tendency of human insulin and/or the N-terminally modified insulin without one or more additional disulfide bonds when tested in a thioflavin assay. In one aspect the aggregation tendency is improved relatively to the aggregation tendency of the similar acylated insulin with additional disulfide bridge(s) but without the N-terminal modification.

In one aspect, an N-terminally modified insulin according to the invention has improved thermodynamic stability such as e.g. folding stability, conformational stability and/or higher melting temperature.

When used herein an N-terminally modified insulin is said to have improved “thermodynamic stability” if denaturation of said N-terminally modified insulin requires higher stress level such as higher temperature and/or higher concentration of denaturation agent in comparison to human insulin, to an N-terminally modified insulin without one or more additional disulfide bonds or to a similar acylated insulin with additional disulfide bridge(s) but without the N-terminal modification given in similar doses.

Conformational stability may be evaluated by circular dichroism and NMR as e.g. described by Hudson and Andersen, Peptide Science, vol 76 (4), pp. 298-308 (2004). Melting temperature is understood as the temperature at which an insulin structure is reversibly or irreversibly changed. Higher melting temperature corresponds to more stable structures. Melting temperature can be determined e.g. by evaluating conformational stability by circular dichroism and/or NMR as a function of temperature or by differential scanning calorimetry. Thermodynamic stability can also be determined by CD spectroscopy and or NMR in the presence of increasing concentration of denaturation agent, such as for example guanidinium hydrochloride. Free energy of unfolding as described previously (Kaarsholm, N. C., et al, 1993, Biochemistry, 32, 10773-8) can be determined from such experiments. Upon protein denaturation, negative CD in the far UV range (240-218-nm) gradually diminishes, consistent with the loss of ordered secondary structure that accompanies protein unfolding (Holladay et al., 1977, Biochim. Biophys. Acta, 494, 245-254; Melberg and Johnson, 1990, Biochim. Biophys. Acta, 494, 245-254). The insulin CD spectrum in the near UV range (330-250-nm) reflects the environment of the tyrosine chromophore with contributions from the disulfide bonds (Morris et al., 1968, Biochim. Biophys. Acta., 160, 145-155; Wood et al., 1975, Biochim. Biophys. Acta, 160, 145-155; Strickland & Mercola, 1976, Biochemistry, 15, 3875-3884). The free energy of unfolding of insulin was previously calculated from such studies to be 4.5 kcal/mol (Kaarsholm, N. C., et al, 1993, Biochemistry, 32, 10773-8).

Insulin CD spectrum in the near UV range (330-250-nm) reflects the environment of the tyrosine chromophore with contributions from the disulfide bonds. Since tyrosine residues are part of the insulin's dimer surface, changes in molar ellipticity at this region (especially at 276 nm) reflect on insulin's association state. Another way to measure insulin's association state is by application of size-exclusion chromatography under non-dissociating conditions as known in the art and described in the examples.

The following is a non-limiting list of aspects according to the invention:
1. An N-terminally modified insulin consisting of a peptide part, N-terminal modification groups and an albumin binding moiety, wherein the peptide part has at least one disulphide bond which is not present in human insulin.
2. An N-terminally modified insulin consisting of a peptide part, N-terminal modification groups and an albumin binding moiety, wherein the peptide part has two or more cysteine substitutions and the three disulfide bonds of human insulin are retained.
3. An N-terminally modified insulin according to any one of the preceding aspects to the extent possible, wherein the sites of cysteine substitutions are chosen in such a way that the introduced cysteine residues are placed in the three dimensional structure of the folded N-terminally modified insulin to allow for the formation of one or more additional disulfide bonds not present in human insulin, and wherein the N-terminally modified insulin has at least 5% of the insulin receptor affinity of an insulin peptide having the same peptide part, the same N-terminal modification groups and the same albumin binding moiety but without any disulphide bonds which are not present in human insulin
4. An N-terminally modified insulin according to any one of the preceding aspects to the extent possible, wherein the insulin receptor affinity is at least 10%, such as at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100% of an insulin peptide having the same peptide part, the same N-terminal modification groups and the same albumin binding moiety but without any disulphide bonds which are not present in human insulin
5. An N-terminally modified insulin according to any one of the preceding aspects to the extent possible, wherein the modification groups are one or two organic substituents which are each having a molecular weight (MW) below 200 g per mol conjugated to the N-terminals of the parent insulin.
6. An N-terminally modified insulin according to any one of the preceding aspects to the extent possible, wherein modification groups are designated Y′ and Z in Chem. V (illustrated for the A-chain N-terminal):

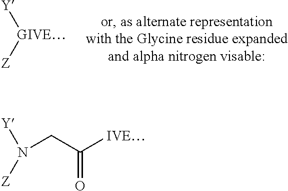

and wherein Y′ and Z are attached to the N-terminal amino acids of the insulin peptide.
7. An N-terminally modified insulin according to any one of the preceding aspects to the extent possible, wherein Y′ and Z are different and
Y′ is R—C(═X)—,
Z is H,
R is H, NH₂, straight chain or branched C1-C4 alkyl, straight chain or branched C2-C4 alkyl substituted with dimethylamino, diethylamino, dipropylamino, dimethylammonium, diethylammonium, or dipropylammonium, CO₂H, or —OCH₂CO₂H, C5-C6 cycloalkyl, substituted C5-C6 cycloalkyl, 5- or 6 membered saturated heterocyclyl, substituted 5- or 6 membered saturated heterocyclyl, and
X is O or S.
   8. An N-terminally modified insulin according to any one of the preceding aspects to the extent possible, wherein Y′ and Z are different and
Y′ is R—C(═X)—,
Z is H,
R is H, NH₂, straight chain or branched C1-C4 alkyl, C1-C4 alkyl substituted with CO₂H, or —OCH₂CO₂H, C5-C6 cycloalkyl, 5- or 6 membered saturated heterocyclyl, and
   X is O or S.
      9. An N-terminally modified insulin according to any one of the preceding aspects to the extent possible, wherein Y′ and Z are different and
Y′ is R—C(═X)—,
Z is H,
R is H, NH₂, straight chain or branched C1-C4 alkyl, C1-C4 alkyl substituted with CO₂H, or —OCH₂CO₂H, and
   X is O.
      10. An N-terminally modified insulin according to any one of the preceding aspects to the extent possible, wherein Y′ and Z are different and
Y′ is straight chain or branched C1-C4 alkyl, straight chain or branched C2-C4 acyl substituted with dimethylamino, diethylamino, dipropylamino, trimethylammonium, triethylammonium or dipropylammonium, 5- or 6 membered saturated heterocyclyl, substituted 5- or 6 membered saturated heterocyclyl, amidinyl, and
Z is H.
   11. An N-terminally modified insulin according to any one of the preceding aspects to the extent possible, wherein Y′ and Z are different and
Y′ is straight chain C1-C4 alkyl, 5- or 6 membered saturated heterocyclyl, and
Z is H.
   12. An N-terminally modified insulin according to any one of the preceding aspects to the extent possible, wherein Y′=Z=C1-C4 alkyl.
   13. An N-terminally modified insulin according to any one of the preceding aspects to the extent possible, wherein Y′ and Z are the same and selected from the group consisting of: methyl, ethyl, n-propyl, sec-propyl, n-butyl, and di-i-butyl.
   14. An N-terminally modified insulin according to any one of the preceding aspects to the extent possible, wherein Y′ and Z are the same and selected from dimethyl and diethyl
   15. An N-terminally modified insulin according to any one of the preceding aspects to the extent possible, wherein Y′ and Z are the same and methyl.
   16. An N-terminally modified insulin according to any one of the preceding aspects to the extent possible, wherein the N-terminal modification is positively charged at physiological pH.
   17. An N-terminally modified insulin according to any one of the preceding aspects to the extent possible, wherein the N-terminal modification is selected from the group consisting of: N,N-di-C1-4alkyl, N-amidinyl, 4-(N,N-dimethylamino)butanoyl, 3-(1-piperidinyl)propionyl, 3-(N,N-dimethylamino)propionyl, N,N-dimethyl-glycyl.
   18. An N-terminally modified insulin according to any one of the preceding aspects to the extent possible, wherein the N-terminal modification is N,N-di-C1-4alkyl.
   19. An N-terminally modified insulin according to any one of the preceding aspects to the extent possible, wherein the N-terminal modification is N,N-dimethyl or N,N-diethyl.
   20. An N-terminally modified insulin according to any one of the preceding aspects to the extent possible, wherein the N-terminal modification group is not malonyl or succinyl.
   21. An N-terminally modified insulin according to any one of the preceding aspects to the extent possible, wherein the N-terminal modification group is not malonyl.
   22. An N-terminally modified insulin according to any one of the preceding aspects to the extent possible, wherein the N-terminal modification group is not succinyl.
   23. An N-terminally modified insulin according to any one of the preceding aspects to the extent possible, wherein the N-terminal modification group is selected from the group consisting of: N,N-dimethyl, N,N-diethyl, carbamoyl, formyl, acetyl, propionyl, butyryl, glutaryl, and diglycolyl.
   24. An N-terminally modified insulin according to any one of the preceding aspects to the extent possible, wherein the N-terminal modification is neutral at physiological pH.
   25. An N-terminally modified insulin according to any one of the preceding aspects to the extent possible, wherein the N-terminal modification is selected from the group consisting of: Carbamoyl, thiocarbamoyl, formyl, acetyl, propionyl, butyryl, and pyroglutamyl.
   26. An N-terminally modified insulin according to any one of the preceding aspects to the extent possible, wherein the N-terminal modification is selected from the group consisting of: Carbamoyl, acetyl, propionyl, and butyryl.
   27. An N-terminally modified insulin according to any one of the preceding aspects to the extent possible, wherein the N-terminal modification is carbamoyl.
   28. An N-terminally modified insulin according to any one of the preceding aspects to the extent possible, wherein the N-terminal modification is acetyl.
   29. An N-terminally modified insulin according to any one of the preceding aspects to the extent possible, wherein the N-terminal modification is negatively charged at physiological pH.
   30. An N-terminally modified insulin according to any one of the preceding aspects to the extent possible, wherein the N-terminal modification is selected from the group consisting of: oxalyl, glutaryl and diglycolyl.
   31. An N-terminally modified insulin according to any one of the preceding aspects to the extent possible, wherein the N-terminal modification is selected from the group consisting of: oxalyl, glutaryl and diglycolyl.
   32. An N-terminally modified insulin according to any one of the preceding aspects to the extent possible, wherein the N-terminal modification is glutaryl.
   33. An N-terminally modified insulin according to any one of the preceding aspects to the extent possible, wherein the N-terminal modification is diglycolyl.
   34. An N-terminally modified insulin according to any one of the preceding aspects to the extent possible, wherein the peptide part is human insulin substituted with at least two cysteines in positions which are selected from the group consisting of:

A10C, B1C;

A10C, B2C;

A10C, B3C;

A10C, B4C;

A10C, B5C; and

B1C, B4C.

35. An N-terminally modified insulin according to any one of the preceding aspects to the extent possible, wherein the positions for cysteine substitution are selected from the group consisting of:

A10C, B1C;

A10C, B2C;

A10C, B3C;

A10C, B4C; and

B1C, B4C.

36. An N-terminally modified insulin according to any one of the preceding aspects to the extent possible, wherein the positions for cysteine substitution are selected from the group consisting of:

A10C, B1C;

A10C, B2C;

A10C, B3C; and

A10C, B4C.

37. An N-terminally modified insulin according to any one of the preceding aspects to the extent possible, wherein the positions for cysteine substitution are selected from the group consisting of:

A10C, B3C; and

A10C, B4C.

38. An N-terminally modified insulin according to any one of the preceding aspects to the extent possible, wherein the positions for cysteine substitution are A100 and B3C.
39. An N-terminally modified insulin according to any one of the preceding aspects to the extent possible, which further comprises mutations such that at least one hydrophobic amino acid has been substituted with a hydrophilic amino acid, and wherein said substitution is within or in close proximity to one or more protease cleavage sites of the insulin.
40. An N-terminally modified insulin according to any one of the preceding aspects to the extent possible, wherein the peptide part in addition to two or more cysteine substitutions comprises one or more substituted amino acids selected from the group consisting of: A8H, A14E, A14H, A21G, B1G, B3Q, B3E, B3T, B3V, B3L, B16H, B16E, B25A, B25H, B25N, B27E, B27P, B28E, desB1, desB27 and desB30.
41. An N-terminally modified insulin according to any one of the preceding aspects to the extent possible, wherein the peptide part in addition to two or more cysteine substitutions comprises one or more substituted amino acids selected from the group consisting of: A14E, A14H, A21G, desB1, B1G, B3Q, B3E, B16H, B16E, B25H, desB27, and desB30.
42. An N-terminally modified insulin according to any one of the preceding aspects to the extent possible, wherein the peptide part in addition to two or more cysteine substitutions comprises one or more substituted amino acids selected from the group consisting of: A14E, desB1, B1G, B16H, B16E, B25H, desB27, and desB30.
43. An N-terminally modified insulin according to any one of the preceding aspects to the extent possible, wherein the peptide part in addition to two or more cysteine substitutions comprises one or more substituted amino acids selected from the group consisting of: A14E, B16H, B25H, desB27, and desB30.
44. An N-terminally modified insulin according to any one of the preceding aspects to the extent possible, wherein the peptide part is selected from the group consisting of: A10C, A14E, B1C, B16H, B25H, desB30 human insulin; A10C, A14E, B1C, B25H, desB30 human insulin; A10C, A14E, B2C, B16H, B25H, desB30 human insulin; A10C, A14E, B2C, B25H, desB30 human insulin; A10C, A14E, B3C, B16H, B25H, desB30 human insulin; A10C, A14E, B3C, B25H, desB27, desB30 human insulin; A10C, A14E, B3C, B25H, desB30 human insulin; A10C, A14E, B3C, desB27, desB30 human insulin; A10C, A14E, B3C, B16H, B25H, desB27, desB30 human insulin; A10C, A14E, B16H, desB27, desB30 human insulin; A10C, A14E, B4C, B16H, B25H, desB30 human insulin; A10C, A14E, B4C, B25A, desB30 human insulin; A10C, A14E, B4C, B25H, B28E, desB30 human insulin; A10C, A14E, B4C, B25H, desB27, desB30 human insulin; A10C, A14E, B4C, B25H, desB30 human insulin; A10C, A14E, B4C, B25N, B27E, desB30 human insulin; A10C, A14E, B4C, B25N, desB27, desB30 human insulin; A10C, A14E, desB1, B4C, B25H, desB30 human insulin; A10C, A14H, B4C, B25H, desB30 human insulin; A10C, A14E, B1C, B16H, B25H, desB30 human insulin; A10C, A14E, B2C, B16H, B25H, desB30 human insulin; and A10C, A14E, B4C, B16H, B25H, desB30 human insulin.
45. An N-terminally modified insulin according to any one of the preceding aspects to the extent possible, wherein the peptide part is selected from the group consisting of: A10C, A14E, B3C, B16H, B25H, desB30 human insulin; A10C, A14E, B3C, B25H, desB27, desB30 human insulin; A10C, A14E, B3C, B25H, desB30 human insulin; A10C, A14E, B3C, desB27, desB30 human insulin; A10C, A14E, B3C, B16H, B25H, desB27, desB30 human insulin; A10C, A14E, B16H, desB27, desB30 human insulin.
46. An N-terminally modified insulin according to any one of the preceding aspects to the extent possible, wherein the albumin binding moiety is a side chain consisting of a fatty acid or a fatty diacid attached to the insulin, optionally via a linker, to an amino acid position of the peptide part.
47. An N-terminally modified insulin according to any one of the preceding aspects to the extent possible, wherein the peptide part comprises only one lysine residue and the albumin binding moiety is attached, optionally via a linker, to said lysine residue.
48. An N-terminally modified insulin according to any one of the preceding aspects to the extent possible, wherein the albumin binding moiety has the general formula Acy-AA1ₙ-AA2ₘ-AA3ₚ- (Chem. IV),
wherein

n is 0 or an integer in the range from 1 to 3;

m is 0 or an integer in the range from 1 to 10;

p is 0 or an integer in the range from 1 to 10;

Acy is a fatty acid or a fatty diacid comprising from about 14 to about 20 carbon atoms;

AA1 is a neutral linear or cyclic amino acid residue;

AA2 is an acidic amino acid residue;

AA3 is a neutral, alkyleneglycol-containing amino acid residue;

the order by which AA1, AA2 and AA3 appears in the formula can be interchanged independently; AA2 can occur several times along the formula (e.g., Acy-AA2-AA3₂-AA2-); AA2 can occur independently (=being different) several times along the formula (e.g., Acy-AA2-AA3₂-AA2-); the connections between Acy, AA1, AA2 and/or AA3 are amide (peptide) bonds which, formally, can be obtained by removal of a hydrogen atom or a hydroxyl group (water) from each of Acy, AA1, AA2 and AA3; and attachment to the peptide part can be from the C-terminal end of a AA1, AA2, or AA3 residue in the acyl moiety of the Chem. IV or from one of the side chain(s) of an AA2 residue present in the moiety of Chem. IV.
49. An N-terminally modified insulin according to any one of the preceding aspects to the extent possible, which is selected from the group consisting of:

A1(N^{α},N^{α}-Dimethyl), A10C, A14E, B1(N^{α},N^{α}-dimethyl), B3C, B25H, desB27, B29K(N^{ε}octadecanedioyl-gGlu), desB30 human insulin

A1(N^{α},N^{α}-Dimethyl), A10C, A14E, B1(N^{α},N^{α}-dimethyl), B3C, B25H, B29K(N^{ε}eicosanedioyl-gGlu-2×OEG), desB30 human insulin

A1(N^{α},N^{α}-Dimethyl), A10C, A14E, B1(N^{α},N^{α}-dimethyl), B3C, B25H, B29K(N^{ε}octadecanedioyl-gGlu), desB30 human insulin

A1(N^{α},N^{α}-Dimethyl), A10C, A14E, B1(N^{α},N^{α}-dimethyl), B3C, B16H, B25H, B29K(N^{ε}octadecanedioyl-gGlu), desB30 human insulin

A1(N^{α},N^{α}-Dimethyl), A10C, A14E, B1(N^{α},N^{α}-dimethyl), B3C, desB27, B29K(N^{ε}octadecanedioyl-gGlu), desB30 human insulin

A1(N^{α},N^{α}-Dimethyl), A10C, A14E, B1G(N^{α},N^{α}-dimethyl), B3C, B25H, B29K(N^{ε}octadecanedioyl-gGlu), desB30 human insulin

A1(N^{α},N^{α}-Dimethyl), A10C, A14E, B1G(N^{α},N^{α}-dimethyl), B3C, B25H, desB27, B29K(N^{ε}octadecanedioyl-gGlu), desB30 human insulin

A1(N^{α},N^{α}-Dimethyl), A10C, A14E, B1G(N^{α},N^{α}-dimethyl), B3C, B16H, B25H, B29K(N^{ε}octadecanedioyl-gGlu), desB30 human insulin

A1(N^{α},N^{α}-Dimethyl), A10C, A14E, B1G(N^{α},N^{α}-dimethyl), B3C, desB27, B29K(N^{ε}octadecanedioyl-gGlu), desB30 human insulin

A1(N^{α},N^{α}-Dimethyl), A10C, A14E, B1(N^{α},N^{α}-dimethyl), B3C, desB27, B29K(N^{ε}octadecanedioyl-gGlu-2×OEG), desB30 human insulin

A1(N^{α},N^{α}-Dimethyl), A10C, A14E, B1(N^{α},N^{α}-dimethyl), B3C, B25H, desB27, B29K(N^{ε}octadecanedioyl-gGlu-2×OEG), desB30 human insulin

A1(N^{α},N^{α}-Dimethyl), A10C, A14E, B1(N^{α},N^{α}-dimethyl), B3C, B25H, B29K(N^{ε}octadecanedioyl-gGlu-2×OEG), desB30 human insulin

A1(N^{α},N^{α}-Dimethyl), A10C, A14E, B1(N^{α},N^{α}-dimethyl), B3C, B16H, B25H, B29K(N^{ε}octadecanedioyl-gGlu-2×OEG), desB30 human insulin

A1(N^{α},N^{α}-Dimethyl), A10C, A14E, B1G(N^{α},N^{α}-dimethyl), B3C, desB27, B29K(N^{ε}octadecanedioyl-gGlu-2×OEG), desB30 human insulin

A1(N^{α},N^{α}-Dimethyl), A10C, A14E, B1G(N^{α},N^{α}-dimethyl), B3C, B25H, desB27, B29K(N^{ε}octadecanedioyl-gGlu-2×OEG), desB30 human insulin

A1(N^{α},N^{α}-Dimethyl), A10C, A14E, B1G(N^{α},N^{α}-dimethyl), B3C, B25H, B29K(N^{ε}octadecanedioyl-gGlu-2×OEG), desB30 human insulin

A1(N^{α},N^{α}-Dimethyl), A10C, A14E, B1G(N^{α},N^{α}-dimethyl), B3C, B16H, B25H, B29K(N^{ε}octadecanedioyl-gGlu-2×OEG), desB30 human insulin

A1(N^{α},N^{α}-Dimethyl), A10C, A14E, B1(N^{α},N^{α}-dimethyl), B3C, B25H, desB27, B29K(N^{ε}eicosanedioyl-gGlu), desB30 human insulin

A1(N^{α},N^{α}-Dimethyl), A10C, A14E, B1(N^{α},N^{α}-dimethyl), B3C, B25H, B29K(N^{ε}eicosanedioyl-gGlu), desB30 human insulin

A1(N^{α},N^{α}-Dimethyl), A10C, A14E, B1(N^{α},N^{α}-dimethyl), B3C, B16H, B25H, B29K(N^{ε}eicosanedioyl-gGlu), desB30 human insulin

A1(N^{α},N^{α}-Dimethyl), A10C, A14E, B1(N^{α},N^{α}-dimethyl), B3C, desB27, B29K(N^{ε}eicosanedioyl-gGlu), desB30 human insulin

A1(N^{α},N^{α}-Dimethyl), A10C, A14E, B1G(N^{α},N^{α}-dimethyl), B3C, B25H, B29K(N^{ε}eicosanedioyl-gGlu), desB30 human insulin

A1(N^{α},N^{α}-Dimethyl), A10C, A14E, B1G(N^{α},N^{α}-dimethyl), B3C, B25H, desB27, B29K(N^{ε}eicosanedioyl-gGlu), desB30 human insulin

A1(N^{α},N^{α}-Dimethyl), A10C, A14E, B1G(N^{α},N^{α}-dimethyl), B3C, B16H, B25H, B29K(N^{ε}eicosanedioyl-gGlu), desB30 human insulin

A1(N^{α},N^{α}-Dimethyl), A10C, A14E, B1G(N^{α},N^{α}-dimethyl), B3C, desB27, B29K(N^{ε}eicosanedioyl-gGlu), desB30 human insulin

A1(N^{α},N^{α}-Dimethyl), A10C, A14E, B1(N^{α},N^{α}-dimethyl), B3C, desB27, B29K(N^{ε}eicosanedioyl-gGlu-2×OEG), desB30 human insulin

A1(N^{α},N^{α}-Dimethyl), A10C, A14E, B1(N^{α},N^{α}-dimethyl), B3C, B25H, desB27, B29K(N^{ε}eicosanedioyl-gGlu-2×OEG), desB30 human insulin

A1(N^{α},N^{α}-Dimethyl), A10C, A14E, B1(N^{α},N^{α}-dimethyl), B3C, B16H, B25H, B29K(N^{ε}eicosanedioyl-gGlu-2×OEG), desB30 human insulin

A1(N^{α},N^{α}-Dimethyl), A10C, A14E, B1G(N^{α},N^{α}-dimethyl), B3C, desB27, B29K(N^{ε}eicosanedioyl-gGlu-2×OEG), desB30 human insulin

A1(N^{α},N^{α}-Dimethyl), A10C, A14E, B1G(N^{α},N^{α}-dimethyl), B3C, B25H, desB27, B29K(N^{ε}eicosanedioyl-gGlu-2×OEG), desB30 human insulin

A1(N^{α},N^{α}-Dimethyl), A10C, A14E, B1G(N^{α},N^{α}-dimethyl), B3C, B25H, B29K(N^{ε}eicosanedioyl-gGlu-2×OEG), desB30 human insulin

A1(N^{α},N^{α}-Dimethyl), A10C, A14E, B1G(N^{α},N^{α}-dimethyl), B3C, B16H, B25H, B29K(N^{ε}eicosanedioyl-gGlu-2×OEG), desB30 human insulin

A1(N^{α}-Carbamoyl), A10C, A14E, B1(N^{α}-carbamoyl), B3C, desB27, B29K(N^{ε}octadecanedioyl-gGlu-2×OEG), desB30 human insulin

A1(N^{α}Carbamoyl), A10C, A14E, B1(N^{α}carbamoyl), B3C, B25H, desB27, B29K(N^{ε}octadecanedioyl-gGlu), desB30 human insulin

A1(N^{α}-Carbamoyl), A10C, A14E, B1(N^{α}-carbamoyl), B4C, B25H, B29K(N^{ε}eicosanedioyl-gGlu-2×OEG), desB30 human insulin

A1(N^{α}-Carbamoyl), A10C, A14E, B1(N^{α}-carbamoyl), B3C, desB27, B29K(N^{ε}octadecanedioyl-gGlu), desB30 human insulin

A1(N^{α}Carbamoyl), A10C, A14E, B1(N^{α}carbamoyl), B3C, B25H, B29K(N^{ε}octadecanedioyl-gGlu), desB30 human insulin

A1(N^{α},N^{α}-Carbamoyl), A10C, A14E, B1(N^{α},N^{α}-carbamoyl), B3C, B16H, B25H, B29K(N^{ε}octadecanedioyl-gGlu), desB30 human insulin

A1(N^{α}-Carbamoyl), A10C, A14E, B1(N^{α}-carbamoyl), B3C, B25H, desB27, B29K(N^{ε}octadecanedioyl-gGlu-2×OEG), desB30 human insulin

A1(N^{α}-Carbamoyl), A10C, A14E, B1(N^{α}-carbamoyl), B3C, B25H, B29K(N^{ε}octadecanedioyl-gGlu-2×OEG), desB30 human insulin

A1(N^{α}-Carbamoyl), A10C, A14E, B1(N^{α}-carbamoyl), B3C, B16H, B25H, B29K(N^{ε}octadecanedioyl-gGlu-2×OEG), desB30 human insulin

A1(N^{α}-Carbamoyl), A10C, A14E, B1(N^{α}-carbamoyl), B3C, B25H, desB27, B29K(N^{ε}eicosanedioyl-gGlu), desB30 human insulin

A1(N^{α}-Carbamoyl), A10C, A14E, B1(N^{α}-carbamoyl), B3C, desB27, B29K(N^{ε}eicosanedioyl-gGlu), desB30 human insulin

A1(N^{α}-Carbamoyl), A10C, A14E, B1(N^{α}-carbamoyl), B3C, B25H, B29K(N^{ε}eicosanedioyl-gGlu), desB30 human insulin

A1(N^{α}-Carbamoyl), A10C, A14E, B1(N^{α}-carbamoyl), B3C, B16H, B25H, B29K(N^{ε}eicosanedioyl-gGlu), desB30 human insulin

A1(N^{α}-Carbamoyl), A10C, A14E, B1(N^{α}-carbamoyl), B3C, B25H, desB27, B29K(N^{ε}eicosanedioyl-gGlu-2×OEG), desB30 human insulin

A1(N^{α}-Carbamoyl), A10C, A14E, B1(N^{α}-carbamoyl), B3C, desB27, B29K(N^{ε}eicosanedioyl-gGlu-2×OEG), desB30 human insulin

A1(N^{α}-Carbamoyl), A10C, A14E, B1(N^{α}-carbamoyl), B3C, B25H, B29K(N^{ε}eicosanedioyl-gGlu-2×OEG), desB30 human insulin

A1(N^{α}-Carbamoyl), A10C, A14E, B1(N^{α}-carbamoyl), B3C, B16H, B25H, B29K(N^{ε}eicosanedioyl-gGlu-2×OEG), desB30 human insulin

A1(N^{α}-Acetyl), A10C, A14E, B1(N^{α}-acetyl), B3C, B25H, desB27, B29K(N^{ε}octadecanedioyl-gGlu), desB30 human insulin

A1(N^{α}-Acetyl), A10C, A14E, B1(N^{α}-acetyl), B3C, desB27, B29K(N^{ε}octadecanedioyl-gGlu-2×OEG), desB30 human insulin

A1(N^{α}-Acetyl), A10C, A14E, B1(N^{α}-acetyl), B3C, desB27, B29K(N^{ε}octadecanedioyl-gGlu), desB30 human insulin

A1(N^{α}-Acetyl), A10C, A14E, B1(N^{α}-acetyl), B3C, B25H, B29K(N^{ε}octadecanedioyl-gGlu), desB30 human insulin

A1(N^{α}-Acetyl), A10C, A14E, B1(N^{α}-acetyl), B3C, B16H, B25H, B29K(N^{ε}octadecanedioyl-gGlu), desB30 human insulin

A1(N^{α}-Acetyl), A10C, A14E, B1(N^{α}-acetyl), B3C, B25H, desB27, B29K(N^{ε}octadecanedioyl-gGlu-2×OEG), desB30 human insulin

A1(N^{α}-Acetyl), A10C, A14E, B1(N^{α}-acetyl), B3C, B25H, B29K(N^{ε}octadecanedioyl-gGlu-2×OEG), desB30 human insulin

A1(N^{α}-Acetyl), A10C, A14E, B1(N^{α}-acetyl), B3C, B16H, B25H, B29K(N^{ε}octadecanedioyl-gGlu-2×OEG), desB30 human insulin

A1(N^{α}-Acetyl), A10C, A14E, B1(N^{α}-acetyl), B3C, B25H, desB27, B29K(N^{ε}eicosanedioyl-gGlu), desB30 human insulin

A1(N^{α}-Acetyl), A10C, A14E, B1(N^{α}-acetyl), B3C, desB27, B29K(N^{ε}eicosanedioyl-gGlu), desB30 human insulin

A1(N^{α}-Acetyl), A10C, A14E, B1(N^{α}-acetyl), B3C, B25H, B29K(N^{ε}eicosanedioyl-gGlu), desB30 human insulin

A1(N^{α}-Acetyl), A10C, A14E, B1(N^{α}-acetyl), B3C, B16H, B25H, B29K(N^{ε}eicosanedioyl-gGlu), desB30 human insulin

A1(N^{α}-Acetyl), A10C, A14E, B1(N^{α}-acetyl), B3C, B25H, desB27, B29K(N^{ε}eicosanedioyl-gGlu-2×OEG), desB30 human insulin

A1(N^{α}-Acetyl), A10C, A14E, B1(N^{α}-acetyl), B3C, desB27, B29K(N^{ε}eicosanedioyl-gGlu-2×OEG), desB30 human insulin

A1(N^{α}-Acetyl), A10C, A14E, B1(N^{α}-acetyl), B3C, B25H, B29K(N^{ε}eicosanedioyl-gGlu-2×OEG), desB30 human insulin

A1(N^{α}-Acetyl), A10C, A14E, B1(N^{α}-acetyl), B3C, B16H, B25H, B29K(N^{ε}eicosanedioyl-gGlu-2×OEG), desB30 human insulin

A1(N^{α}-Glutaryl), A10C, A14E, B1(N^{α}-glutaryl), B3C, B25H, desB27, B29K(N^{ε}octadecanedioyl-gGlu), desB30 human insulin

A1(N^{α}-Glutaryl), A10C, A14E, B1(N^{α}-glutaryl), B3C, desB27, B29K(N^{ε}octadecanedioyl-gGlu-2×OEG), desB30 human insulin

A1(N^{α}-Glutaryl), A10C, A14E, B1(N^{α}-glutaryl), B3C, desB27, B29K(N^{ε}octadecanedioyl-gGlu), desB30 human insulin

A1(N^{α}-Glutaryl), A10C, A14E, B1(N^{α}-glutaryl), B3C, B25H, B29K(N^{ε}octadecanedioyl-gGlu), desB30 human insulin

A1(N^{α}-Glutaryl), A10C, A14E, B1(N^{α}-glutaryl), B3C, B16H, B25H, B29K(N^{ε}octadecanedioyl-gGlu), desB30 human insulin

A1(N^{α}-Glutaryl), A10C, A14E, B1(N^{α}-glutaryl), B3C, B25H, desB27, B29K(N^{ε}octadecanedioyl-gGlu-2×OEG), desB30 human insulin

A1(N^{α}-Glutaryl), A10C, A14E, B1(N^{α}-glutaryl), B3C, B25H, B29K(N^{ε}octadecanedioyl-gGlu-2×OEG), desB30 human insulin

A1(N^{α}-Glutaryl), A10C, A14E, B1(N^{α}-glutaryl), B3C, B16H, B25H, B29K(N^{ε}octadecanedioyl-gGlu-2×OEG), desB30 human insulin

A1(N^{α}-Glutaryl), A10C, A14E, B1(N^{α}-glutaryl), B3C, B25H, desB27, B29K(N^{ε}eicosanedioyl-gGlu), desB30 human insulin

A1(N^{α}-Glutaryl), A10C, A14E, B1(N^{α}-glutaryl), B3C, desB27, B29K(N^{ε}eicosanedioyl-gGlu), desB30 human insulin

A1(N^{α}-Glutaryl), A10C, A14E, B1(N^{α}-glutaryl), B3C, B25H, B29K(N^{ε}eicosanedioyl-gGlu), desB30 human insulin

A1(N^{α}-Glutaryl), A10C, A14E, B1(N^{α}-glutaryl), B3C, B16H, B25H, B29K(N^{ε}eicosanedioyl-gGlu), desB30 human insulin

A1(N^{α}-Glutaryl), A10C, A14E, B1(N^{α}-glutaryl), B3C, B25H, desB27, B29K(N^{ε}eicosanedioyl-gGlu-2×OEG), desB30 human insulin

A1(N^{α}-Glutaryl), A10C, A14E, B1(N^{α}-glutaryl), B3C, desB27, B29K(N^{ε}eicosanedioyl-gGlu-2×OEG), desB30 human insulin

A1(N^{α}-Glutaryl), A10C, A14E, B1(N^{α}-glutaryl), B3C, B25H, B29K(N^{ε}eicosanedioyl-gGlu-2×OEG), desB30 human insulin

A1(N^{α}-Glutaryl), A10C, A14E, B1(N^{α}-glutaryl), B3C, B16H, B25H, B29K(N^{ε}eicosanedioyl-gGlu-2×OEG), desB30 human insulin

A1(N^{α}-Diglycolyl), A10C, A14E, B1(N^{α}-diglycolyl), B3C, B25H, desB27, B29K(N^{ε}octadecanedioyl-gGlu), desB30 human insulin

A1(N^{α}-Diglycolyl), A10C, A14E, B1(N^{α}-diglycolyl), B3C, desB27, B29K(N^{ε}octadecanedioyl-gGlu-2×OEG), desB30 human insulin

A1(N^{α}-Diglycolyl), A10C, A14E, B1(N^{α}-diglycolyl), B3C, desB27, B29K(N^{ε}octadecanedioyl-gGlu), desB30 human insulin

A1(N^{α}-Diglycolyl), A10C, A14E, B1(N^{α}-diglycolyl), B3C, B25H, B29K(N^{ε}octadecanedioyl-gGlu), desB30 human insulin

A1(N^{α}-Diglycolyl), A10C, A14E, B1(N^{α}-diglycolyl), B3C, B16H, B25H, B29K(N^{ε}octadecanedioyl-gGlu), desB30 human insulin

A1(N^{α}-Diglycolyl), A10C, A14E, B1(N^{α}-diglycolyl), B3C, B25H, desB27, B29K(N^{ε}octadecanedioyl-gGlu-2×OEG), desB30 human insulin

A1(N^{α}-Diglycolyl), A10C, A14E, B1(N^{α}-diglycolyl), B3C, B25H, B29K(N^{ε}octadecanedioyl-gGlu-2×OEG), desB30 human insulin

A1(N^{α}-Diglycolyl), A10C, A14E, B1(N^{α}-diglycolyl), B3C, B16H, B25H, B29K(N^{ε}octadecanedioyl-gGlu-2×OEG), desB30 human insulin

A1(N^{α}-Diglycolyl), A10C, A14E, B1(N^{α}-diglycolyl), B3C, B25H, desB27, B29K(N^{ε}eicosanedioyl-gGlu), desB30 human insulin

A1(N^{α}-Diglycolyl), A10C, A14E, B1(N^{α}-diglycolyl), B3C, desB27, B29K(N^{ε}eicosanedioyl-gGlu), desB30 human insulin

A1(N^{α}-Diglycolyl), A10C, A14E, B1(N^{α}-diglycolyl), B3C, B25H, B29K(N^{ε}eicosanedioyl-gGlu), desB30 human insulin

A1(N^{α}-Diglycolyl), A10C, A14E, B1(N^{α}-diglycolyl), B3C, B16H, B25H, B29K(N^{ε}eicosanedioyl-gGlu), desB30 human insulin

A1(N^{α}-Diglycolyl), A10C, A14E, B1(N^{α}-diglycolyl), B3C, B25H, desB27, B29K(N^{ε}eicosanedioyl-gGlu-2×OEG), desB30 human insulin

A1(N^{α}-Diglycolyl), A10C, A14E, B1(N^{α}-diglycolyl), B3C, desB27, B29K(N^{ε}eicosanedioyl-gGlu-2×OEG), desB30 human insulin

A1(N^{α}-Diglycolyl), A10C, A14E, B1(N^{α}-diglycolyl), B3C, B25H, B29K(N^{ε}eicosanedioyl-gGlu-2×OEG), desB30 human insulin

A1(N^{α}-Diglycolyl), A10C, A14E, B1(N^{α}-diglycolyl), B3C, B16H, B25H, B29K(N^{ε}eicosanedioyl-gGlu-2×OEG), desB30 human insulin

50. An N-terminally modified insulin according to any one of the preceding aspects to the extent possible, which is selected from the group consisting of:

A1(N^{α}-Carbamoyl), A10C, A14E, B1(N^{α}-carbamoyl), B3C, desB27, B29K(N^{ε}octadecanedioyl-gGlu-2×OEG), desB30 human insulin

A1(N^{α}-Carbamoyl), A10C, A14E, B1(N^{α}-carbamoyl), B3C, desB27, B29K(N^{ε}octadecanedioyl-gGlu), desB30 human insulin

A1(N^{α}-Carbamoyl), A10C, A14E, B1(N^{α}-carbamoyl), B3C, desB27, B29K(N^{ε}eicosanedioyl-gGlu), desB30 human insulin

A1(N^{α}-Carbamoyl), A10C, A14E, B1(N^{α}-carbamoyl), B3C, desB27, B29K(N^{ε}eicosanedioyl-gGlu-2×OEG), desB30 human insulin

A1(N^{α}-Acetyl), A10C, A14E, B1(N^{α}-acetyl), B3C, desB27, B29K(N^{ε}octadecanedioyl-gGlu-2×OEG), desB30 human insulin

A1(N^{α}-Acetyl), A10C, A14E, B1(N^{α}-acetyl), B3C, desB27, B29K(N^{ε}octadecanedioyl-gGlu), desB30 human insulin

A1(N^{α}-Acetyl), A10C, A14E, B1(N^{α}-acetyl), B3C, desB27, B29K(N^{ε}eicosanedioyl-gGlu-2×OEG), desB30 human insulin

A1(N^{α}-Glutaryl), A10C, A14E, B1(N^{α}-glutaryl), B3C, desB27, B29K(N^{ε}octadecanedioyl-gGlu-2×OEG), desB30 human insulin

A1(N^{α}-Glutaryl), A10C, A14E, B1(N^{α}-glutaryl), B3C, desB27, B29K(N^{ε}octadecanedioyl-gGlu), desB30 human insulin

A1(N^{α}-Glutaryl), A10C, A14E, B1(N^{α}-glutaryl), B3C, desB27, B29K(N^{ε}eicosanedioyl-gGlu), desB30 human insulin

A1(N^{α}-Glutaryl), A10C, A14E, B1(N^{α}-glutaryl), B3C, desB27, B29K(N^{ε}eicosanedioyl-gGlu-2×OEG), desB30 human insulin

A1(N^{α}-Diglycolyl), A10C, A14E, B1(N^{α}-diglycolyl), B3C, desB27, B29K(N^{ε}octadecanedioyl-gGlu-2×OEG), desB30 human insulin

A1(N^{α}-Diglycolyl), A10C, A14E, B1(N^{α}-diglycolyl), B3C, desB27, B29K(N^{ε}octadecanedioyl-gGlu), desB30 human insulin

A1(N^{α}-Diglycolyl), A10C, A14E, B1(N^{α}-diglycolyl), B3C, desB27, B29K(N^{ε}eicosanedioyl-gGlu), desB30 human insulin

A1(N^{α}-Diglycolyl), A10C, A14E, B1(N^{α}-diglycolyl), B3C, desB27, B29K(N^{ε}eicosanedioyl-gGlu-2×OEG), desB30 human insulin

51. An N-terminally modified insulin according to any one of the preceding aspects to the extent possible, which is selected from the group consisting of:

A1(N^{α}-Carbamoyl), A10C, A14E, B1(N^{α}-carbamoyl), B3C, desB27, B29K(N^{ε}octadecanedioyl-gGlu-2×OEG), desB30 human insulin

A1(N^{α}-Carbamoyl), A10C, A14E, B1(N^{α}-carbamoyl), B3C, desB27, B29K(N^{ε}octadecanedioyl-gGlu), desB30 human insulin

A1(N^{α}-Carbamoyl), A10C, A14E, B1(N^{α}-carbamoyl), B3C, desB27, B29K(N^{ε}eicosanedioyl-gGlu), desB30 human insulin

A1(N^{α}-Carbamoyl), A10C, A14E, B1(N^{α}-carbamoyl), B3C, desB27, B29K(N^{ε}eicosanedioyl-gGlu-2×OEG), desB30 human insulin

A1(N^{α}-Glutaryl), A10C, A14E, B1(N^{α}-glutaryl), B3C, desB27, B29K(N^{ε}octadecanedioyl-gGlu-2×OEG), desB30 human insulin

A1(N^{α}-Glutaryl), A10C, A14E, B1(N^{α}-glutaryl), B3C, desB27, B29K(N^{ε}octadecanedioyl-gGlu), desB30 human insulin

A1(N^{α}-Glutaryl), A10C, A14E, B1(N^{α}-glutaryl), B3C, desB27, B29K(N^{ε}eicosanedioyl-gGlu), desB30 human insulin

A1(N^{α}-Glutaryl), A10C, A14E, B1(N^{α}-glutaryl), B3C, desB27, B29K(N^{ε}eicosanedioyl-gGlu-2×OEG), desB30 human insulin

A1(N^{α}-Diglycolyl), A10C, A14E, B1(N^{α}-diglycolyl), B3C, desB27, B29K(N^{ε}octadecanedioyl-gGlu-2×OEG), desB30 human insulin

A1(N^{α}-Diglycolyl), A10C, A14E, B1(N^{α}-diglycolyl), B3C, desB27, B29K(N^{ε}octadecanedioyl-gGlu), desB30 human insulin

A1(N^{α}-Diglycolyl), A10C, A14E, B1(N^{α}-diglycolyl), B3C, desB27, B29K(N^{ε}eicosanedioyl-gGlu), desB30 human insulin

A1(N^{α}-Diglycolyl), A10C, A14E, B1(N^{α}-diglycolyl), B3C, desB27, B29K(N^{ε}eicosanedioyl-gGlu-2×OEG), desB30 human insulin

52. An N-terminally modified insulin according to any one of the preceding aspects to the extent possible, which is selected from the group consisting of:

A1(N^{α},N^{α}-Dimethyl), A10C, A14E, B1(N^{α},N^{α}-dimethyl), B3C, B16H, B25H, B29K(N^{ε}eicosanedioyl-gGlu), desB30 human insulin

A1(N^{α},N^{α}-Dimethyl), A10C, A14E, B1G(N^{α},N^{α}-dimethyl), B3C, B16H, B25H, B29K(N^{ε}eicosanedioyl-gGlu), desB30 human insulin

A1(N^{α},N^{α}-Dimethyl), A10C, A14E, B1(N^{α},N^{α}-dimethyl), B3C, B16H, B25H, B29K(N^{ε}eicosanedioyl-gGlu-2×OEG), desB30 human insulin

A1(N^{α},N^{α}-Dimethyl), A10C, A14E, B1G(N^{α},N^{α}-dimethyl), B3C, B16H, B25H, B29K(N^{ε}eicosanedioyl-gGlu-2×OEG), desB30 human insulin

A1(N^{α}-Carbamoyl), A10C, A14E, B1(N^{α}-carbamoyl), B3C, B16H, B25H, B29K(N^{ε}eicosanedioyl-gGlu), desB30 human insulin

A1(N^{α}-Carbamoyl), A10C, A14E, B1(N^{α}-carbamoyl), B3C, B16H, B25H, B29K(N^{ε}eicosanedioyl-gGlu-2×OEG), desB30 human insulin

A1(N^{α}-Acetyl), A10C, A14E, B1(N^{α}-acetyl), B3C, B16H, B25H, B29K(N^{ε}eicosanedioyl-gGlu), desB30 human insulin

A1(N^{α}-Acetyl), A10C, A14E, B1(N^{α}-acetyl), B3C, B16H, B25H, B29K(N^{ε}eicosanedioyl-gGlu-2×OEG), desB30 human insulin

A1(N^{α}-Glutaryl), A10C, A14E, B1(N^{α}-glutaryl), B3C, B16H, B25H, B29K(N^{ε}eicosanedioyl-gGlu), desB30 human insulin

A1(N^{α}-Glutaryl), A10C, A14E, B1(N^{α}-glutaryl), B3C, B16H, B25H, B29K(N^{ε}eicosanedioyl-gGlu-2×OEG), desB30 human insulin

A1(N^{α}-Diglycolyl), A10C, A14E, B1(N^{α}-diglycolyl), B3C, B16H, B25H, B29K(N^{ε}eicosanedioyl-gGlu), desB30 human insulin

A1(N^{α}-Diglycolyl), A10C, A14E, B1(N^{α}-diglycolyl), B3C, B16H, B25H, B29K(N^{ε}eicosanedioyl-gGlu-2×OEG), desB30 human insulin

53. An N-terminally modified insulin according to any one of the preceding aspects to the extent possible, which is selected from the group consisting of:

A1(N^{α},N^{α}-Dimethyl), A10C, A14E, B1(N^{α},N^{α}-dimethyl), B3C, B16H, B25H, B29K(N^{ε}eicosanedioyl-gGlu), desB30 human insulin

A1(N^{α},N^{α}-Dimethyl), A10C, A14E, B1G(N^{α},N^{α}-dimethyl), B3C, B16H, B25H, B29K(N^{ε}eicosanedioyl-gGlu), desB30 human insulin

A1(N^{α},N^{α}-Dimethyl), A10C, A14E, B1(N^{α},N^{α}-dimethyl), B3C, B16H, B25H, B29K(N^{ε}eicosanedioyl-gGlu-2×OEG), desB30 human insulin

A1(N^{α},N^{α}-Dimethyl), A10C, A14E, B1G(N^{α},N^{α}-dimethyl), B3C, B16H, B25H, B29K(N^{ε}eicosanedioyl-gGlu-2×OEG), desB30 human insulin

54. An N-terminally modified insulin according to any one of the preceding aspects to the extent possible, which is selected from the group consisting of:

A1(N^{α},N^{α}-Dimethyl), A10C, A14E, B1(N^{α},N^{α}-dimethyl), B3C, B16H, B25H, B29K(N^{ε}eicosanedioyl-gGlu-2×OEG), desB30 human insulin

A1(N^{α},N^{α}-Dimethyl), A10C, A14E, B1G(N^{α},N^{α}-dimethyl), B3C, B16H, B25H, B29K(N^{ε}eicosanedioyl-gGlu-2×OEG), desB30 human insulin

55. An N-terminally modified insulin according to any one of the preceding aspects to the extent possible, which is selected from the group consisting of:

A1(N^{α},N^{α}-Dimethyl), A10C, A14E, B1(N^{α},N^{α}-dimethyl), B3C, B25H, desB27, B29K(N^{ε}octadecanedioyl-gGlu), desB30 human insulin

A1(N^{α},N^{α}-Dimethyl), A10C, A14E, B1G(N^{α},N^{α}-dimethyl), B3C, B25H, desB27, B29K(N^{ε}octadecanedioyl-gGlu), desB30 human insulin

A1(N^{α},N^{α}-Dimethyl), A10C, A14E, B1(N^{α},N^{α}-dimethyl), B3C, B25H, desB27, B29K(N^{ε}octadecanedioyl-gGlu-2×OEG), desB30 human insulin

A1(N^{α},N^{α}-Dimethyl), A10C, A14E, B1G(N^{α},N^{α}-dimethyl), B3C, B25H, desB27, B29K(N^{ε}octadecanedioyl-gGlu-2×OEG), desB30 human insulin

A1(N^{α},N^{α}-Dimethyl), A10C, A14E, B1(N^{α},N^{α}-dimethyl), B3C, B25H, desB27, B29K(N^{ε}eicosanedioyl-gGlu), desB30 human insulin

A1(N^{α},N^{α}-Dimethyl), A10C, A14E, B1G(N^{α},N^{α}-dimethyl), B3C, B25H, desB27, B29K(N^{ε}eicosanedioyl-gGlu), desB30 human insulin

A1(N^{α},N^{α}-Dimethyl), A10C, A14E, B1(N^{α},N^{α}-dimethyl), B3C, B25H, desB27, B29K(N^{ε}eicosanedioyl-gGlu-2×OEG), desB30 human insulin

A1(N^{α},N^{α}-Dimethyl), A10C, A14E, B1G(N^{α},N^{α}-dimethyl), B3C, B25H, desB27, B29K(N^{ε}eicosanedioyl-gGlu-2×OEG), desB30 human insulin

56. An N-terminally modified insulin according to any one of the preceding aspects to the extent possible which is any one of the compounds mentioned specifically in the above specification.
57. A pharmaceutical composition comprising an N-terminally modified insulin according to any one of the preceding aspects to the extent possible.
58. A pharmaceutical composition according to aspect 57, which is an oral pharmaceutical composition.
59. An oral pharmaceutical composition comprising one or more lipids and an N-terminally modified insulin.
60. An oral pharmaceutical composition according to aspect 59, wherein the N-terminally modified insulin consists of a peptide part, an N-terminal modification group and albumin binding moiety.
61. An oral pharmaceutical composition according to any one of aspects 59-60, which is anhydrous.
62. An oral pharmaceutical composition according to any one of aspects 59-61, wherein the lipids are selected from the group consisting of: Glycerol mono-caprylate (such as e.g. Rylo MG08 Pharma) and Glycerol mono-caprate (such as e.g. Rylo MG10 Pharma from Danisco). In another aspect the lipid is selected from the group consisting of: propyleneglycol caprylate (such as e.g. Capmul PG8 from Abitec or Capryol PGMC, or Capryol 90 from Gattefosse).
63. An oral pharmaceutical composition according to any one of aspects 59-62, which is a solid or semi-solid pharmaceutical composition comprising an N-terminally modified insulin (a), at least one polar organic solvent (b) for the N-terminally modified insulin, at least one surfactant (c), at least one lipophilic component (d), and optionally at least one solid hydrophilic component (e), wherein said pharmaceutical composition is spontaneously dispersible.
64. An oral pharmaceutical composition according to any one of aspects 59-63, which is a water-free liquid pharmaceutical composition comprising an N-terminally modified insulin (a), at least one polar organic solvent (b) for the N-terminally modified insulin, at least one lipophilic component (c), and optionally at least one surfactant (d), wherein the pharmaceutical composition is in the form of a clear solution.
65. An oral pharmaceutical composition according to any one of aspects 59-64, wherein the surfactant is a non-ionic surfactant.
66. An oral pharmaceutical composition according to any one of aspects 59-65, wherein the surfactant is a solid surfactant selected from the group consisting of a poloxamer and a mixture of poloxamers such as Pluronic F-127 or Pluronic F-68.
67. An oral pharmaceutical composition according to any one of aspects 59-66, wherein the lipophilic component is a mono-di-glyceride.
68. An oral pharmaceutical composition according to any one of aspects 59-67, wherein the lipophilic component is chosen such that a solution is obtained when the lipophilic component is mixed with propylene glycol.
69. An oral pharmaceutical composition according to any one of aspects 59-68, wherein the lipophilic component is a mono- and/or di-glyceride or propylene glycol caprylate.
70. An oral pharmaceutical composition according to any one of aspects 59-69, which is a liquid pharmaceutical composition comprising at least one N-terminally modified insulin, at least one polar organic solvent and at least two non-ionic surfactants with HLB above 10, wherein the composition does not contain oil or any other lipid component or surfactant with an HLB below 7.
71. An oral pharmaceutical composition according to any one of aspects 59-70, wherein the composition forms a micro- or nanoemulsion after dilution in an aqueous medium.
72. An oral pharmaceutical composition according to any one of aspects 59-71, wherein the organic solvent is selected from the group consisting of polyols.
73. An oral pharmaceutical composition according to any one of aspects 59-72, wherein the organic solvent is selected from the group consisting of propylene glycol, glycerol and mixtures thereof.
74. An oral pharmaceutical composition according to any one of aspects 59-73, wherein the organic solvent is propylene glycol.
75. An oral pharmaceutical composition according to any one of aspects 59-74, wherein one or more of said non-ionic surfactants comprise a medium chain fatty acid group such as C8 fatty acids (caprylates), C10 fatty acids (caprates) or C12 fatty acids (laurates)
76. An oral pharmaceutical composition according to any one of aspects 59-75, wherein one or more of said non-ionic surfactants are selected from the group consisting of Labrasol (also named Caprylocaproyl Macrogolglycerides), Tween 20 (also named Polysorbate 20 or Polyethylene glycol sorbitan monolaurate), Tween 80 (also named polysorbate 80), Diglycerol monocaprylate, Polyglycerol caprylate and Cremophor RH 40.
77. An oral pharmaceutical composition according to any one of aspects 59-76, wherein the organic solvent is present in the amount from about 1% to about 15%.
78. An oral pharmaceutical composition according to any one of aspects 59-77, wherein the modification groups are one or two organic substituents which are each having a molecular weight (MW) below 200 g per mol conjugated to the N-terminal of the parent insulin.
79. An oral pharmaceutical composition according to any one of aspects 59-78, wherein modification groups are designated Y′ and Z in Chem. V:

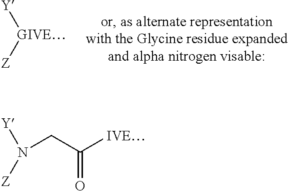

and wherein Y′ and Z are attached to the N-terminal amino acids of the insulin peptide.
80. An oral pharmaceutical composition according to any one of aspects 59-79, wherein Y′ and Z are different and
Y′ is R—C(═X)—,
Z is H,
R is H, NH₂, straight chain or branched C1-C4 alkyl, straight chain or branched C2-C4 alkylsubstituted with dimethylamino, diethylamino, dipropylamino, dimethylammonium, diethylammonium, dipropylammonium, CO₂H, or —OCH₂CO₂H, C5-C6 cycloalkyl, substituted C5-C6 cycloalkyl, 5- or 6-membered saturated heterocyclyl, substituted 5- or 6-membered saturated heterocyclyl, and
X is O or S.
   81. An oral pharmaceutical composition according to any one of aspects 59-79, wherein Y′ and Z are different and
Y′ is R—C(═X)—,
Z is H,
R is H, NH₂, straight chain or branched C1-C4 alkyl, C1-C4 alkyl substituted with CO₂H, or —OCH₂CO₂H, C5-C6 cycloalkyl, 5- or 6-membered saturated heterocyclyl, and
X is O or S.
   82. An oral pharmaceutical composition according to any one of aspects 59-79, wherein Y′ and Z are different and
Y′ is R—C(═X)—,
Z is H,
R is H, NH₂, straight chain or branched C1-C4 alkyl, C1-C4 alkyl substituted with CO₂H, or —OCH₂CO₂H, and
X is O.
   83. An oral pharmaceutical composition according to any one of aspects 59-79, wherein Y′ and Z are different and
Y′ is straight chain or branched C1-C4 alkyl, straight chain or branched C2-C4 acyl substituted with dimethylamino, diethylamino, dipropylamino, trimethylammonium, triethylammonium or dipropylammonium, 5- or 6-membered saturated heterocyclyl, substituted 5- or 6-membered saturated heterocyclyl, amidinyl, and
Z is H.
   84. An oral pharmaceutical composition according to any one of aspects 59-79, wherein Y′ and Z are different and
Y′ is straight chain C1-C4 alkyl, 5- or 6-membered saturated heterocyclyl, and
Z is H.
   85. An oral pharmaceutical composition according to any one of aspects 59-79, wherein Y′=Z=C1-C4 alkyl.
   86. An oral pharmaceutical composition according to any one of aspects 59-79, wherein Y′ and Z are the same and selected from the group consisting of: methyl, ethyl, n-propyl, sec-propyl, n-butyl and i-butyl.
   87. An oral pharmaceutical composition according to any one of aspects 59-79, wherein Y′ and Z are the same and selected from dimethyl and diethyl.
   88. An oral pharmaceutical composition according to any one of aspects 59-79, wherein Y′ and Z are the same and methyl.
   89. An oral pharmaceutical composition according to any one of aspects 59-79, wherein the N-terminal modification is positively charged at physiological pH.
   90. An oral pharmaceutical composition according to any one of aspects 59-79, wherein the N-terminal modification is selected from the group consisting of: N,N-di-C1-4alkyl, N-amidinyl, 4-(N,N-dimethylamino)butanoyl, 3-(1-piperidinyl)propionyl, 3-(N,N-dimethylamino)propionyl, and N,N-dimethyl-glycyl.
   91. An oral pharmaceutical composition according to any one of aspects 59-79, wherein the N-terminal modification is N,N-di-C1-4alkyl.
   92. An oral pharmaceutical composition according to any one of aspects 59-79, wherein the N-terminal modification is N,N-dimethyl or N,N-diethyl.
   93. An oral pharmaceutical composition according to any one of aspects 59-92, wherein the N-terminal modification group is not malonyl or succinyl.
   94. An oral pharmaceutical composition according to any one of aspects 59-92, wherein the N-terminal modification group is not malonyl.
   95. An oral pharmaceutical composition according to any one of aspects 59-92, wherein the N-terminal modification group is not succinyl.
   96. An oral pharmaceutical composition according to any one of aspects 59-79, wherein the N-terminal modification group is selected from the group consisting of: N,N-dimethyl, N,N-diethyl, carbamoyl, formyl, acetyl, propionyl, butyryl, glutaryl, and diglycolyl.
   97. An oral pharmaceutical composition according to any one of aspects 59-79, wherein the N-terminal modification is selected from the group consisting of: Carbamoyl, thiocarbamoyl, short chain acyl groups, oxalyl, glutaryl and diglycolyl.
   98. An oral pharmaceutical composition according to any one of aspects 59-79, wherein the N-terminal modification is selected from the group consisting of: Carbamoyl, thiocarbamoyl, formyl, acetyl, propionyl, butyryl, pyroglutamyl, oxalyl, glutaryl and diglycolyl.
   99. An oral pharmaceutical composition according to any one of aspects 59-79, wherein the N-terminal modification is neutral at physiological pH.
   100. An oral pharmaceutical composition according to any one of aspects 59-79, wherein the N-terminal modification is selected from the group consisting of: Carbamoyl, thiocarbamoyl, formyl, acetyl, propionyl, butyryl, and pyroglutamyl.
   101. An oral pharmaceutical composition according to any one of aspects 59-79, wherein the N-terminal modification is selected from the group consisting of: Carbamoyl, acetyl, propionyl, and butyryl.
   102. An oral pharmaceutical composition according to any one of aspects 59-79, wherein the N-terminal modification is carbamoyl.
   103. An oral pharmaceutical composition according to any one of aspects 59-79, wherein the N-terminal modification is acetyl.
   104. An oral pharmaceutical composition according to any one of aspects 59-79, wherein the N-terminal modification is negatively charged at physiological pH.
   105. An oral pharmaceutical composition according to any one of aspects 59-79, wherein the N-terminal modification is selected from the group consisting of: oxalyl, glutaryl and diglycolyl.
   106. An oral pharmaceutical composition according to any one of aspects 59-79, wherein the N-terminal modification is glutaryl.
   107. An oral pharmaceutical compositions according to any one of aspects 59-79, wherein the N-terminal modification is diglycolyl.
   108. An oral pharmaceutical composition according to any one of aspects 59-107, wherein the peptide part is human insulin substituted with at least two cysteines in positions which are selected from the group consisting of:

A10C, B1C;

A10C, B2C;

A10C, B3C;

A10C, B4C;

A10C, B5C; and

B1C, B4C.

109. An oral pharmaceutical composition according to any one of aspects 59-108, wherein the positions for cysteine substitution are selected from the group consisting of:

A10C, B1C;

A10C, B2C;

A10C, B3C;

A10C, B4C; and

B1C, B4C.

110. An oral pharmaceutical composition according to any one of aspects 59-109, wherein the positions for cysteine substitution are selected from the group consisting of:

A10C, B1C;

A10C, B2C;

A10C, B3C; and

A10C, B4C.

111. An oral pharmaceutical composition according to any one of aspects 59-110, wherein the positions for cysteine substitution are selected from the group consisting of:

A10C, B3C; and

A10C, B4C.

112. An oral pharmaceutical composition according to any one of aspects 59-111, wherein the positions for cysteine substitution are A10C and B3C.
113. An oral pharmaceutical composition according to any one of aspects 59-112, which further comprises mutations such that at least one hydrophobic amino acid has been substituted with hydrophilic amino acids, and wherein said substitution is within or in close proximity to one or more protease cleavage sites of the insulin.
114. An oral pharmaceutical composition according to any one of aspects 59-113, wherein the peptide part in addition to two or more cysteine substitutions comprises one or more substituted amino acids selected from the group consisting of: A8H, A14E, A14H, A21G, B1G, B3Q, B3E, B3T, B3V, B3L, B16H, B16E, B25A, B25H, B25N, B27E, B27P, B28E, desB1, desB27 and desB30.
115. An oral pharmaceutical composition according to any one of aspects 59-113, wherein the peptide part in addition to two or more cysteine substitutions comprises one or more substituted amino acids selected from the group consisting of: A14E, A14H, A21G, desB1, B1G, B3Q, B3E, B16H, B16E, B25H, desB27, and desB30.
116. An oral pharmaceutical composition according to any one of aspects 59-113, wherein the peptide part in addition to two or more cysteine substitutions comprises one or more substituted amino acids selected from the group consisting of: A14E, desB1, B1G, B16H, B16E, B25H, desB27 and desB30.
117. An oral pharmaceutical composition according to any one of aspects 59-113, wherein the peptide part in addition to two or more cysteine substitutions comprises one or more substituted amino acids selected from the group consisting of: A14E, B16H, B25H, desB27, and desB30.
118. An oral pharmaceutical composition according to any one of aspects 59-117, wherein the peptide part is selected from the group consisting of: A10C, A14E, B1C, B16H, B25H, desB30 human insulin; A10C, A14E, B1C, B25H, desB30 human insulin; A10C, A14E, B2C, B16H, B25H, desB30 human insulin; A10C, A14E, B2C, B25H, desB30 human insulin; A10C, A14E, B3C, B16H, B25H, desB30 human insulin; A10C, A14E, B3C, B25H, desB27, desB30 human insulin; A10C, A14E, B3C, B25H, desB30 human insulin; A10C, A14E, B3C, desB27, desB30 human insulin; A10C, A14E, B3C, B16H, B25H, desB27, desB30 human insulin; A10C, A14E, B16H, desB27, desB30 human insulin; A10C, A14E, B4C, B16H, B25H, desB30 human insulin; A10C, A14E, B4C, B25A, desB30 human insulin; A10C, A14E, B4C, B25H, B28E, desB30 human insulin; A10C, A14E, B4C, B25H, desB27, desB30 human insulin; A10C, A14E, B4C, B25H, desB30 human insulin; A10C, A14E, B4C, B25N, B27E, desB30 human insulin; A10C, A14E, B4C, B25N, desB27, desB30 human insulin; A10C, A14E, desB1, B4C, B25H, desB30 human insulin; A10C, A14H, B4C, B25H, desB30 human insulin; A10C, A14E, B1C, B16H, B25H, desB30 human insulin; A10C, A14E, B2C, B16H, B25H, desB30 human insulin; and A10C, A14E, B4C, B16H, B25H, desB30 human insulin.
119. An oral pharmaceutical composition according to any one of aspects 59-117, wherein the peptide part is selected from the group consisting of: A10C, A14E, B3C, B16H, B25H, desB30 human insulin; A10C, A14E, B3C, B25H, desB27, desB30 human insulin; A10C, A14E, B3C, B25H, desB30 human insulin; A10C, A14E, B3C, desB27, desB30 human insulin; A10C, A14E, B3C, B16H, B25H, desB27, desB30 human insulin; A10C, A14E, B16H, desB27, desB30 human insulin.
120. An oral pharmaceutical composition according to any one of aspects 59-117, wherein the peptide part is selected from the group consisting of: A10C, A14E, B1C, B16H, B25H, desB30 human insulin; A10C, A14E, B1C, B25H, desB30 human insulin; A10C, A14E, B2C, B16H, B25H, desB30 human insulin; A10C, A14E, B2C, B25H, desB30 human insulin; A10C, A14E, B3C, B16H, B25H, desB30 human insulin; A10C, A14E, B3C, B25H, desB27, desB30 human insulin; A10C, A14E, B3C, B25H, desB30 human insulin; A10C, A14E, B3C, desB27, desB30 human insulin; A10C, A14E, B3C, B16H, B25H, desB27, desB30 human insulin; A10C, A14E, B16H, desB27, desB30 human insulin; A10C, A14E, B4C, B16H, B25H, desB30 human insulin; A10C, A14E, B4C, B25A, desB30 human insulin; A10C, A14E, B4C, B25H, B28E, desB30 human insulin; A10C, A14E, B4C, B25H, desB27, desB30 human insulin; A10C, A14E, B4C, B25H, desB30 human insulin; A10C, A14E, B4C, B25N, B27E, desB30 human insulin; A10C, A14E, B4C, B25N, desB27, desB30 human insulin; A10C, A14E, desB1, B4C, B25H, desB30 human insulin; A10C, A14H, B4C, B25H, desB30 human insulin; A10C, A14E, B1C, B16H, B25H, desB30 human insulin; A10C, A14E, B2C, B16H, B25H, desB30 human insulin; and A10C, A14E, B4C, B16H, B25H, desB30 human insulin.
121. An oral pharmaceutical composition according to any one of aspects 59-117, wherein the peptide part is selected from the group consisting of: A10C, A14E, B3C, B16H, B25H, desB30 human insulin; A10C, A14E, B3C, B25H, desB27, desB30 human insulin; A10C, A14E, B3C, B25H, desB30 human insulin; A10C, A14E, B3C, desB27, desB30 human insulin; A10C, A14E, B3C, B16H, B25H, desB27, desB30 human insulin; A10C, A14E, B16H, desB27, desB30 human insulin.
122. An oral pharmaceutical composition according to any one of aspects 59-121, wherein the albumin binding moiety is a side chain consisting of a fatty acid or a fatty diacid attached to the insulin, optionally via a linker, in an amino acid position of the peptide part.
123. An oral pharmaceutical composition according to any one of aspects 59-122, wherein the peptide part comprises only one lysine residue and the albumin binding moiety is attached, optionally via a linker, to said lysine residue.
124. An oral pharmaceutical composition according to any one of aspects 59-123, wherein the albumin binding moiety has the general formula Acy-AA1ₙ-AA2ₘ-AA3ₚ- (Chem. IV), wherein

n is 0 or an integer in the range from 1 to 3;

m is 0 or an integer in the range from 1 to 10;

p is 0 or an integer in the range from 1 to 10;

Acy is a fatty acid or a fatty diacid comprising from about 14 to about 20 carbon atoms;

AA1 is a neutral linear or cyclic amino acid residue;

AA2 is an acidic amino acid residue;

AA3 is a neutral, alkyleneglycol-containing amino acid residue;

the order by which AA1, AA2 and AA3 appears in the formula can be interchanged independently; AA2 can occur several times along the formula (e.g., Acy-AA2-AA3₂-AA2-); AA2 can occur independently (=being different) several times along the formula (e.g., Acy-AA2-AA3₂-AA2-); the connections between Acy, AA1, AA2 and/or AA3 are amide (peptide) bonds which, formally, can be obtained by removal of a hydrogen atom or a hydroxyl group (water) from each of Acy, AA1, AA2 and AA3; and attachment to the peptide part can be from the C-terminal end of a AA1, AA2, or AA3 residue in the acyl moiety of the Chem. IV or from one of the side chain(s) of an AA2 residue present in the moiety of Chem. IV.
125. A method of producing an N-terminally modified insulin derivative according to any one of aspects 1-56.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### EXAMPLES

The following examples are offered by way of illustration, not by limitation.

The abbreviations used herein are the following: βAla is beta-alanyl, Aoc is 8-aminooctanoic acid, tBu is tert-butyl, CV is column volumes, DCM is dichloromethane, DIC is diisopropylcarbodiimide, DIPEA=DIEA is N,N-disopropylethylamine, DMF is N,N-dimethylformamide, DMSO is dimethyl sulphoxide, EtOAc is ethyl acetate, Fmoc is 9-fluorenyl-methyloxycarbonyl, γGlu is gamma L-glutamyl, HCl is hydrochloric acid, HOBt is 1-hydroxybenzotriazole, NMP is N-methylpyrrolidone, MeCN is acetonitrile, OEG is [2-(2-aminoethoxy)ethoxy]ethylcarbonyl, Su is succinimidyl-1-yl=2,5-dioxo-pyrrolidin-1-yl, OSu is succinimidyl-1-yloxy=2,5-dioxo-pyrrolidin-1-yloxy, RPC is reverse phase chromatography, RT is room temperature, TFA is trifluoroacetic acid, THF is tetrahydrofuran, TNBS is 2,4,6-trinitro-benzenesulfonic acid, TRIS is tris(hydroxymethyl)aminomethane and TSTU is O—(N-succinimidyl)-1,1,3,3-tetramethyluronium tetrafluoroborate.

The following examples and general procedures refer to intermediate compounds and final products identified in the specification and in the synthesis schemes. The preparation of the compounds of the present invention is described in detail using the following examples, but the chemical reactions described are disclosed in terms of their general applicability to the preparation of compounds of the invention. Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, that is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents.

The compounds of the invention can be purified by employing one or more of the following procedures which are typical within the art. These procedures can—if needed—be modified with regard to gradients, pH, salts, concentrations, flow, columns and so forth. Depending on factors such as impurity profile, solubility of the insulins in question etcetera, these modifications can readily be recognised and made by a person skilled in the art.

After acidic HPLC or desalting, the compounds are isolated by lyophilisation of the pure fractions.

After neutral HPLC or anion exchange chromatography, the compounds are desalted, precipitated at isoelectrical pH, or purified by acidic HPLC.

Typical Purification Procedures:

The HPLC system is a Gilson system consisting of the following: Model 215 Liquid handler, Model 322-H2 Pump and a Model 155 UV Dector. Detection is typically at 210 nm and 280 nm.

The Äkta Purifier FPLC system (GE Health Care) consists of the following: Model P-900 Pump, Model UV-900 UV detector, Model pH/C-900 pH and conductivity detector, Model Frac-950 Fraction collector. UV detection is typically at 214 nm, 254 nm and 276 nm. The Äkta Explorer Air FPLC system (Amersham BioGE Health Caresciences) consists of the following: Model P-900 Pump, Model UV-900 UV detector, Model pH/C-900 pH and conductivity detector, Model Frac-950 Fraction collector. UV detection is typically at 214 nm, 254 nm and 276 nm

Acidic HPLC:
Column: Phenomenex, Gemini, 5μ, C18, 110 Å, 250×30 cm
Flow: 20 ml/min′
Eluent: A: 0.1% TFA in water B: 0.1% TFA in CH₃CN
Gradient:

| | | |
|---|---|---|
| 0-7.5 | min: | 10% B |
| 7.5-87.5 | min: | 10% B to 60% B |
| 87.5-92.5 | min: | 60% B |
| 92.5-97.5 | min: | 60% B to 100% B |

Neutral HPLC:
Column: Phenomenex, Gemini, C18, 5 μm 250×30.00 mm, 110 Å
Flow: 20 ml/min
Eluent: A: 20% CH₃CN in aqueous 10 mM TRIS+15 mM (NH₄)SO₄ pH=7.3 B: 80% CH₃CN, 20% water

Gradient:

| | | |
|---|---|---|
| 0-7.5 | min: | 0% B |
| 7.5-52.5 | min: | 0% B to 60% B |
| 52.5-57.5 | min: | 60% B |
| 57.5-58 | min: | 60% B to 100% B |
| 58-60 | min: | 100% B |
| 60-63 | min: | 10% B |

Anion exchange chromatography:
Column: RessourceQ, 6 ml,
Flow: 6 ml/min
Buffer A: 0.09% NH₄HCO₃, 0.25% NH₄OAc, 42.5% ethanol pH 8.4
Buffer B: 0.09% NH₄HCO₃, 2.5% NH₄OAc, 42.5% ethanol pH 8.4
Gradient: 100% A to 100% B during 30 CV
Column: Source 30Q, 30×250 mm
Flow: 80 ml/min
Buffer A: 15 mM TRIS, 30 mM Ammoniumacetat i 50% Ethanol, pH 7.5 (1.25 mS/cm)
Buffer B: 15 mM TRIS, 300 mM Ammoniumacetat i 50% Ethanol pH 7.5 (7.7 mS/cm)
Gradient: 15% B to 70% B over 40 CV

Desalting:
Column: Daiso 200 Å 15 um FeFgel 304, 30×250 mm
Buffer A: 20 v/v % Ethanol, 0.2% acetic acid
Buffer B: 80% v/v % Ethanol, 0.2% acetic acid
Gradient: 0-80% B over 1.5 CV
Flow: 80 ml/min
Column: HiPrep 2610
Flow: 10 ml/min,
Gradient: 6 CV
Buffer: 10 mM NH₄HCO₃
   General procedure for the solid phase synthesis of acylation reagents of the general formula (II):

Acy-AA1ₙ-AA2ₘ-AA3ₚ-Act, (II):

wherein Acy, AA1, AA2, AA3, n, m, and p are as defined above and Act is the leaving group of an active ester, such as N-hydroxysuccinimide (OSu), or 1-hydroxybenzotriazole, and

wherein carboxylic acids within the Acy and AA2 moieties of the acyl moiety are protected as tert-butyl esters.

Compounds of the general formula (II) according to the invention can be synthesised on solid support using procedures well known to skilled persons in the art of solid phase peptide synthesis. This procedure comprises attachment of a Fmoc protected amino acid to a polystyrene 2-chlorotritylchloride resin. The attachment can, e.g., be accomplished using the free N-protected amino acid in the presence of a tertiary amine, like triethyl amine or N,N-diisopropylethylamine (see references below). The C-terminal end (which is attached to the resin) of this amino acid is at the end of the synthetic sequence being coupled to the parent insulins of the invention. After attachment of the Fmoc amino acid to the resin, the Fmoc group is deprotected using, e.g., secondary amines, like piperidine or diethyl amine, followed by coupling of another (or the same) Fmoc protected amino acid and deprotection. The synthetic sequence is terminated by coupling of mono-tert-butyl protected fatty (α, ω) diacids, like hexadecanedioic, heptadecanedioic, octadecanedioic or eicosanedioic acid mono-tert-butyl esters. Cleavage of the compounds from the resin is accomplished using diluted acid like 0.5-5% TFA/DCM (trifluoroacetic acid in dichloromethane), acetic acid (e.g., 10% in DCM, or HOAc/triflouroethanol/DCM 1:1:8), or hecafluoroisopropanol in DCM (See, e.g., “Organic Synthesis on Solid Phase”, F. Z. Dörwald, Wiley-VCH, 2000. ISBN 3-527-29950-5, “Peptides: Chemistry and Biology”, N. Sewald & H.-D. Jakubke, Wiley-VCH, 2002, ISBN 3-527-30405-3 or “The Combinatorial Chemistry Catalog” 1999, Novabiochem AG, and references cited therein). This ensures that tert-butyl esters present in the compounds as carboxylic acid protecting groups are not deprotected. Finally, the C-terminal carboxy group (liberated from the resin) is activated, e.g., as the N-hydroxysuccinimide ester (OSu) and used either directly or after purification as coupling reagent in attachment to parent insulins of the invention. This procedure is described in example 9 in WO09115469.

Alternatively, the acylation reagents of the general formula (II) above can be prepared by solution phase synthesis as described below.

Mono-tert-butyl protected fatty diacids, such as hexadecanedioic, heptadecanedioic, octadecanedioic or eicosanedioic acid mono-tert-butyl esters are activated, e.g., as OSu-esters as described below or as any other activated ester known to those skilled in the art, such as HOBt- or HOAt-esters. This active ester is coupled with one of the amino acids AA1, mono-tert-butyl protected AA2, or AA3 in a suitable solvent such as THF, DMF, NMP (or a solvent mixture) in the presence of a suitable base, such as DIPEA or triethylamine. The intermediate is isolated, e.g., by extractive procedures or by chromatographic procedures. The resulting intermediate is again subjected to activation (as described above) and to coupling with one of the amino acids AA1, mono-tert-butyl protected AA2, or AA3 as described above. This procedure is repeated until the desired protected intermediate Acy-AA1ₙ-AA2ₘ-AA3ₚ-OH is obtained. This is in turn activated to afford the acylation reagents of the general formula (II) Acy-AA1ₙ-AA2ₘ-AA3ₚ-Act. This procedure is described in example 11 in WO09115469.

The acylation reagents prepared by any of the above methods can be (tert-butyl) deprotected after activation as OSu esters. This can be done by TFA treatment of the OSu-activated tert-butyl protected acylation reagent. After acylation of any insulin, the resulting unprotected acylated protease stabilized insulin of the invention is obtained. This procedure is described in example 16 in WO09115469.

If the reagents prepared by any of the above methods are not (tert-butyl) deprotected after activation as OSu esters, acylation of any insulin affords the corresponding tert-butyl protected acylated insulin of the invention. In order to obtain the unprotected acylated insulin of the invention, the protected insulin is to be de-protected. This can be done by TFA treatment to afford the unprotected acylated insulin of the invention. This procedure is described in example 1 in WO05012347.

Methods for preparation of acylated insulins without N-terminal protection (i.e. starting materials for preparation of N-terminally protected derivatives of the invention) can be found in WO09115469.

General Procedure (A) for Preparation for Reductive N-Methylation of Acylated Insulins of this Invention

The acylated insulin is dissolved in a mixture of a polar aprotic or protic solvent, such as N-methylformamide, DMF, NMP, THF or DMSO (3.8 ml) and water optionally containing a buffer such as 0.2 M citrate buffer, sodium acetate buffer or diluted acetic acid at acidic pH (around 4, wide tolerance), and the mixture is gently stirred. 37% Aqueous formaldehyde solution (5-10 or more equivalents)—or acetaldehyde, if N,N-diethyl derivatives are desired—is added, followed by addition of a freshly prepared solution of either sodium cyanoborohydride (5-10 equivalents) in methanol or water or 2-.picoline borane in THF or NMP or the like. The mixture is gently stirred. After completion of the reaction, the mixture is carefully acidified by dropwise addition of 1 N hydrochloric acid to pH 2-3. The product is isolated by preparative HPLC or anion exchange chromatography (AIEC).

The general procedure (A) is illustrated in example 1.

### Example 1

### General Procedure (A)

A1(N^{α},N^{α}-Dimethyl), A10C, A14E, B1(N^{α},N^{α}-dimethyl), B3C, B25H, desB27, B29K(N^{ε}octadecanedioyl-gGlu), desB30 human insulin
IUPAC (OpenEye, IUPAC style) name:

N{A1},N{A1}-dimethyl, N{B1},N{B1}-dimethyl, N{Epsilon-B29}-[(4S)-4-carboxy-4-(17-carboxyheptadecanoylamino)butanoyl]-[CysA10, GluA14, CysB3, HisB25], des-ThrB27, ThrB30-Insulin (human).

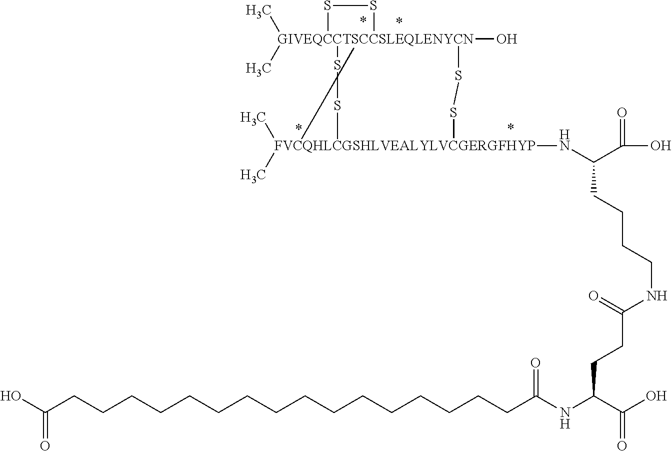

A10C, A14E, B3C, B25H, desB27, B29K(N^{ε}Octadecanedioyl-gGlu), desB30 human insulin (0.35 g) was dissolved in water (20 mL) and THF (10 mL). pH was adjusted to 3.9 with 1N NaOH. To this solution aqueous formaldehyde (37%, 49 μL) was added followed by slow dropwise addition of a solution of 2-picoline borane in THF (0.5 mL). The resulting mixture was left at room temperature for 5 hours, and overnight at 5° C. The mixture was added 30% acetonitrile in water to double volume and pH was adjusted to 2.5 with 1N hydrochloric acid.

The derivative was purified by preparative HPLC:
Column: Phenomenex, Gemini, 5μ, C18, 110 Å, Axia 250×30 cm
Flow: 25 ml/min′
Eluent:
   A: 0.1% TFA in water containing 10% acetonitrile
   B: 0.1% TFA in water containing 60% acetonitrile
Gradient: 20-100% B over 40 minutes

Pure fractions were pooled and lyophilized. The dry material was dissolved in water containing 40% acetonitrile (50 mL) and added 1 N NaOH to pH=8 and lyophilised to afford 0.28 g of the title insulin derivative.

LC-MS (electrospray): (m+4)/4: 1506.2 (6020.8)

Similarly, the following derivatives were prepared:

### Example 2

### General Procedure (A)

### A1(N^{α},N^{α}-Dimethyl), A10C, A14E, B1(N^{α},N^{α}-dimethyl), B3C, B25H, B29K(N^{ε}eicosanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[CysA10, GluA14, CysB3, HisB25], des-ThrB30-Insulin (human)

LC-MS (electrospray): m/z=1610.91 ((m+4)/4). Calcd.: 1610.89

### Example 3

### General Procedure (A)

### A1(N^{α},N^{α}-Dimethyl), A10C, A14E, B1(N^{α},N^{α}-dimethyl), B3C, B25H, B29K(N^{ε}octadecanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(17-carboxyheptadecanoylamino)butanoyl]-[CysA10,GluA14,CysB3,HisB25], des-ThrB30-Insulin (human).

LC-MS (electrospray): m/z=1531.35 ((m+4)/4). Calcd.: 1531.29

Similarly, the following insulins of the invention may be prepared:

### Example 4

### General Procedure (A)

### A1(N^{α},N^{α}-Dimethyl), A10C, A14E, B1(N^{α},N^{α}-dimethyl), B3C, B16H, B25H, B29K(N^{ε}octadecanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(17-carboxyheptadecanoylamino)butanoyl]-[CysA10,GluA14,CysB3,HisB16,HisB25], des-ThrB30-Insulin (human).

### Example 5

### General Procedure (A)

### A1(N^{α},N^{α}-Dimethyl), A10C, A14E, B1(N^{α},N^{α}-dimethyl), B3C, desB27, B29K(N^{ε}octadecanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(17-carboxyheptadecanoylamino)butanoyl]-[CysA10,GluA14,CysB3], des-ThrB27,ThrB30-Insulin (human).

### Example 6

### General Procedure (A)

### A1(N^{α},N^{α}-Dimethyl), A10C, A14E, B1G(N^{α},N^{α}-dimethyl), B3C, B25H, B29K(N^{ε}octadecanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(17-carboxyheptadecanoylamino)butanoyl]-[CysA10,GluA14,GlyB1,CysB3,HisB25], des-ThrB30-Insulin (human).

### Example 7

### General Procedure (A)

### A1(N^{α},N^{α}-Dimethyl), A10C, A14E, B1G(N^{α},N^{α}-dimethyl), B3C, B25H, desB27, B29K(N^{ε}octadecanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(17-carboxyheptadecanoylamino)butanoyl]-[CysA10,GluA14,GlyB1,CysB3,HisB25], des-ThrB27,ThrB30-Insulin (human).

### Example 8

### General Procedure (A)

### A1(N^{α},N^{α}-Dimethyl), A10C, A14E, B1G(N^{α},N^{α}-dimethyl), B3C, B16H, B25H, B29K(N^{ε}octadecanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(17-carboxyheptadecanoylamino)butanoyl]-[CysA10,GluA14,GlyB1,CysB3,HisB16,HisB25], des-ThrB30-Insulin (human).

### Example 9

### General Procedure (A)

### A1(N^{α},N^{α}-Dimethyl), A10C, A14E, B1G(N^{α},N^{α}-dimethyl), B3C, desB27, B29K(N^{ε}octadecanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(17-carboxyheptadecanoylamino)butanoyl]-[CysA10,GluA14,GlyB1,CysB3], des-ThrB27,ThrB30-Insulin (human).

### Example 10

### General Procedure (A)

### A1(N^{α},N^{α}-Dimethyl), A10C, A14E, B1(N^{α},N^{α}-dimethyl), B3C, desB27, B29K(N^{ε}octadecanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[CysA10,GluA14,CysB3], des-ThrB27,ThrB30-Insulin (human)

### Example 11

### General Procedure (A)

A1(N^{α},N^{α}-Dimethyl), A10C, A14E, B1(N^{α},N^{α}-dimethyl), B3C, B25H, desB27, B29K(N^{ε}octadecanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[CysA10,GluA14,CysB3,B25H], des-ThrB27,ThrB30-Insulin (human)

### Example 12

### General Procedure (A)

### A1(N^{α},N^{α}-Dimethyl), A10C, A14E, B1(N^{α},N^{α}-dimethyl), B3C, B25H, B29K(N^{ε}octadecanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[CysA10,GluA14,CysB3,B25H], des-ThrB30-Insulin (human)

### Example 13

### General Procedure (A)

A1(N^{α},N^{α}-Dimethyl), A10C, A14E, B1(N^{α},N^{α}-dimethyl), B3C, B16H, B25H, B29K(N^{ε}octadecanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[CysA10,GluA14,CysB3,B16H,B25H], des-ThrB30-Insulin (human)

### Example 14

### General Procedure (A)

### A1(N^{α},N^{α}-Dimethyl), A10C, A14E, B1G(N^{α},N^{α}-dimethyl), B3C, desB27, B29K(N^{ε}octadecanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[CysA10,GluA14,GlyB1,CysB3], des-ThrB27,ThrB30-Insulin (human)

### Example 15

### General Procedure (A)

### A1(N^{α},N^{α}-Dimethyl), A10C, A14E, B1G(N^{α},N^{α}-dimethyl), B3C, B25H, desB27, B29K(N^{ε}octadecanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[CysA10,GluA14,GlyB1,CysB3,B25H], des-ThrB27,ThrB30-Insulin (human)

### Example 16

### General Procedure (A)

### A1(N^{α},N^{α}-Dimethyl), A10C, A14E, B1G(N^{α},N^{α}-dimethyl), B3C, B25H, B29K(N^{ε}octadecanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(17-carboxyheptadecanoylam ino)butanoyl]amino]ethoxy]ethoxy]acetyl]amino]-ethoxy]ethoxy]acetyl]-[CysA10,GluA14,GlyB1,CysB3,B25H], des-ThrB30-Insulin (human)

### Example 17

### General Procedure (A)

### A1(N^{α},N^{α}-Dimethyl), A10C, A14E, B1G(N^{α},N^{α}-dimethyl), B3C, B16H, B25H, B29K(N^{ε}octadecanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[CysA10,GluA14,GlyB1,CysB3,B16H,B25H], des-ThrB30-Insulin (human)

The following derivative was prepared similarly as described above:

### Example 18

### General Procedure (A)

### A1(N^{α},N^{α}-Dimethyl), A10C, A14E, B1(N^{α},N^{α}-dimethyl), B3C, B25H, desB27, B29K(N^{ε}eicosanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(19-carboxynonadecanoylamino)butanoyl]-[CysA10,GluA14,CysB3,HisB25], des-ThrB27,ThrB30-Insulin (human).

LC-MS (electrospray): (m+4)/4: 1506.2 (6020.1)

The following derivatives may be prepared similarly as described above:

### Example 19

### General Procedure (A)

### A1(N^{α},N^{α}-Dimethyl), A10C, A14E, B1(N^{α},N^{α}-dimethyl), B3C, B25H, B29K(N^{ε}eicosanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(19-carboxynonadecanoylamino)butanoyl]-[CysA10,GluA14,CysB3,HisB25], des-ThrB30-Insulin (human).

### Example 20

### General Procedure (A)

### A1(N^{α},N^{α}-Dimethyl), A10C, A14E, B1(N^{α},N^{α}-dimethyl), B3C, B16H, B25H, B29K(N^{ε}eicosanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(19-carboxynonadecanoylamino)butanoyl]-[CysA10,GluA14,CysB3,HisB16,HisB25], des-ThrB30-Insulin (human).

### Example 21

### General Procedure (A)

### A1(N^{α},N^{α}-Dimethyl), A10C, A14E, B1(N^{α},N^{α}-dimethyl), B3C, desB27, B29K(N^{ε}eicosanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(19-carboxynonadecanoylamino)butanoyl]-[CysA10,GluA14,CysB3], des-ThrB27,ThrB30-Insulin (human).

### Example 22

### General Procedure (A)

### A1(N^{α},N^{α}-Dimethyl), A10C, A14E, B1G(N^{α},N^{α}-dimethyl), B3C, B25H, B29K(N^{ε}eicosanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(19-carboxynonadecanoylamino)butanoyl]-[CysA10,GluA14,GlyB1,CysB3,HisB25], des-ThrB30-Insulin (human).

### Example 23

### General Procedure (A)

### A1(N^{α},N^{α}-Dimethyl), A10C, A14E, B1G(N^{α},N^{α}-dimethyl), B3C, B25H, desB27, B29K(N^{ε}eicosanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(19-carboxynonadecanoylamino)butanoyl]-[CysA10,GluA14,GlyB1,CysB3,HisB25], des-ThrB27,ThrB30-Insulin (human).

### Example 24

### General Procedure (A)

### A1(N^{α},N^{α}-Dimethyl), A10C, A14E, B1G(N^{α},N^{α}-dimethyl), B3C, B16H, B25H, B29K(N^{ε}eicosanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(19-carboxynonadecanoylamino)butanoyl]-[CysA10,GluA14,GlyB1,CysB3,HisB16,HisB25], des-ThrB30-Insulin (human).

### Example 25

### General Procedure (A)

### A1(N^{α},N^{α}-Dimethyl), A10C, A14E, B1G(N^{α},N^{α}-dimethyl), B3C, desB27, B29K(N^{ε}eicosanedioyl-gGlu), desB30 Human Insulin

IUPAC (OpenEye, IUPAC style) name:

N{A1},N{A1}-dimethyl, N{B1},N{B1}-dimethyl, N{Epsilon-B29}-[(4S)-4-carboxy-4-(19-carboxynonadecanoylamino)butanoyl]-[CysA10,GluA14,GlyB1,CysB3], des-ThrB27,ThrB30-Insulin (human).

### Example 26

### General Procedure (A)

### A1(N^{α},N^{α}-Dimethyl), A10C, A14E, B1(N^{α},N^{α}-dimethyl), B3C, desB27, B29K(N^{ε}eicosanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[CysA10,GluA14,CysB3], des-ThrB27,ThrB30-Insulin (human)

### Example 27

### General Procedure (A)

### A1(N^{α},N^{α}-Dimethyl), A10C, A14E, B1(N^{α},N^{α}-dimethyl), B3C, B25H, desB27, B29K(N^{ε}eicosanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[CysA10,GluA14,CysB3,B25H], des-ThrB27,ThrB30-Insulin (human)

### Example 28

### General Procedure (A)

### A1(N^{α},N^{α}-Dimethyl), A10C, A14E, B1(N^{α},N^{α}-dimethyl), B3C, B16H, B25H, B29K(N^{ε}eicosanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[CysA10,GluA14,CysB3,B16H,B25H], des-ThrB30-Insulin (human)

### Example 29

### General Procedure (A)

### A1(N^{α},N^{α}-Dimethyl), A10C, A14E, B1G(N^{α},N^{α}-dimethyl), B3C, desB27, B29K(N^{ε}eicosanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[CysA10,GluA14,GlyB1,CysB3], des-ThrB27,ThrB30-Insulin (human)

### Example 30

### General Procedure (A)

### A1(N^{α},N^{α}-Dimethyl), A10C, A14E, B1G(N^{α},N^{α}-dimethyl), B3C, B25H, desB27, B29K(N^{ε}eicosanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[CysA10,GluA14,GlyB1,CysB3,B25H], des-ThrB27,ThrB30-Insulin (human)

### Example 31

### General Procedure (A)

### A1(N^{α},N^{α}-Dimethyl), A10C, A14E, B1G(N^{α},N^{α}-dimethyl), B3C, B25H, B29K(N^{ε}eicosanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[CysA10,GluA14,GlyB1,CysB3,B25H], des-ThrB30-Insulin (human)

### Example 32

### General Procedure (A)

### A1(N^{α},N^{α}-Dimethyl), A10C, A14E, B1 G(N^{α},N^{α}-dimethyl), B3C, B16H, B25H, B29K(N^{ε}eicosanedioyl-gGlu-2×OEG), desB30 Human Insulin

IUPAC (OpenEye, IUPAC style) name:

N{A1},N{A1}-dimethyl, N{B1},N{B1}-dimethyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[CysA10,GluA14,GlyB1,CysB3,B16H,B25H], des-ThrB30-Insulin (human)

General Procedure (B) for Preparation for Carbamoylation of Acylated Insulins of this Invention

The acylated insulin is dissolved in a buffer around physiological pH and an excess of sodium or potassium cyanate is added. The mixture is allowed to stand to completion of the reaction. If necessary, more cyanate is added. The product is isolated by preparative HPLC, ion exchange chromatography, or desalting.

The general procedure (B) is illustrated in the following examples.

### Example 33

### General Procedure (B)

### A1(N^{α}-Carbamoyl), A10C, A14E, B1(N^{α}-carbamoyl), B3C, desB27, B29K(N^{ε}octadecanedioyl-gGlu-2×OEG), desB30 Human Insulin

IUPAC (OpenEye, IUPAC style) name:

N{A1}-carbamoyl, N{B1}-carbamoyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[CysA10,GluA14,CysB3], des-ThrB27,ThrB30-Insulin (human)

A10C, A14E, B3C, desB27, B29K(N^{ε}octadecanedioyl-gGlu-2×OEG), desB30 human insulin (0.18 g) was dissolved in 0.1M sodium phosphate buffer, pH 7.3 (5 mL) and added potassium cyanate (0.12 g). The mixture was stirred gently for 16 h at room temperature. pH was adjusted to 1 with 1N hydrochloric acid and the mixture was purified by preparative HPLC:

### Column: Phenomenex, AXIA, 5, C18, 110, 250×30 cm

Flow: 20 ml/min
Eluent: A: 0.1% TFA in water; B: 0.1% TFA in acetonitrile

### Gradient:

| | | |
|---|---|---|
| 0-7.5 | min: | 25% B |
| 7.5-47.5 | min: | 25% B to 55% B |
| 47.5-52.5 | min: | 55% B |
| 52.5-57.5 | min: | 55% B to 100% B |
| 57.5-60 | min: | 100% B |

Pure fractions were pooled and lyophilised. The product was dissolved in water (50 mL) and pH was adjusted to 8 with 0.1N sodium hydroxide. Lyophilisation afforded the title insulin (70 mg)

LC-MS (electrospray): m/z=1588.32 ((m+4)/4). Calcd: 1588.59

### Example 34

### General Procedure (B)

### A1(N^{α}Carbamoyl), A10C, A14E, B1(N^{α}carbamoyl), B3C, B25H, desB27, B29K(N^{ε}octadecanedioyl-gGlu), desB30 Human Insulin

IUPAC (OpenEye, IUPAC style) name:

### N{A1}-carbamoyl, N{B1}-carbamoyl, N{Epsilon-B29}-[(4S)-4-carboxy-4-(17-carboxyheptadecanoylamino)butanoyl]-[CysA10,GluA14,CysB3,HisB25], des-ThrB27,ThrB30-Insulin (human)

A10C, A14E, B3C, B25H, desB27, B29K(N^{ε}octadecanedioyl-gGlu), desB30 human insulin (300 mg) was dissolved in disodium hydrogen phosphate buffer ph7.5 (2 m mL, 0.1M) and acetonitrile (3 mL). pH was adjusted to 7.5 with 1N sodium hydroxide. Potassium cyanate (204 mg) was added and the mixture was gently stirred at room temperature for 16 hours. The mixture was diluted to 100 mL with water. pH was adjusted to 2.2 with 1N hydrochloric acid and the slightly turbid solution turned clear by addition og acetonitrile (20 mL). The mixture was desalted:

Column: Daiso_{—}200_{—}15um_FEFgel304_ODDMS_{—}30×250 mm
Buffer A: 10% acetonitrile, 0.1% TFA in water
Buffer B: 60% Acetonitrile, 0.1% TFA in water

The mixture was applied to the column and eluted with 1.5 column volumes (CV) buffer A. The derivative was eluted with Buffer B, 1.5 CV, whereby the desired derivative was isolated in the front peak (75 mL).

The mixture was diluted with was water (75 mL) and purified:

### Column: Phenomenex Gemini-NX 5 u C18 110A AXIA P 250×30 mm

A Buffer: 10% Acetonitrile in water+0.1% TFA
B Buffer: 60% Acetonitrile in water+0.1% TFA
Flow: 25 ml/min
Gradient: 20-100% B over 40 min

Pure fractions were pooled and lyophilised. The dry material was dissolved in 40% acetonitrile (30 mL), and pH was adjusted to 8 with 1N sodium hydroxide. Lyophilisation afforded 290 mg of the title compound.

LC-MS (electrospray): m/z: 1513.7 (m+4)/4. Calcd: 1513.5

### Example 35

### General Procedure (B)

### A1(N^{α}-Carbamoyl), A10C, A14E, B1(N^{α}-carbamoyl), B4C, B25H, B29K(N^{ε}eicosanedioyl-gGlu-2×OEG), desB30 Human Insulin

IUPAC (OpenEye, IUPAC style) name:

N{A1}-carbamoyl, N{B1}-carbamoyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[CysA14,CysB4,HisB25], des-ThrB30-Insulin (human)

This insulin was prepared similarly as described above.

LC-MS (electrospray): m/z: 1614.4 (m+4)/4. Calcd: 1614.9

### Example 36

### General Procedure (B)

### A1(N^{α}-Carbamoyl), A10C, A14E, B1(N^{α}-carbamoyl), B3C, desB27, B29K(N^{ε}octadecanedioyl-gGlu), desB30 Human Insulin

IUPAC (OpenEye, IUPAC style) name:

N{A1}-carbamoyl, N{B1}-carbamoyl, N{Epsilon-B29}-[2[2-[2-[[2-[2-[2-[[(4R)-4-carboxy-4-(17-carboxyheptadecanoylamino)butanoyl]amino]ethoxy]ethoxy]acetyl]amino]-ethoxy]ethoxy]acetyl]-[CysA10,GluA14,CysB3], des-ThrB27,ThrB30-Insulin (human)

LC-MS (electrospray): m/z: 1516.05 (m+4)/4. Calcd: 1516.01.

Similarly, the following insulins of the invention may be prepared:

### Example 37

### General Procedure (B)

### A1(N^{α}Carbamoyl), A10C, A14E, B1(N^{α}carbamoyl), B3C, B25H, B29K(N^{ε}octadecanedioyl-gGlu), desB30 Human Insulin

IUPAC (OpenEye, IUPAC style) name:

N{A1}-carbamoyl, N{B1}-carbamoyl, N{Epsilon-B29}-[(4S)-4-carboxy-4-(17-carboxyheptadecanoylamino)butanoyl]-[CysA10,GluA14,CysB3,HisB25], des-ThrB30-Insulin (human)

### Example 38

### General Procedure (B)

### A1(N^{α},N^{α}-Carbamoyl), A10C, A14E, B1(N^{α},N^{α}-carbamoyl), B3C, B16H, B25H, B29K(N^{ε}octadecanedioyl-gGlu), desB30 Human Insulin

IUPAC (OpenEye, IUPAC style) name:

N{A1}-carbamoyl, N{B1}-carbamoyl, N{Epsilon-B29}-[(4S)-4-carboxy-4-(17-carboxyheptadecanoylamino)butanoyl]-[CysA10,GluA14,CysB3,HisB16,HisB25], des-ThrB30-Insulin (human)

### Example 39

### General Procedure (B)

### A1(N^{α}-Carbamoyl), A10C, A14E, B1(N^{α}-carbamoyl), B3C, B25H, desB27, B29K(N^{ε}octadecanedioyl-gGlu-2×OEG), desB30 Human Insulin

IUPAC (OpenEye, IUPAC style) name:

N{A1}-carbamoyl, N{B1}-carbamoyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[CysA10,GluA14,CysB3,HisB25], des-ThrB27,ThrB30-Insulin (human)

### Example 40

### General Procedure (B)

### A1(N^{α}-Carbamoyl), A10C, A14E, B1(N^{α}-carbamoyl), B3C, B25H, B29K(N^{ε}octadecanedioyl-gGlu-2×OEG), desB30 Human Insulin

IUPAC (OpenEye, IUPAC style) name:

N{A1}-carbamoyl, N{B1}-carbamoyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[CysA10,GluA14,CysB3,HisB25], des-ThrB30-Insulin (human)

### Example 41

### General Procedure (B)

### A1(N^{α}-Carbamoyl), A10C, A14E, B1(N^{α}-carbamoyl), B3C, B16H, B25H, B29K(N^{ε}octadecanedioyl-gGlu-2×OEG), desB30 Human Insulin

IUPAC (OpenEye, IUPAC style) name:

N{A1}-carbamoyl, N{B1}-carbamoyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]-ethoxy]ethoxy]acetyl]-[CysA10,GluA14,CysB3,HisB16,HisB25], des-ThrB30-Insulin (human)

### Example 42

### General Procedure (B)

### A1(N^{α}-Carbamoyl), A10C, A14E, B1(N^{α}-carbamoyl), B3C, B25H, desB27, B29K(N^{ε}eicosanedioyl-gGlu), desB30 Human Insulin

IUPAC (OpenEye, IUPAC style) name:

N{A1}-carbamoyl, N{B1}-carbamoyl, N{Epsilon-B29}-[(4S)-4-carboxy-4-(19-carboxynonadecanoylamino)butanoyl]-[CysA10,GluA14,CysB3,HisB25], des-ThrB27,ThrB30-Insulin (human)

### Example 43

### General Procedure (B)

### A1(N^{α}-Carbamoyl), A10C, A14E, B1(N^{α}-carbamoyl), B3C, desB27, B29K(N^{ε}eicosanedioyl-gGlu), desB30 Human Insulin

IUPAC (OpenEye, IUPAC style) name:

N{A1}-carbamoyl, N{B1}-carbamoyl, N{Epsilon-B29}-[(4S)-4-carboxy-4-(19-carboxy-nonadecanoylamino)butanoyl]-[CysA10,GluA14,CysB3], des-ThrB27,ThrB30-Insulin (human)

### Example 44

### General Procedure (B)

### A1(N^{α}-Carbamoyl), A10C, A14E, B1(N^{α}-carbamoyl), B3C, B25H, B29K(N^{ε}eicosanedioyl-gGlu), desB30 Human Insulin

IUPAC (OpenEye, IUPAC style) name:

N{A1}-carbamoyl, N{B1}-carbamoyl, N{Epsilon-B29}-[(4S)-4-carboxy-4-(19-carboxynonadecanoylamino)butanoyl]-[CysA10,GluA14,CysB3,HisB25], des-ThrB30-Insulin (human)

### Example 45

### General Procedure (B)

### A1(N^{α}-Carbamoyl), A10C, A14E, B1(N^{α}-carbamoyl), B3C, B16H, B25H, B29K(N^{ε}eicosanedioyl-gGlu), desB30 Human Insulin

IUPAC (OpenEye, IUPAC style) name:

N{A1}-carbamoyl, N{B1}-carbamoyl, N{Epsilon-B29}-[(4S)-4-carboxy-4-(19-carboxynonadecanoylamino)butanoyl]-[CysA10,GluA14,CysB3,HisB16,HisB25], des-ThrB30-Insulin (human)

### Example 46

### General Procedure (B)

### A1(N^{α}-Carbamoyl), A10C, A14E, B1(N^{α}-carbamoyl), B3C, B25H, desB27, B29K(N^{ε}eicosanedioyl-gGlu-2×OEG), desB30 Human Insulin

IUPAC (OpenEye, IUPAC style) name:

N{A1}-carbamoyl, N{B1}-carbamoyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[CysA10,GluA14,CysB3,HisB25], des-ThrB27,ThrB30-Insulin (human)

### Example 47

### General Procedure (B)

### A1(N^{α}-Carbamoyl), A10C, A14E, B1(N^{α}-carbamoyl), B3C, desB27, B29K(N^{ε}eicosanedioyl-gGlu-2×OEG), desB30 human insulin

IUPAC (OpenEye, IUPAC style) name:

N{A1}-carbamoyl, N{B1}-carbamoyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]-ethoxy]ethoxy]acetyl]-[CysA10,GluA14,CysB3], des-ThrB27,ThrB30-Insulin (human)

### Example 48

### General Procedure (B)

### A1(N^{α}-Carbamoyl), A10C, A14E, B1(N^{α}-carbamoyl), B3C, B25H, B29K(N^{ε}eicosanedioyl-gGlu-2×OEG), desB30 Human Insulin

IUPAC (OpenEye, IUPAC style) name:

N{A1}-carbamoyl, N{B1}-carbamoyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl][CysA10,GluA14,CysB3,HisB25], des-ThrB30-Insulin (human)

### Example 49

### General Procedure (B)

A1(N^{α}-Carbamoyl), A10C, A14E, B1(N^{α}-carbamoyl), B3C, B16H, B25H, B29K(N^{ε}eicosanedioyl-gGlu-2×OEG), desB30 Human Insulin

IUPAC (OpenEye, IUPAC style) name:

N{A1}-carbamoyl, N{B1}-carbamoyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[CysA10,GluA14,CysB3,HisB16,HisB25], des-ThrB30-Insulin (human)

General Procedure (C) for Preparation for N-Terminal Acylation of Acylated Insulins of this Invention

The lysine-acylated insulin is dissolved in a buffer, optionally containing an organic co-solvent. pH of the mixture may be from neutral to alkaline (e.g from around 6-8—depending on the solubility of the insulin in question—up to 13 or 14) and an excess of acylation reagent, eg. as N-hydroxysuccinimide ester (OSu), is added. The mixture is allowed to stand to completion of the reaction. If necessary, more acylation reagent is added. The product is isolated by preparative HPLC.

Alternatively, the reaction may be performed under anhydrous conditions, eg in DMSO containing an organic base, e.g. triethylamine.

The following insulins of the invention may be prepared according to this general procedure (C).

### Example 50

### General Procedure (C)

### A1(N^{α}-Acetyl), A10C, A14E, B1(N^{α}-acetyl), B3C, B25H, desB27, B29K(N^{ε}octadecanedioyl-gGlu), desB30 Human Insulin

IUPAC (OpenEye, IUPAC style) name:

N{A1}-acetyl, N{B1}-acetyl, N{Epsilon-B29}-[(4S)-4-carboxy-4-(17-carboxyheptadecanoylamino)butanoyl]-[CysA10,GluA14,CysB3,HisB25], des-ThrB27,ThrB30-Insulin (human)

### Example 51

### General Procedure (C)

### A1(N^{α}-Acetyl), A10C, A14E, B1(N^{α}-acetyl), B3C, desB27, B29K(N^{ε}octadecanedioyl-gGlu-2×OEG), desB30 Human Insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[CysA10,GluA14,CysB3], des-ThrB27,ThrB30-Insulin (human)

### Example 52

### General Procedure (C)

### A1(N^{α}-Acetyl), A10C, A14E, B1(N^{α}-acetyl), B3C, desB27, B29K(N^{ε}octadecanedioyl-gGlu), desB30 Human Insulin

IUPAC (OpenEye, IUPAC style) name:

N{A1}-acetyl, N{B1}-acetyl, N{Epsilon-B29}-[2-[2-[2-[[2-[2-[2-[[(4R)-4-carboxy-4-(17-carboxyheptadecanoylamino)butanoyl]amino]ethoxy]ethoxy]acetyl]amino]ethoxy]-ethoxy]acetyl]-[GluA14,HisB25], des-ThrB27,ThrB30-Insulin (human)

### Example 53

### General Procedure (C)

### A1(N^{α}-Acetyl), A10C, A14E, B1(N^{α}-acetyl), B3C, B25H, B29K(N^{ε}octadecanedioyl-gGlu), desB30 Human Insulin

IUPAC (OpenEye, IUPAC style) name:

N{A1}-acetyl, N{B1}-acetyl, N{Epsilon-B29}-[(4S)-4-carboxy-4-(17-carboxyheptadecanoylamino)butanoyl]-[CysA10,GluA14,CysB3,HisB25], des-ThrB30-Insulin (human)

### Example 54

### General Procedure (C)

### A1(N^{α}-Acetyl), A10C, A14E, B1(N^{α}-acetyl), B3C, B16H, B25H, B29K(N^{ε}octadecanedioyl-gGlu), desB30 Human Insulin

IUPAC (OpenEye, IUPAC style) name:

N{A1}-acetyl, N{B1}-acetyl, N{Epsilon-B29}-[(4S)-4-carboxy-4-(17-carboxyheptadecanoylamino)butanoyl]-[CysA10,GluA14,CysB3,HisB16,HisB25], des-ThrB30-Insulin (human)

### Example 55

### General Procedure (C)

### A1(N^{α}-Acetyl), A10C, A14E, B1(N^{α}-acetyl), B3C, B25H, desB27, B29K(N^{ε}octadecanedioyl-gGlu-2×OEG), desB30 Human Insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[CysA10,GluA14,CysB3,HisB25], des-ThrB27,ThrB30-Insulin (human)

### Example 56

### General Procedure (C)

### A1(N^{α}-Acetyl), A10C, A14E, B1(N^{α}-acetyl), B3C, B25H, B29K(N^{ε}octadecanedioyl-gGlu-2×OEG), desB30 Human Insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[CysA10,GluA14,CysB3,HisB25], des-ThrB30-Insulin (human)

### Example 57

### General Procedure (C)

### A1(N^{α}-Acetyl), A10C, A14E, B1(N^{α}-acetyl), B3C, B16H, B25H, B29K(N^{ε}octadecanedioyl-gGlu-2×OEG), desB30 Human Insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[CysA10,GluA14,CysB3,HisB16,HisB25], des-ThrB30-Insulin (human)

### Example 58

### General Procedure (C)

### A1(N^{α}-Acetyl), A10C, A14E, B1(N^{α}-acetyl), B3C, B25H, desB27, B29K(N^{ε}eicosanedioyl-gGlu), desB30 Human Insulin

IUPAC (OpenEye, IUPAC style) name:

N{A1}-acetyl, N{B1}-acetyl, N{Epsilon-B29}-[(4S)-4-carboxy-4-(19-carboxynonadecanoylamino)butanoyl]-[CysA10,GluA14,CysB3,HisB25], des-ThrB27,ThrB30-Insulin (human)

### Example 59

### General Procedure (C)

### A1(N^{α}-Acetyl), A10C, A14E, B1(N^{α}-acetyl), B3C, desB27, B29K(N^{ε}eicosanedioyl-gGlu), desB30 Human Insulin

IUPAC (OpenEye, IUPAC style) name:

N{A1}-acetyl, N{B1}-acetyl, N{Epsilon-B29}-[(4S)-4-carboxy-4-(19-carboxynonadecanoylamino)butanoyl]-[CysA10,GluA14,CysB3], des-ThrB27,ThrB30-Insulin (human)

### Example 60

### General Procedure (C)

### A1(N^{α}-Acetyl), A10C, A14E, B1(N^{α}-acetyl), B3C, B25H, B29K(N^{ε}eicosanedioyl-gGlu), desB30 Human Insulin

IUPAC (OpenEye, IUPAC style) name:

N{A1}-acetyl, N{B1}-acetyl, N{Epsilon-B29}-[(4S)-4-carboxy-4-(19-carboxynonadecanoylamino)butanoyl]-[CysA10,GluA14,CysB3,HisB25], des-ThrB30-Insulin (human)

### Example 61

### General Procedure (C)

A1(N^{α}-Acetyl), A10C, A14E, B1(N^{α}-acetyl), B3C, B16H, B25H, B29K(N^{ε}eicosanedioyl-gGlu), desB30 Human Insulin

IUPAC (OpenEye, IUPAC style) name:

N{A1}-acetyl, N{B1}-acetyl, N{Epsilon-B29}-[(4S)-4-carboxy-4-(19-carboxynonadecanoylamino)butanoyl]-[CysA10,GluA14,CysB3,HisB16,HisB25], des-ThrB30-Insulin (human)

### Example 62

### General Procedure (C)

### A1(N^{α}-Acetyl), A10C, A14E, B1(N^{α}-acetyl), B3C, B25H, desB27, B29K(N^{ε}eicosanedioyl-gGlu-2×OEG), desB30 Human Insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[CysA10,GluA14,CysB3,HisB25], des-ThrB27,ThrB30-Insulin (human)

### Example 63

### General Procedure (C)

### A1(N^{α}-Acetyl), A10C, A14E, B1(N^{α}-acetyl), B3C, desB27, B29K(N^{ε}eicosanedioyl-gGlu-2×OEG), desB30 Human Insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[CysA10,GluA14,CysB3], des-ThrB27,ThrB30-Insulin (human)

### Example 64

### General Procedure (C)

### A1(N^{α}-Acetyl), A10C, A14E, B1(N^{α}-acetyl), B3C, B25H, B29K(N^{ε}eicosanedioyl-gGlu-2×OEG), desB30 Human Insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[CysA10,GluA14,CysB3,HisB25], des-ThrB30-Insulin (human)

### Example 65

### General Procedure (C)

### A1(N^{α}-Acetyl), A10C, A14E, B1(N^{α}-acetyl), B3C, B16H, B25H, B29K(N^{ε}eicosanedioyl-gGlu-2×OEG), desB30 Human Insulin

IUPAC (OpenEye, IUPAC style) name:

N{A1}-acetyl, N{B1}-acetyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[CysA10,GluA14,CysB3,HisB16,HisB25], des-ThrB30-Insulin (human)

General Procedure (D) for Preparation for N-Terminal Acylation of Acylated Insulins of this Invention Using (Cyclic) Carboxylic Acid Anhydrides

The lysine-acylated insulin is dissolved in a polar aprotic solvent, optionally containing an organic base, such as triethyl amine or N,N-diisopropylethylamine and an excess of acylation reagent, eg. as succinic, glutaric or diglycolic acid anhydride is added. The mixture is allowed to stand to completion of the reaction. If necessary, more acylation reagent is added. The product is isolated, eg. by preparative HPLC or by anion exchange chromatography.

Alternatively, Procedure (D) can be performed in an aqueous media using N-hydroxysuccinimide activated diacids (or anhydrides) as illustrated in example 55.

The following derivatives may be prepared as described in the general procedure (D):

### Example 66

### General Procedure (D)

### A1(N^{α}-Glutaryl), A10C, A14E, B1(N^{α}-glutaryl), B3C, B25H, desB27, B29K(N^{ε}octadecanedioyl-gGlu), desB30 Human Insulin

IUPAC (OpenEye, IUPAC style) name:

N{A1}-glutaryl, N{B1}-glutaryl, N{Epsilon-B29}-[(4S)-4-carboxy-4-(17-carboxyheptadecanoylamino)butanoyl]-[CysA10,GluA14,CysB3,HisB25], des-ThrB27,ThrB30-Insulin (human)

### Example 67

### General Procedure (D)

### A1(N^{α}-Glutaryl), A10C, A14E, B1(N^{α}-glutaryl), B3C, desB27, B29K(N^{ε}octadecanedioyl-gGlu-2×OEG), desB30 Human Insulin

IUPAC (OpenEye, IUPAC style) name:

N{A1}-glutaryl, N{B1}-glutaryl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[CysA10,GluA14,CysB3], des-ThrB27,ThrB30-Insulin (human)

### Example 68

### General Procedure (D)

### A1(N^{α}-Glutaryl), A10C, A14E, B1(N^{α}-glutaryl), B3C, desB27, B29K(N^{ε}octadecanedioyl-gGlu), desB30 Human Insulin

IUPAC (OpenEye, IUPAC style) name:

N{A1}-glutaryl, N{B1}-glutaryl, N{Epsilon-B29}-[2-[2-[2-[[2-[2-[2-[[(4R)-4-carboxy-4-(17-carboxyheptadecanoylamino)butanoyl]amino]ethoxy]ethoxy]acetyl]amino]ethoxy]-ethoxy]acetyl]-[GluA14,HisB25], des-ThrB27,ThrB30-Insulin (human)

### Example 69

### General Procedure (D)

### A1(N^{α}-Glutaryl), A10C, A14E, B1(N^{α}-glutaryl), B3C, B25H, B29K(N^{ε}octadecanedioyl-gGlu), desB30 Human Insulin

IUPAC (OpenEye, IUPAC style) name:

N{A1}-glutaryl, N{B1}-glutaryl, N{Epsilon-B29}-[(4S)-4-carboxy-4-(17-carboxyheptadecanoylamino)butanoyl]-[CysA10,GluA14,CysB3,HisB25], des-ThrB30-Insulin (human)

### Example 70

### General Procedure (D)

### A1(N^{α}-Glutaryl), A10C, A14E, B1(N^{α}-glutaryl), B3C, B16H, B25H, B29K(N^{ε}octadecanedioyl-gGlu), desB30 Human Insulin

IUPAC (OpenEye, IUPAC style) name:

N{A1}-glutaryl, N{B1}-glutaryl, N{Epsilon-B29}-[(4S)-4-carboxy-4-(17-carboxyheptadecanoylamino)butanoyl]-[CysA10,GluA14,CysB3,HisB16,HisB25], des-ThrB30-Insulin (human)

### Example 71

### General Procedure (D)

### A1(N^{α}-Glutaryl), A10C, A14E, B1(N^{α}-glutaryl), B3C, B25H, desB27, B29K(N^{ε}octadecanedioyl-gGlu-2×OEG), desB30 Human Insulin

IUPAC (OpenEye, IUPAC style) name:

N{A1}-glutaryl, N{B1}-glutaryl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[CysA10,GluA14,CysB3,HisB25], des-ThrB27,ThrB30-Insulin (human)

### Example 72

### General Procedure (D)

### A1(N^{α}-Glutaryl), A10C, A14E, B1(N^{α}-glutaryl), B3C, B25H, B29K(N^{ε}octadecanedioyl-gGlu-2×OEG), desB30 Human Insulin

IUPAC (OpenEye, IUPAC style) name:

N{A1}-glutaryl, N{B1}-glutaryl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[CysA10,GluA14,CysB3,HisB25], des-ThrB30-Insulin (human)

### Example 73

### General Procedure (D)

### A1(N^{α}-Glutaryl), A10C, A14E, B1(N^{α}-glutaryl), B3C, B16H, B25H, B29K(N^{ε}octadecanedioyl-gGlu-2×OEG), desB30 Human Insulin

IUPAC (OpenEye, IUPAC style) name:

N{A1}-glutaryl, N{B1}-glutaryl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[CysA10,GluA14,CysB3,HisB16,HisB25], des-ThrB30-Insulin (human)

### Example 74

### General Procedure (D)

### A1(N^{α}-Glutaryl), A10C, A14E, B1(N^{α}-glutaryl), B3C, B25H, desB27, B29K(N^{ε}eicosanedioyl-gGlu), desB30 Human Insulin

IUPAC (OpenEye, IUPAC style) name:

N{A1}-glutaryl, N{B1}-glutaryl, N{Epsilon-B29}-[(4S)-4-carboxy-4-(19-carboxynonadecanoylamino)butanoyl]-[CysA10,GluA14,CysB3,HisB25], des-ThrB27,ThrB30-Insulin (human)

### Example 75

### General Procedure (D)

### A1(N^{α}-Glutaryl), A10C, A14E, B1(N^{α}-glutaryl), B3C, desB27, B29K(N^{ε}eicosanedioyl-gGlu), desB30 Human Insulin

IUPAC (OpenEye, IUPAC style) name:

N{A1}-glutaryl, N{B1}-glutaryl, N{Epsilon-B29}-[(4S)-4-carboxy-4-(19-carboxynonadecanoylamino)butanoyl]-[CysA10,GluA14,CysB3], des-ThrB27,ThrB30-Insulin (human)

### Example 76

### General Procedure (D)

### A1(N^{α}-Glutaryl), A10C, A14E, B1(N^{α}-glutaryl), B3C, B25H, B29K(N^{ε}eicosanedioyl-gGlu), desB30 Human Insulin

IUPAC (OpenEye, IUPAC style) name:

N{A1}-glutaryl, N{B1}-glutaryl, N{Epsilon-B29}-[(4S)-4-carboxy-4-(19-carboxynonadecanoylamino)butanoyl]-[CysA10,GluA14,CysB3,HisB25], des-ThrB30-Insulin (human)

### Example 77

### General Procedure (D)

### A1(N^{α}-Glutaryl), A10C, A14E, B1(N^{α}-glutaryl), B3C, B16H, B25H, B29K(N^{ε}eicosanedioyl-gGlu), desB30 Human Insulin

IUPAC (OpenEye, IUPAC style) name:

N{A1}-glutaryl, N{B1}-glutaryl, N{Epsilon-B29}-[(4S)-4-carboxy-4-(19-carboxynonadecanoylamino)butanoyl]-[CysA10,GluA14,CysB3,HisB16,HisB25], des-ThrB30-Insulin (human)

### Example 78

### General Procedure (D)

### A1(N^{α}-Glutaryl), A10C, A14E, B1(N^{α}-glutaryl), B3C, B25H, desB27, B29K(N^{ε}eicosanedioyl-gGlu-2×OEG), desB30 Human Insulin

IUPAC (OpenEye, IUPAC style) name:

N{A1}-glutaryl, N{B1}-glutaryl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[CysA10,GluA14,CysB3,HisB25], des-ThrB27,ThrB30-Insulin (human)

### Example 79

### General Procedure (D)

### A1(N^{α}-Glutaryl), A10C, A14E, B1(N^{α}-glutaryl), B3C, desB27, B29K(N^{ε}eicosanedioyl-gGlu-2×OEG), desB30 Human Insulin

IUPAC (OpenEye, IUPAC style) name:

N{A1}-glutaryl, N{B1}-glutaryl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl][CysA10,GluA14,CysB3], des-ThrB27,ThrB30-Insulin (human)

### Example 80

### General Procedure (D)

### A1(N^{α}-Glutaryl), A10C, A14E, B1(N^{α}-glutaryl), B3C, B25H, B29K(N^{ε}eicosanedioyl-gGlu-2×OEG), desB30 Human Insulin

IUPAC (OpenEye, IUPAC style) name:

N{A1}-glutaryl, N{B1}-glutaryl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[CysA10,GluA14,CysB3,HisB25], des-ThrB30-Insulin (human)

### Example 81

### General Procedure (D)

### A1(N^{α}-Glutaryl), A10C, A14E, B1(N^{α}-glutaryl), B3C, B16H, B25H, B29K(N^{ε}eicosanedioyl-gGlu-2×OEG), desB30 Human Insulin

IUPAC (OpenEye, IUPAC style) name:

N{A1}-glutaryl, N{B1}-glutaryl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[CysA10,GluA14,CysB3,HisB16,HisB25], des-ThrB30-Insulin (human)

### Example 82

### General Procedure (D)

### A1(N^{α}-Diglycolyl), A10C, A14E, B1(N^{α}-diglycolyl), B3C, B25H, desB27, B29K(N^{ε}octadecanedioyl-gGlu), desB30 Human Insulin

IUPAC (OpenEye, IUPAC style) name:

N{A1}-diglycolyl, N{B1}-diglycolyl, N{Epsilon-B29}-[(4S)-4-carboxy-4-(17-carboxyheptadecanoylamino)butanoyl]-[CysA10,GluA14,CysB3,HisB25], des-ThrB27,ThrB30-Insulin (human)

### Example 83

### General Procedure (D)

### A1(N^{α}-Diglycolyl), A10C, A14E, B1(N^{α}-diglycolyl), B3C, desB27, B29K(N^{ε}octadecanedioyl-gGlu-2×OEG), desB30 Human Insulin

N{A1}-diglycolyl, N{B1}-diglycolyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[CysA10,GluA14,CysB3], des-ThrB27,ThrB30-Insulin (human)

### Example 84

### General Procedure (D)

### A1(N^{α}-Diglycolyl), A10C, A14E, B1(N^{α}-diglycolyl), B3C, desB27, B29K(N octadecanedioyl-gGlu), desB30 Human Insulin

IUPAC (OpenEye, IUPAC style) name:

N{A1}-diglycolyl, N{B1}-diglycolyl, N{Epsilon-B29}-[2-[2-[2-[[2-[2-[2-[[(4R)-4-carboxy-4-(17-carboxyheptadecanoylamino)butanoyl]amino]ethoxy]ethoxy]acetyl]amino]ethoxy]-ethoxy]acetyl]-[GluA14,HisB25], des-ThrB27,ThrB30-Insulin (human)

### Example 85

### General Procedure (D)

### A1(N^{α}-Diglycolyl), A10C, A14E, B1(N^{α}-diglycolyl), B3C, B25H, B29K(N^{ε}octadecanedioyl-gGlu), desB30 Human Insulin

IUPAC (OpenEye, IUPAC style) name:

N{A1}-diglycolyl, N{B1}-diglycolyl, N{Epsilon-B29}-[(4S)-4-carboxy-4-(17-carboxyheptadecanoylamino)butanoyl]-[CysA10,GluA14,CysB3,HisB25], des-ThrB30-Insulin (human)

### Example 86

### General Procedure (D)

### A1(N^{α}-Diglycolyl), A10C, A14E, B1(N^{α}-diglycolyl), B3C, B16H, B25H, B29K(N^{ε}octadecanedioyl-gGlu), desB30 Human Insulin

IUPAC (OpenEye, IUPAC style) name:

N{A1}-diglycolyl, N{B1}-diglycolyl, N{Epsilon-B29}-[(4S)-4-carboxy-4-(17-carboxyheptadecanoylamino)butanoyl]-[CysA10,GluA14,CysB3,HisB16,HisB25], des-ThrB30-Insulin (human)

### Example 87

### General Procedure (D)

### A1(N^{α}-Diglycolyl), A10C, A14E, B1(N^{α}-diglycolyl), B3C, B25H, desB27, B29K(N^{ε}octadecanedioyl-gGlu-2×OEG), desB30 Human Insulin

IUPAC (OpenEye, IUPAC style) name:

N{A1}-diglycolyl, N{B1}-diglycolyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[CysA10,GluA14,CysB3,HisB25], des-ThrB27,ThrB30-Insulin (human)

### Example 88

### General Procedure (D)

### A1(N^{α}-Diglycolyl), A10C, A14E, B1(N^{α}-diglycolyl), B3C, B25H, B29K(N^{ε}octadecanedioyl-gGlu-2×OEG), desB30 Human Insulin

IUPAC (OpenEye, IUPAC style) name:

N{A1}-diglycolyl, N{B1}-diglycolyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[CysA10,GluA14,CysB3,HisB25], des-ThrB30-Insulin (human)

### Example 89

### General Procedure (D)

### A1(N^{α}-Diglycolyl), A10C, A14E, B1(N^{α}-diglycolyl), B3C, B16H, B25H, B29K(N^{ε}octadecanedioyl-gGlu-2×OEG), desB30 Human Insulin

IUPAC (OpenEye, IUPAC style) name:

N{A1}-diglycolyl, N{B1}-diglycolyl, N{Epsilon-B29}-[2-[2-[2-[[2-[2-[2-[[(4S)-4-carboxy-4-(17-carboxyheptadecanoylamino)butanoyl]amino]ethoxy]ethoxy]acetyl]amino]ethoxy]-ethoxy]acetyl]-[CysA10,GluA14,CysB3,HisB16,HisB25], des-ThrB30-Insulin (human)

### Example 90

### General Procedure (D)

### A1(N^{α}-Diglycolyl), A10C, A14E, B1(N^{α}-diglycolyl), B3C, B25H, desB27, B29K(N^{ε}eicosanedioyl-gGlu), desB30 Human Insulin

IUPAC (OpenEye, IUPAC style) name:

N{A1}-diglycolyl, N{B1}-diglycolyl, N{Epsilon-B29}-[(4S)-4-carboxy-4-(19-carboxynonadecanoylamino)butanoyl]-[CysA10,GluA14,CysB3,HisB25], des-ThrB27,ThrB30-Insulin (human)

### Example 91

### General Procedure (D)

### A1(N^{α}-Diglycolyl), A10C, A14E, B1(N^{α}-diglycolyl), B3C, desB27, B29K(N eicosanedioyl-gGlu), desB30 Human Insulin

IUPAC (OpenEye, IUPAC style) name:

N{A1}-diglycolyl, N{B1}-diglycolyl, N{Epsilon-B29}-[(4S)-4-carboxy-4-(19-carboxynonadecanoylamino)butanoyl]-[CysA10,GluA14,CysB3], des-ThrB27,ThrB30-Insulin (human)

### Example 92

### General Procedure (D)

### A1(N^{α}-Diglycolyl), A10C, A14E, B1(N^{α}-diglycolyl), B3C, B25H, B29K(N^{ε}eicosanedioyl-gGlu), desB30 Human Insulin

IUPAC (OpenEye, IUPAC style) name:

N{A1}-diglycolyl, N{B1}-diglycolyl, N{Epsilon-B29}-[(4S)-4-carboxy-4-(19-carboxynonadecanoylamino)butanoyl]-[CysA10,GluA14,CysB3,HisB25], des-ThrB30-Insulin (human)

### Example 93

### General Procedure (D)

### A1(N^{α}-Diglycolyl), A10C, A14E, B1(N^{α}-diglycolyl), B3C, B16H, B25H, B29K(N^{ε}eicosanedioyl-gGlu), desB30 Human Insulin

IUPAC (OpenEye, IUPAC style) name:

N{A1}-diglycolyl, N{B1}-diglycolyl, N{Epsilon-B29}-[(4S)-4-carboxy-4-(19-carboxynonadecanoylamino)butanoyl]-[CysA10,GluA14,CysB3,HisB16,HisB25], des-ThrB30-Insulin (human)

### Example 94

### General Procedure (D)

### A1(N^{α}-Diglycolyl), A10C, A14E, B1(N^{α}-diglycolyl), B3C, B25H, desB27, B29K(N^{ε}eicosanedioyl-gGlu-2×OEG), desB30 Human Insulin

IUPAC (OpenEye, IUPAC style) name:

N{A1}-diglycolyl, N{B1}-diglycolyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[CysA10,GluA14,CysB3,HisB25], des-ThrB27,ThrB30-Insulin (human)

### Example 95

### General Procedure (D)

### A1(N^{α}-Diglycolyl), A10C, A14E, B1(N^{α}-diglycolyl), B3C, desB27, B29K(N^{ε}eicosanedioyl-gGlu-2×OEG), desB30 Human Insulin

IUPAC (OpenEye, IUPAC style) name:

N{A1}-diglycolyl, N{B1}-diglycolyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[CysA10,GluA14,CysB3], des-ThrB27,ThrB30-Insulin (human)

### Example 96

### General Procedure (D)

### A1(N^{α}-Diglycolyl), A10C, A14E, B1(N^{α}-diglycolyl), B3C, B25H, B29K(N^{ε}eicosanedioyl-gGlu-2×OEG), desB30 Human Insulin

IUPAC (OpenEye, IUPAC style) name:

N{A1}-diglycolyl, N{B1}-diglycolyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[CysA10,GluA14,CysB3,HisB25], des-ThrB30-Insulin (human)

### Example 97

### General Procedure (D)

### A1(N^{α}-Diglycolyl), A10C, A14E, B1(N^{α}-diglycolyl), B3C, B16H, B25H, B29K(N eicosanedioyl-gGlu-2×OEG), desB30 Human Insulin

N{A1}-diglycolyl, N{B1}-diglycolyl, N{Epsilon-B29}-[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]-acetyl]-[CysA10,GluA14,CysB3,HisB16,HisB25], des-ThrB30-Insulin (human)

### Example 98

### Insulin Receptor Affinity of Selected N-Terminally Modified Insulins of the Invention

The affinity of the acylated insulin analogues of this invention for the human insulin receptor is determined by a SPA assay (Scintillation Proximity Assay) microtiterplate antibody capture assay. SPA-PVT antibody-binding beads, anti-mouse reagent (Amersham Bio-sciences, Cat No. PRNQ0017) are mixed with 25 ml of binding buffer (100 mM HEPES pH 7.8; 100 mM sodium chloride, 10 mM MgSO₄, 0.025% Tween-20). Reagent mix for a single Packard Optiplate (Packard No. 6005190) is composed of 2.4 μl of a 1:5000 diluted purified recombinant human insulin receptor (either with or without exon 11), an amount of a stock solution of A14Tyr[¹²⁵I]-human insulin corresponding to 5000 cpm per 100 μl of reagent mix, 12 μl of a 1:1000 dilution of F12 antibody, 3 ml of SPA-beads and binding buffer to a total of 12 ml. A total of 100 μl reagent mix is then added to each well in the Packard Optiplate and a dilution series of the N-terminally modified insulin is made in the Optiplate from appropriate samples. The samples are then incubated for 16 hours while gently shaken. The phases are the then separated by centrifugation for 1 min and the plates counted in a Topcounter. The binding data were fitted using the nonlinear regression algorithm in the GraphPad Prism 2.01 (Graph Pad Software, San Diego, Calif.) and affinities are expressed relative (in percentage (%)) to the affinity of human insulin.

A related assay is also used wherein the binding buffer also contains 1.5% HSA in order to mimic physiological conditions

| | | | Lipogenesis | |
|---|---|---|---|---|
| | Relative | Relative | in rat | Hydro- |
| | IR-A | IR-A | adipocytres | phobicity |
| | affinity | affinity | (@ 0.1% HSA) | rel. to |
| Example | (@ 0% HSA) | (@ 1.5% HSA) | rel. to human | human |
| No. | (%) | (%)e | insulin (%) | insulin |
| Prior art′ | 2.3 | 0.11 | 0.31 | 0.31 |
| 1 | 0.72 | 0.02 | | 0.12 |
| 2 | 0.4 | 0.03 | 0.025 | 0.38 |
| 3 | | 0.05 | | 0.065 |
| 11 | 0.6 | 0.04 | | 0.095 |
| 12 | 0.4 | 0.04 | | 0.077 |
| 28 | 0.1 | 0.01 | | 0.298 |
| 34 | 0.1 | 0.00 | | |
| 33 | 0.6 | | | |
| 36 | 0.7 | 0.01 | | |
| 46 | 0.1 | 0.01 | | 0.132 |
| 67 | 0.4 | 0.17 | | 0.0156 |
| 83 | 0.4 | | | 0.0169 |
| 18 | 0.72 | 0.02 | | 0.12 |

*) Insulin of example 1 without N-terminal modification

### Example 99

### Hydrophobicity of the N-Terminally Modified Insulins of the Invention

The hydrophobicity of an N-terminally modified insulin is found by reverse phase HPLC run under isocratic conditions. The elution time of the N-terminally modified insulin is compared to that of human insulin (herein designated HI) or another derivative with a known hydrophibicity under the same conditions. The hydrophobicity, k′rel, is calculated as: k′rel_{deriv}=((t_{deriv}−t₀)(t_{ref}−t₀)*k′rel_{ref}. Using HI as reference: k′rel_{ref}=k′rel_{HI}=1. The void time of the HPLC system, t₀, is determined by injecting 5 μl of 0.1 mM NaNO₃. Running conditions:

Column: Lichrosorb RP-C18, 5 μm, 4×250 mm

Buffer A: 0.1 M natrium phosphate pH 7.3, 10 vol % CH₃CN

Buffer B: 50 vol % CH₃CN

Injection volume: 5 μl

Run time: max 60 minutes

After running an initial gradient, the isocratic level for running the derivative and reference (for example HI) is chosen, and the elution times of the derivative and reference under isocratic conditions are used in the above equation to calculate k′rel_{deriv}.

### Example 100

### Degradation of Insulin Analogs Using Duodenum Lumen Enzymes

Degradation of insulin analogs using duodenum lumen enzymes (prepared by filtration of duodenum lumen content) from SPD rats. The assay is performed by a robot in a 96 well plate (2 ml) with 16 wells available for insulin analogs and standards. Insulin analogs ˜15 μM are incubated with duodenum enzymes in 100 mM Hepes, pH=7.4 at 37° C., samples are taken after 1, 15, 30, 60, 120 and 240 min and reaction quenched by addition of TFA. Intact insulin analogs at each point are determined by RP-HPLC. Degradation half time is determined by exponential fitting of the data and normalized to half time determined for the reference insulins, A14E, B25H, desB30 human insulin or human insulin in each assay. The amount of enzymes added for the degradation is such that the half time for degradation of the reference insulin is between 60 min and 180 min. The result is given as the degradation half time for the insulin analog in rat duodenum divided by the degradation half time of the reference insulin from the same experiment (relative degradation rate).

| | Stability towards duodenum |
|---|---|
| | lumen enzymes (fold relative to |
| Example No. | A14E, B25H, desB30 human insulin)) |
| Prior art′ | |
| 2 | 1.5 |
| 3 | 4.5 |
| 12 | 3.2 |
| 28 | 1.6 |
| 34 | 1.8 |
| 33 | 1.6 |
| 46 | 1.2 |
| 83 | 2.0 |

### Example 101

### Lipogenesis in Rat Adipocytes

As a measure of in vitro potency of the insulins of the invention, lipogenesis can be used.

Primary rat adipocytes are isolated from the epididymale fat pads and incubated with 3H-glucose in buffer containing e.g. 0.1% fat free HSA and either standard (human insulin, HI) or insulin of the invention. The labelled glucose is converted into extractable lipids in a dose dependent way, resulting in full dose response curves. The result is expressed as relative potency (%) with 95% confidence limits of insulin of the invention compared to standard (HI).

Data are given in the table above.

Generally for the tested insulin derivatives according to the invention there is a very good accordance between the obtained lipogenesis data (in presence of 0.1% HSA) and the insulin receptor data obtained in presence of 1.5% HSA, thus confirming that the receptor binding is translated into receptor activation.

### Example 102

### Chemical Stability of Insulin Derivatives Formulated in Lipid Formulations

Chemical stability of insulin derivatives formulated in lipid formulations was assessed according to the protocol described here.

Preparation of Formulations:

Composition of the Formulation (SNEDDS Formulation):

Insulin to be tested (600 nmol/g)

15% Propylene glycol

30% Polysorbate 20

55% Diglycerol monocaprylate

The insulin to be tested (lyophilised from pH 7.5) is dissolved in propylene glycol in the dark for 16 hours. Diglycerol caprylate is added and the mixture is stirred. Polysorbate 20 is added and the mixture is stirred for 5 minutes. The mixture is gently agitated until it is homogeneous.

Each formulation is then aliquoted into 5 airtight cartridges and placed at various temperatures for a number of weeks. An example is given below.

Assays:

Sample Preparation for Purity and HMWP Analysis:

The SNEDDS formulations are allowed to reach room temperature. Each sample of SNEDDS formulation is diluted in 5% (v/v) ethanol: 25 μl SNEDDS formulation is transferred into an Eppendorf tube, and weight of the transferred SNEDDS formulation is noted, and then 975 μl 5% (v/v) ethanol is added. The mixture was thoroughly mixed using a vortexer.

The samples were analysed for purity and HMWP content using the methods described below.

Purity method:

Column: Waters Acquity CSH C18 column (2.1×150 mm, 1.7 μm)

Wavelength: 215 nm

Column temperature: 50° C.

Flow: 0.3 ml/min

Run-time: 60 min

Injection volume: 7 μl

Eluent A: 0.09M di-ammonium hydrogen phosphate pH 3.6, 10% (v/v) acetonitrile

Eluent B: 80% (v/v) acetonitrile

| Time (min) | (Flow ml/min) | % A | % B | Curve |
|---|---|---|---|---|
| 0 | 0.3 | 85 | 15 | — |
| 4 | 0.3 | 67 | 33 | 6 |
| 33 | 0.3 | 57 | 43 | 6 |
| 42 | 0.3 | 46 | 54 | 6 |
| 47 | 0.3 | 10 | 90 | 6 |
| 51 | 0.3 | 10 | 90 | 6 |
| 52 | 0.3 | 85 | 15 | 6 |
| 60 | 0.3 | 85 | 15 | 11 |
| 65 | 0.02 | 50 | 50 | 11 |

HMWP Method:

Column: Waters Acquity BEH200 SEC column, 4.6×150 mm

Wavelength: 215 nm

Flow: 0.2 ml/min

Run-time: 30 min

Column temperature: 40° C.

Injection volume: 10 μl

Eluent: 55% (v/v) acetonitrile, 0.05% TFA

Results:

In the table below is listed the increase in total impurities and HMWP, respectively, after 1 and 3 week incubation at 5° C. and 40° C.:

| | ΔImpurities (%) | | | | ΔHMWP (%) | | | |
|---|---|---|---|---|---|---|---|---|
| | 5° C. | | 40° C. | | 5° C. | | 40° C. | |
| | 1W | 3W | 1W | 3W | 1W | 3W | 1W | 3W |
| Formulation A | 3.29 | 2.92 | 16.26 | 27.55 | 0.18 | 0.31 | 6.22 | 12.67 |
| Formulation B | 1.51 | 1.68 | 3.19 | 8.80 | −0.03 | 0.03 | 0.31 | 0.93 |
| Formulation C | 1.84 | 3.30 | 8.73 | 21.30 | −0.06 | −0.06 | 0.07 | 0.40 |
| Formulation D | 2.59 | 0.83 | 14.54 | 24.50 | 0.08 | 0.01 | 5.18 | 10.48 |
| Formulation E | −0.07 | 2.27 | 7.52 | 22.03 | 0.08 | 0.24 | 0.23 | 0.77 |

Formulations A-E contains the following insulin derivatives:
A: Compound of example 1 and 34 without N-terminal protection: A10C, A14E, B3C, B25H, desB27, B29K (N^{ε}octadecanedioyl-gGlu), desB30 human insulin.
B: Compound of example 34: A1 (N^{α}Carbamoyl), A10C, A14E, B1 (N^{α}carbamoyl), B3C, B25H, desB27, B29K (N^{ε}octadecanedioyl-gGlu), desB30 human insulin.
C: Compound of example 1: A1 (N^{α}, N^{α}-Dimethyl), A10C, A14E, B1 (N^{α}, N^{α}-dimethyl), B3C, B25H, desB27, B29K (N^{ε}octadecanedioyl-gGlu), desB30 human insulin.
D: Compound of example 11 without N-terminal protection: A10C, A14E, B3C, B25H, desB27, B29K (N^{ε}octadecanedioyl-gGlu-2xOEG), desB30 human insulin.
E: Compound of example 11:A1 (N^{α}, N^{α}-Dimethyl), A10C, A14E, B1 (N^{α}, N^{α}-dimethyl), B3C, B25H, desB27, B29K (N^{ε}octadecanedioyl-gGlu-2xOEG), desB30 human insulin.

It is concluded that N-terminal protection protects the analogues of the invention form chemical degradation, both as measured for appearance (formation) of impurities and to a larger extent against appearance (formation) of HMWP (high molecular weight products) as compared with similar compounds without N-terminal protection.

In the following figures the data in the table above have been drawn as graphs for formulations A to E both with respect to formation of impurities and formation of HMWP products as a function of time (1 and 3 weeks) and as function of temperature (5° C. and 40° C.).

### Example 103

### Rat Pharmacokinecics, Intravenous Rat PK

Anaesthetized rats are dosed intravenously (i.v.) with insulin analogs at various doses and plasma concentrations of the employed compounds are measured using immunoassays or mass spectrometry at specified intervals for 4-6 or up to 48 hours or more post-dose. Pharmacokinetic parameters are subsequently calculated using WinNonLin Professional (Pharsight Inc., Mountain View, Calif., USA).

Non-fasted male Wistar rats (Taconic) weighing approximately 200 gram are used.

Body weight is measured and rats are subsequently anaesthetized with Hypnorm/Dormicum (each compound is separately diluted 1:1 in sterile water and then mixed; prepared freshly on the experimental day). Anaesthesia is initiated by 2 ml/kg Hypnorm/Dormicum mixture sc followed by two maintenance doses of 1 ml/kg sc at 30 min intervals and two maintenance doses of 1 ml/kg sc with 45 min intervals. If required in order to keep the rats lightly anaesthetised throughout a further dose(s) 1-2 ml/kg sc is supplied. Weighing and initial anaesthesia is performed in the rat holding room in order to avoid stressing the animals by moving them from one room to another.

### Example 104

### Rat Pharmacokinetics, Rat PK Following Intraintestinal Injection

Anaesthetized rats are dosed intraintestinally (into jejunum) with insulin analogs. Plasma concentrations of the employed compounds as well as changes in blood glucose are measured at specified intervals for 4 hours or more post-dosing. Pharmacokinetic parameters are subsequently calculated using WinNonLin Professional (Pharsight Inc., Mountain View, Calif., USA).

Male Sprague-Dawley rats (Taconic), weighing 250-300 g, fasted for ˜18 h are anesthetized using Hypnorm-Dormicum s.c. (0.079 mg/ml fentanyl citrate, 2.5 mg/ml fluanisone and 1.25 mg/ml midazolam) 2 ml/kg as a priming dose (to timepoint −60 min prior to test substance dosing), 1 ml/kg after 20 min followed by 1 ml/kg every 40 min.

The insulins to be tested in the intraintestinal injection model are formulated as formulated for the gavage model above.

The anesthetized rat is placed on a homeothermic blanket stabilized at 37° C. A 20 cm polyethylene catheter mounted a 1-ml syringe is filled with insulin formulation or vehicle. A 4-5 cm midline incision is made in the abdominal wall. The catheter is gently inserted into mid-jejunum ˜50 cm from the caecum by penetration of the intestinal wall. If intestinal content is present, the application site is moved ±10 cm. The catheter tip is placed approx. 2 cm inside the lumen of the intestinal segment and fixed without the use of ligatures. The intestines are carefully replaced in the abdominal cavity and the abdominal wall and skin are closed with autoclips in each layer. At time 0, the rats are dosed via the catheter, 0.4 ml/kg of test compound or vehicle.

Blood samples for the determination of whole blood glucose concentrations are collected in heparinised 10 μl capillary tubes by puncture of the capillary vessels in the tail tip. Blood glucose concentrations are measured after dilution in 500 μl analysis buffer by the glucose oxidase method using a Biosen autoanalyzer (EKF Diagnostic Gmbh, Germany). Mean blood glucose concentration courses (mean±SEM) are made for each compound.

Samples are collected for determination of the plasma insulin concentration. 100 μl blood samples are drawn into chilled tubes containing EDTA. The samples are kept on ice until centrifuged (7000 rpm, 4° C., 5 min), plasma is pipetted into Micronic tubes and then frozen at 20° C. until assay. Plasma concentrations of the insulin analogs are measured in a immunoassay which is considered appropriate or validated for the individual analog.

Blood samples are drawn at t=−10 (for blood glucose only), at t=−1 (just before dosing) and at specified intervals for 4 hours or more post-dosing.

### Example 105

### Potency of the Acylated Insulin Analogues of this Invention Relative to Human Insulin

Sprague Dawley male rats weighing 238-383 g on the experimental day are used for the clamp experiment. The rats have free access to feed under controlled ambient conditions and are fasted overnight (from 3 pm) prior to the clamp experiment.

Experimental Protocol:

The rats are acclimatized in the animal facilities for at least 1 week prior to the surgical procedure. Approximately 1 week prior to the clamp experiment, Tygon catheters are inserted under halothane anaesthesia into the jugular vein (for infusion) and the carotid artery (for blood sampling) and exteriorised and fixed on the back of the neck. The rats are given Streptocilin vet. (Boehringer Ingelheim; 0.15 ml/rat, i.m.) post-surgically and placed in an animal care unit (25° C.) during the recovery period. In order to obtain analgesia, Anorphin (0.06 mg/rat, s.c.) is administered during anaesthesia and Rimadyl (1.5 mg/kg, s.c.) is administered after full recovery from the anaesthesia (2-3 h) and again once daily for 2 days.

At 7 am on the experimental day overnight fasted (from 3 pm the previous day) rats are weighed and connected to the sampling syringes and infusion system (Harvard 22 Basic pumps, Harvard, and Perfectum Hypodermic glass syringe, Aldrich) and then placed into individual clamp cages where they rest for ca. 45 min before start of experiment. The rats are able to move freely on their usual bedding during the entire experiment and have free access to drinking water. After a 30 min basal period during which plasma glucose levels were measured at 10 min intervals, the N-terminally modified insulin to be tested and human insulin (one dose level per rat, n=6-7 per dose level) are infused (i.v.) at a constant rate for 300 min. Optionally a priming bolus infusion of the N-terminally modified insulin to be tested is administered in order to reach immediate steady state levels in plasma. The dose of the priming bolus infusion can be calculated based on clearance data obtained from i.v. bolus pharmacokinetics by a pharmacokinetician skilled in the art. Plasma glucose levels are measured at 10 min intervals throughout and infusion of 20% aqueous glucose is adjusted accordingly in order to maintain euglyceamia. Samples of re-suspended erythrocytes are pooled from each rat and returned in about ½ ml volumes via the carotid catheter.

On each experimental day, samples of the solutions of the individual N-terminally modified insulins to be tested and the human insulin solution are taken before and at the end of the clamp experiments and the concentrations of the peptides are confirmed by HPLC. Plasma concentrations of rat insulin and C-peptide as well as of the N-terminally modified insulin to be tested and human insulin are measured at relevant time points before and at the end of the studies. Rats are killed at the end of experiment using a pentobarbital overdose.

### Example 106

### Potency of the Acylated N-Terminally Modified Insulins of this Invention Relative to a Control N-Terminally Modified Insulin, Subcutaneous Administration to Rats

Male Sprague-Dawley rats (n=6 per group) receives a single dose subcutaneously of vehicle or insulin derivative (50 or 200 nmol/animal for derivatives with a medium duration of action or long duration of action, respectively). Blood (sublingual) is drawn and plasma collected at time points 0, 1, 2, 4, 8, 24 and 48 or 0, 2, 4, 8, 24, 48, 72, 96 hours after dosing, for derivatives with a medium duration of action or long duration of action, respectively). Plasma is assayed for glucose. The glucose lowering effect is calculated as the area under the curve of -delta plasma glucose as a function of time and compared to a control N-terminally modified insulin.

### Example 107

### Dog pharmacokinetics, Intravenous Dog PK

Male Beagle dogs (approximately 12 kg) receives a single dose intravenously of insulin insulin derivative (2 nmol/kg). Blood is drawn and plasma collected at time points −0.17, 0, 0.083, 0.25, 0.5, 0.75, 1, 1.25, 1.5, 2, 2.5, 3, 3.5, 4, 5, 8, 10, 12, 16, 24, 32, 48, 72, 96, 120, 144 and 168 hours after dosing. Plasma samples are analyzed by either sandwich immunoassay or LCMS. Plasma concentration-time profiles are analysed by non-compartmental pharmacokinetics analysis using WinNonlin Professional 5.2 (Pharsight Inc., Mountain View, Calif., USA).

### Example 108

### Dog pharmacokinetics, Oral Dosing

Male Beagle dogs (approximately 12 kg) receives a single dose orally of insulin derivative (120 nmol/kg) formulated in an enteric coated capsule, size 00. Blood is drawn and plasma collected at time points 0, 15, 30, 45, 60, 75, 90, 105, 120, 135, 150, 165, 180, 210, 240, 270, 300, 360, 480, 600, 720, 1440 minutes (24 h), 30 h, 48 h and 72 h after dosing. Plasma samples are analyzed by either sandwich immunoassay or LCMS. Plasma concentration-time profiles are analysed by non-compartmental pharmacokinetics analysis using WinNonlin Professional 5.2 (Phar-sight Inc., Mountain View, Calif., USA).

### Example 109

### Melting Temperature Determinations

### Differential Scanning Calorimetry (DSC).

Data collection was performed using a VP-DSC differential scanning microcalorimeter (MicroCal, LLC, Northampton, Mass.). All protein scans (˜200 μM N-terminally modified insulins) were performed with 2 mM phosphate buffer in the reference cell from 15° C. to 120° C. at a scan rate of 1° C./min and an excess pressure of 0.21 MPa. All samples and references were degassed immediately before use. A buffer-buffer reference scan was subtracted from each sample scan prior to concentration normalization.

DSC data are shown in FIGS. 3 and 4

### Example 110

### Measurement of Tendencies of Fibrillation

### General Procedure for Thioflavin T (ThT) Fibrillation Assay:

### Principle

Low physical stability of a peptide may lead to amyloid fibril formation, such is observed as well-ordered, thread-like macromolecular structures in the sample eventually resulting in gel formation. This has traditionally been measured by visual inspection of the sample. However, that kind of measurement is very subjective and depending on the observer. Therefore the application of a small molecule indicator probe is much preferred. Thioflavin T (ThT) is such a probe and has a distinct fluorescence signature when binding to fibrils (Naiki et al. Anal. Biochem. 177, 244-249, 1989; Le-Vine, Methods Enzymol. 309, 274-284, 1999). The time course for fibril formation can be described by a sigmoidal curve with the following expression (Nielsen at al. Biochemistry 40, 6036-6046, 2001):

$$F = {f}_{i} + {m}_{i} t + \frac{{f}_{f} + {m}_{f} t}{1 + {e}^{- [ ( t - {t}_{0} ) / τ ]}} \qquad Chem . VIII$$

Here, F is the ThT fluorescence at the time t. The constant t₀ is the time needed to reach 50% of maximum fluorescence. The two important parameters describing fibril formation are the lag-time calculated by t₀-2□ and the apparent rate constant kₐₚₚ=1/\tilde{□}.

Formation of a partially folded intermediate of the peptide is suggested as a general initiating mechanism for fibrillation. Few of those intermediates nucleate to form a template onto which further intermediates may assemble and the fibrillation proceeds. The lag-time corresponds to the interval in which the critical mass of nucleus is built up and the apparent rate constant is the rate with which the fibril itself is formed.

### Sample Preparation

Samples were prepared freshly before each assay. Each analog was dissolved in 2 mM sodium phosphate, pH=7.4. Thioflavin T was added to the samples from a stock solution in water to a final concentration of 1 μM. Sample aliquots of 200 μl were placed in a 96 well microtiter plate (Packard Optiplate™-96, white polystyrene). Usually four replica of each sample (corresponding to one test condition) were placed in one column of wells. The plate was sealed with Scotch Pad (Qiagen).

### Incubation and Fluorescence Measurements

Incubation at given temperature, shaking and measurements of the ThT fluorescence emission were done in either a Fluoroskan Ascent FL or Varioskan fluorescence plate reader (thermo Labsystems). The temperature was adjusted to 37° C. The orbital shaking was adjusted to 960 rpm with an amplitude of 1 mm in all the presented data. Fluorescence measurements were done using excitation through a 444 nm filter and measurement of emission through a 485 nm filter.

Each run was initiated by incubating the plate at the assay temperature for 10 min. The plate was measured every 20 minutes for typically 45 hours. Between each measurement, the plate was shaken and heated as described above.

### Data Handling

The measurement data points were saved in Microsoft Excel format for further processing and curve drawing and fitting was performed using Graph Pad Prism. The background emission from ThT in the absence of fibrils was negligible. The data points are typically a mean of four samples. Only data obtained in the same experiment (i.e. samples on the same plate) are presented in the same graph ensuring a relative measure of fibrillation between the individual samples of one assay rather then comparison between different assays. The data set may be fitted to Eq. (1). However since the full sigmoidal curves are not usually achieved during the measurement time, the degree of fibrillation is expressed as ThT fluorescence at various time points calculated as the mean of the four samples and shown with the standard deviation

Results obtained in a previous experiment for human insulin:

| | 0 h | 2 h | 20 h | 45 h |
|---|---|---|---|---|
| Human insulin | 28 ± 1 | 1567 ± 46 | 1780 ± 40 | 1732 ± 48 |

Results obtained for insulin derivatives of the prior art. ThT assays were conducted as two times 20 h assays. The stated time points are measured from initiation of the first 20 h part.

| | 0 h | 5 h | 10 h | 30 h | 40 h |
|---|---|---|---|---|---|
| A14E, B25H, desB27, | 34 ± 0.6 | 15 ± 1.5 | 15 ± 1.7 | 208 ± 317 | 647 ± 432 |
| B29K(N^{ε}Octadecanedioyl-gGlu-2xOEG), | | | | | |
| desB30 human insulin | | | | | |
| (WO 09115469, Example 57) | | | | | |
| A14E, B25H, desB27, | 22 ± 0.9 | 273 ± 446 | 1326 ± 195 | 1477 ± 217 | 1483 ± 244 |
| B29K(N^{ε}Octadecanedioyl-gGlu), desB30 | | | | | |
| human insulin | | | | | |
| (WO 09115469, Example 151) | | | | | |
| A1G(N^{α},N^{α}-dimethyl), B1F(N^{α},N^{α}- | 23 ± 0.2 | 1762 ± 92 | 1764 ± 108 | 1839 ± 96 | 1829 ± 99 |
| dimethyl), B29K(N^{ε}hexadecanedioyl- | | | | | |
| gGlu), desB30 human insulin | | | | | |
| (WO 08145721, Example 3) | | | | | |
| A14E, B25H, B29K(N^{ε}Eicosanedioyl- | 37 ± 0.2 | 24 ± 1.8 | 197 ± 199 | 1117 ± 92 | 1265 ± 82 |
| gGlu-2xOEG), desB30 human insulin | | | | | |
| (WO 09115469, Example 151) | | | | | |

Results obtained for insulin derivatives of the invention in the same experiment as for the prior art insulin derivatives

| Example | | | | | |
|---|---|---|---|---|---|
| No. | ThT 0 h | ThT 5 h | ThT 10 h | ThT 30 h | ThT 40 h |
| 34 | 20 ± 0.1 | 13 ± 0.7 | 13 ± 0.8 | 13 ± 0.4 | 13 ± 0.3 |
| 1 | 25 ± 1.4 | 14 ± 0.5 | 46 ± 38 | 73 ± 6 | 73 ± 6 |
| 11 | 31 ± 0.4 | 23 ± 0.4 | 23 ± 0.6 | 21 ± 0.2 | 21 ± 0.3 |
| 2 | 35 ± 0.9 | 22 ± 0.4 | 22 ± 0.3 | 20 ± 0.3 | 19 ± 0.3 |

It is evident that none of the insulins according to the invention that were tested towards fibrillation in the ThT assay showed any signs of fibrillation as seen by (absence of) increased fluorescence as a function of time. This is very unusual and this understates the utility of the derivatives of the invention.

### Example 111

### Chemical Stability of the Insulin Derivatives of the Invention

Chemical stability of an insulin derivative is assessed after incubating insulin derivative in 2 mM phosphate, pH=7.5 at 37° C. for up to 8 weeks. Formation of high molecular weight products (HMWP) is determined by SEC HPLC analysis after 0, 2, 4 and optionally 8 weeks. The results of the SEC method are given as a difference between HMWP formation at 37° C. and 5° C. start sample as a percentage of total absorbance at 215 nm. Chemical degradation products are determined by RP HPLC analysis after 0, 2, 4 and optionally 8 weeks. The results of the RP method are given as a difference between chemical degradation observed at 37° C. and 5° C. start sample as a percentage of total absorbance at 215 nm.

### SEC-HPLC Method:

Solvent: 500 mM NaCl, 10 mM NaH₂PO₄, 5 mM H₃PO₄, 50% (v/v) 2-propanol
Flow: 0.5 ml/min
Run time: 30 min
UV Detection: 215 nm
Column: Insulin HMWP column from Waters 7.8×300 mm
Temperature: 50° C.

### RP-HPLC Method:

Solvent A: 0.09M phosphate buffer pH 3.6 (di-ammoniumhydrogenphosphate), 10% MeCN (v/v)
Solvent B: 80% MeCN (v/v %)
Flow: 0.3 ml/min
Runtime: 33 min
UV Detection: 215 nm
Column: Waters Acquity BEH130 C18 Column 1.7 μm, 150×2.1 mm
Temperature: 50° C.
Gradient:

| Time, min | Flow, ml/min | % A | % B |
|---|---|---|---|
| 0 | 0.3 | 95 | 5 |
| 2 | 0.3 | 95 | 5 |
| 25 | 0.3 | 45 | 55 |
| 27 | 0.3 | 20 | 80 |
| 28 | 0.3 | 20 | 80 |
| 29 | 0.3 | 95 | 5 |
| 33 | 0.3 | 95 | 5 |

| | Chemical | HMWP |
|---|---|---|
| | degradation (%) | Formation (%) |
| | (4 weeks 37° C.- | (4 weeks 37° C.- |
| Example No. | 0 weeks 5° C.) | 0 weeks 5° C.) |
| 1 without A10-B4 | 11.7 | 1.0 |
| disulfide bridge | | |
| (prior art) | | |
| 18 | 4.2 | 5.1 |
| 19 | 11.7 | 34.6 |
| 1 | 6.8 | 0.9 |
| 2 | 2.5 | 0.0 |
| 3 | 3.4 | 0.7 |
| 11 | 3.2 | −0.2 |
| 12 | 7.3 | −0.1 |
| 13 | 9.8 | 0.5 |
| 14 | 9.7 | 0.0 |
| 4 | 8.7 | −0.4 |
| 9 | 5.7 | 0.7 |
| 15 | 10.4 | 1.2 |
| 16 | 58.0 | 42.7 |
| 5 | 12.5 | 0.4 |
| 17 | 13.3 | 0.2 |
| 7 | 0.6 | 0.4 |
| 8 | 66.4 | 54.5 |
| 24 | 2.0 | 0.4 |
| 25 | 1.9 | 0.8 |

It is concluded that s with a disulfide to B3 are in general more stable than s with disulfide to B2 or B1 are less stable.

### Example 112

### X-Ray Structure Determination

An example of crystallization conditions is given bellow, however, the exact conditions could be different for different derivatives and the optimal conditions are found by screening many different conditions. Crystals are obtained by the sitting drop vapor diffusion method from, for example a reservoir solution containing 0.8 M K/NaTartrate, 0.1 M Tris pH 8.5, 0.5% PEG MME 5000. Data are collected with a rotating anode (Rigaku, MicroMax-007HF) equipped with a MarCCD detector and processed by XDS (J Appl Crystallogr 26: 795-800). The structure is solved by molecular replacement using Molrep (J Appl Crystallogr 30: 1022-1025) with an in house structure as search model. Data refinement and model building is made using the programs Refmac (Acta Crystallogr D 53: 240-255) and Coot (Acta Crystallogr D 60: 2126-2132).

### Example 113

### Guanidinium Hydrochloride Denaturation

Guanidinium hydrochloride denaturation of selected N-terminally modified insulins containing extra disulfide bonds can be followed by circular dichroism (CD) spectroscopy in order to determine free energy of unfolding. Upon protein denaturation, negative CD in the far UV range (240-218-nm) gradually diminishes, consistent with the loss of ordered secondary structure that accompanies protein unfolding. The far-UV CD spectrum of human insulin is sensitive to both protein unfolding and self-association (Holladay et al., 1977 Biochim. Biophys. Acta 494, 245-254; Melberg & Johnson, 1990, Biochim. Biophys. Acta 494, 245-254). In order to separate these phenomena at pH 8, GuHCl titrations is carried out at different protein concentrations, e.g. 3, 37, and 250 μM. At these concentrations, insulin analogues exists mainly as monomers, dimers and mixture of dimers and higher aggregates. The insulin CD spectrum in the near UV range (330-250-nm) reflects the environment of the tyrosine chromophore with contributions from the disulfide linkages (Morris et al., 1968, Biochim. Biophys. Acta. 160, 145-155; Wood et al., 1975, Biochim. Biophys. Acta. 160, 145-155; Strickland & Mercola, 1976, Biochemistry 15, 3875-3884). Plots of changes in molar ellipticities at both near UV and far UV regions as a function of denaturant concentration is be generated. Free energy of unfolding was previously calculated from such insulin denaturation curves fitted with two state model (Kaarsholm, N. C. et al 1993 Biochemistry, 32, 10773-8).

Protein concentrations is determined by UV absorbance and/or RP-HPLC and/or SEC-HPLC. Denaturation samples are prepared by combining different ratios of protein and GuHCl stock solutions with 10 mM Tris/C104-buffer, pH 80. Protein stock solutions are typically 1.5 mM in 10 mM Tris/C104-, pH 8.0. GuHCl stock solutions are 8.25 M (determined by refractometry) in 10 mM Tris/C104-, pH 8.0. All CD spectra are recorded at 25° C. Far-UV CD denaturation samples are scanned from 250 to 218 nm. Typical cell path length and protein concentrations are 0.2 cm and 37 pM, respectively. Near-UV CD denaturation samples are scanned from 330 to 250 nm using I-cm path length and typically 75 pM protein. All spectra are smoothed by a Fourier transform algorithm before subtraction of the appropriate solvent blanks. In the far-UV range, Ae is based on the molar concentration of peptide bond, while in the near-UV Δε is normalized to the molar concentration of insulin monomer.

GuHCl denaturation curves are analyzed by assuming that the folding/unfolding transition is two-state, in which case equilibrium constants can be obtained at each denaturant concentration using K=(Δε_{N}−Δε)(Δε−Δε_{U}), where Δε is the observed value of the CD and Δε_{N} and Δε_{U} represent the CD values for native and unfolded forms, respectively, at the given GuHCl concentration (Pace, C. N. (1975) CRC Crit. Rev. Biochem. 3, 1-43). Values for Δε_{N} and Δε_{U} at GuHCl concentrations in the transition region were obtained by linear extrapolation of the pre- and posttransition base lines into the transition region, ie., Δε_{N}=Δε^{o}_{N}+m_{N}[GuHCl] and Δε_{U}=Δε^{o}_{U}+m_{U}[GuHCl], where Δε^{o}_{N} and Δε^{o}_{U} are intercepts and m_{N} and m_{U} are slopes of the pre- and posttransition base lines, respectively. The free energy of unfolding at a given denaturant concentration in the transition zone is given by ΔG=−RT In K. We assume a linear dependence of ΔG on denaturant concentration: ΔG=ΔG_{H2O}−m[GuHCl], where ΔG_{H2O} is the value of ΔG in the absence of denaturant and m is a measure of the dependence of ΔG on denaturant concentration. Hence, ΔG values derived from K in the transition zone may be extrapolated back to 0 M denaturant to give ΔG_{H2O}. The relationship between Δε and [GuHCl] for the complete unfolding curve is shown in eq 1 (Santoro, M. M., & Bolen, D. W. (1988) Biochemistry 27, 8063-8068):

Δε={(Δε^{o}_{N}+m_{N}[GuHCl])+(Δε^{o}_{U}+m_{U}[GuHCl])exp[−(ΔG_{H2O}−m[GuHCl])RT)}/{(I+exp[−(ΔG_{H2O}−m[GuHCl])/RT]}

With Δε as the response and [GuHCl] as the independent variable, this equation is subjected to nonlinear least-squares analysis using, for example the NLIN procedure of PC SAS (SAS Inc., Cary, N.C.). Six parameters then describe the denaturation curve: Δε^{o}_{N}, Δε^{o}_{U}, m_{N}, m_{U}, m, and ΔG_{H2O}. In addition, the GuHCl concentration at the midpoint of the denaturation curve, C_{mid}, is given by ΔG_{H2O}/m. The difference in the free energy of unfolding between human and mutant insulins may then be calculated from ΔΔG_{H2O}=ΔG_{H2O}(mutant)−ΔG_{H2O}(wild type).

### Example 114

### Accurate Intact Mass Determination

LC-MS instrumentation consists of Acquity UPLC system (Waters, Milford, Mass.) and Synapt G2 mass spectrometer (Waters, Milford, Mass.). Insulin derivatives are applied to a C18 reversed-phase HPLC column and analyzed using a linear gradient of acetonitrile in 0.05% trifluoroacetic acid. The flow from HPLC is applied directly to the electrospray interface of the Synapt G2 operating in the positive MS only mode with 2500 V capillary potential, 110° C. source temperature, 250° C. desolvation temperature and cone gass flow (N₂) of 50 L/h. MS spectra from m/z=100 to m/z=3000 are acquired twice per second. Instrument is calibrated by a standard mixture of NaI prior to analyses and lock spray of leucine enkephalin is applied during LC-MS analyses. Intact insulin masses are reconstructed by BioPharmaLynx 1.2 (Waters, Milford, Mass.) using MaxEnt3 algorithm. Orbitrap XL mass spectrometer (Thermo Fisher) can be used instead of Synapt G2. Orbitrap instrument is operated in the positive MS mode with source voltage of 4 kV, source current of 100 μA, sheath gas flow of 40, auxilliary gas flow of 10, sweep gas flow of 5, capillary voltage of 20 V. All MS parameters are adjusted during tuning of the instruments for optimal performance and may deviate slightly from those given above. Mass accuracy obtained by this method is better than 10 ppm.

Column: Acquity BEH C18 1×150 mm, 1.7 μm (Waters)

Flow: 0.1 ml/min

Buffer A: 0.02% (v/v) or 0.05% (v/v) TFA

Buffer B: 0.02% (v/v) or 0.04% (v/v) TFA in acetonitrile

Gradient: 5% B for 2 min; 5% B to 50% B in 12 min, 50% B to 90% B in 1 min

UV Detection: 215 nm

1. An N-terminally modified insulin consisting of a peptide part, N-terminal modification groups and an albumin binding moiety, wherein the peptide part has at least one disulphide bond which is not present in human insulin.

2. An N-terminally modified insulin consisting of a peptide part, N-terminal modification groups and an albumin binding moiety, wherein the peptide part has two or more cysteine substitutions and the three disulfide bonds of human insulin are retained.

3. An N-terminally modified insulin according to claim 1, wherein the sites of cysteine substitutions are chosen in such a way that the introduced cysteine residues are placed in the three dimensional structure of the folded N-terminally modified insulin to allow for the formation of one or more additional disulfide bonds not present in human insulin, and wherein the N-terminally modified insulin has at least 5% of the insulin receptor affinity of an insulin peptide having the same peptide part, the same N-terminal modification groups and the same albumin binding moiety but without any disulphide bonds which are not present in human insulin.

4. An N-terminally modified insulin according to claim 1, wherein the modification groups are one or two organic substituents which are each having a molecular weight (MW) below 200 g per mol conjugated to the N-terminals of the parent insulin.

5. An N-terminally modified insulin according to claim 1, wherein modification groups are designated Y′ and Z in Chem. V (illustrated for the A-chain N-terminal): 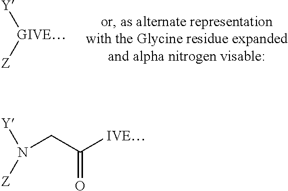
and wherein Y′ and Z are attached to the N-terminal amino acids of the insulin peptide.

6. An N-terminally modified insulin according to claim 5, wherein Y′ and Z are different and
Y′ is R—C(═X)—,
Z is H,
R is H, NH₂, straight chain or branched C1-C4 alkyl, straight chain or branched C2-C4 alkyl substituted with dimethylamino, diethylamino, dipropylamino, dimethylammonium, diethylammonium, or dipropylammonium, CO₂H, or —OCH₂CO₂H, C5-C6 cycloalkyl, substituted C5-C6 cycloalkyl, 5- or 6 membered saturated heterocyclyl, substituted 5- or 6 membered saturated heterocyclyl, and
X is O or S.

7. An N-terminally modified insulin according to claim 5, wherein Y′ and Z are different and
Y′ is R—C(═X)—,
Z is H,
R is H, NH₂, straight chain or branched C1-C4 alkyl, C1-C4 alkyl substituted with CO₂H, or —OCH₂CO₂H, and
X is O.

8. An N-terminally modified insulin according to claim 5, wherein Y′ and Z are the same and methyl.

9. An N-terminally modified insulin according to claim 1, wherein the N-terminal modification group is selected from the group consisting of: N,N-dimethyl, N,N-diethyl, carbamoyl, formyl, acetyl, propionyl, butyryl, glutaryl, and diglycolyl.

10. An N-terminally modified insulin according to claim 2, wherein the positions for cysteine substitution are A10C and B3C.

11. An N-terminally modified insulin according to claim 2, wherein the peptide part in addition to two or more cysteine substitutions comprises one or more substituted amino acids selected from the group consisting of: A14E, B16H, B25H, desB27, and desB30.

12. An N-terminally modified insulin according to claim 1, wherein the peptide part is selected from the group consisting of: A10C, A14E, B3C, B16H, B25H, desB30 human insulin; A10C, A14E, B3C, B25H, desB27, desB30 human insulin; A10C, A14E, B3C, B25H, desB30 human insulin; A10C, A14E, B3C, desB27, desB30 human insulin; A10C, A14E, B3C, B16H, B25H, desB27, desB30 human insulin; and A10C, A14E, B16H, desB27, desB30 human insulin.

13. An N-terminally modified insulin according to claim 1, wherein the albumin binding moiety has the general formula Acy-AA1ₙ-AA2ₘ-AA3ₚ- (Chem. IV),
wherein
n is 0 or an integer in the range from 1 to 3;
m is 0 or an integer in the range from 1 to 10;
p is 0 or an integer in the range from 1 to 10;
Acy is a fatty acid or a fatty diacid comprising from about 14 to about 20 carbon atoms;
AA1 is a neutral linear or cyclic amino acid residue;
AA2 is an acidic amino acid residue;
AA3 is a neutral, alkyleneglycol-containing amino acid residue;
the order by which AA1, AA2 and AA3 appears in the formula can be interchanged independently; AA2 can occur several times along the formula (e.g., Acy-AA2-AA3₂-AA2-); AA2 can occur independently (=being different) several times along the formula (e.g., Acy-AA2-AA3₂-AA2-); the connections between Acy, AA1, AA2 and/or AA3 are amide (peptide) bonds which, formally, can be obtained by removal of a hydrogen atom or a hydroxyl group (water) from each of Acy, AA1, AA2 and AA3; and attachment to the peptide part can be from the C-terminal end of a AA1, AA2, or AA3 residue in the acyl moiety of the Chem. IV or from one of the side chain(s) of an AA2 residue present in the moiety of Chem. IV.

14. An N-terminally modified insulin according to claim 1, which is selected from the group consisting of:
A1(N^{α},N^{α}-Dimethyl), A10C, A14E, B3C, B25H, desB27, B29K(N^{ε}octadecanedioyl-gGlu-2×OEG), desB30 human insulin
A1(N^{α},N^{α}-Dimethyl), A10C, A14E, B1(N^{α},N^{α}-dimethyl), B3C, B25H, B29K(N^{ε}eicosanedioyl-gGlu-2×OEG), desB30 human insulin.

15. A pharmaceutical composition comprising an N-terminally modified insulin according to claim 1.
